(19) United States
(12) Patent Application Publication
(10) Pub. No.: US 2007/0225245 A1
(43) Pub. Date: Sep. 27, 2007

(54) Viral vector driven mutant bacterial cytosine deaminase gene and uses thereof

(76) Inventors: Donald J. Buchsbaum, Alabaster, AL (US); G. Yancey Gillespie, Birmingham, AL (US); James M. Markert, Birmingham, AL (US); Sergey A. Kaliberov, Birmingham, AL (US)

Correspondence Address: Benjamin Aaron Adler;ADLER & ASSOCIATES, 8011 Candle Lane, Houston, TX, 77071, US

(21) Appl. No.: 11/796,574

(22) Filed: Apr. 27, 2007

Related U.S. Application Data
(63) Continuation-in-part of application No. 10/795,551, filed on Mar. 8, 2004.
Application No. 10/795,551 is a division of application No. 10/304,436, filed on Nov. 26, 2002, now Pat. No. 6,703,375.
Application No. 10/304,436 is a continuation-in-part of application No. 09/706,190, filed on Nov. 3, 2000, now Pat. No. 6,552,005.
Application No. 09/706,190 is a continuation-in-part of application No. 09/408,055, filed on Sep. 29, 1999, now Pat. No. 6,599,909.
(60) Provisional application No. 60/102,391, filed on Sep. 29, 1998.

(51) Int. Cl. A61K 31/70; C12N 15/869

(52) U.S. Cl. 514/44; 435/320.1

(57) ABSTRACT

The instant invention has developed viral vectors encoding a mutant bacterial cytosine deaminase (bCD) gene, which have a higher affinity for cytosine than wild type bCD (bCDwt). The purpose of the present invention was to evaluate cytotoxicity in vitro and therapeutic efficacy in vivo of these vectors in combination with the prodrug 5-FC and ionizing radiation against human glioma. The present study demonstrates that infection with the viral vector expressing the mutant cytosine deaminase gene resulted in increased 5-FC-mediated cell killing, compared with vectors expressing the wild-type gene. Furthermore, a significant increase in cytotoxicity following infection with viral vector expressing the mutant cytosine deaminase gene and radiation treatment of glioma cells in vitro was demonstrated as compared to infection with viral vector expressing the wild-type gene. Animal studies showed significant inhibition of subcutaneous or intracranial tumor growth of D54MG glioma xenografts by the combination of AdbCD-D314A/5-FC with ionizing radiation as compared with either agent alone, and with AdbCDwt/5-FC plus radiation. These data indicate that combined treatment with this mutant enzyme/prodrug therapy and radiotherapy provides a promising approach for cancer therapy.

### CROSS-REFERENCE TO RELATED APPLICATION

This application is a continuation-in-part of U.S. Ser. No. 10/795,551, filed Mar. 8, 2004, which is a divisional of U.S. Ser. No. 10/304,436, filed Nov. 26, 2002 and issued as U.S. Pat. No. 6,703,375 on Mar. 9, 2004, which is a divisional of U.S. Ser. No. 09/706,190, filed Nov. 3, 2000 and issued as U.S. Pat. No. 6,552,005 on Apr. 22, 2003, which is a continuation-in-part of U.S. Ser. No. 09/408,055, filed Sep. 29, 1999 and issued as U.S. Pat. No. 6,599,909 on Jul. 29, 2003, which claims benefit of priority of provisional U.S. Ser. No. 60/102,391, filed Sep. 29, 1998, now abandoned.

### FEDERAL FUNDING LEGEND

This invention was created in part using funds from the federal government through National Institutes of Health grant P50-CA097247. The U.S. government, therefore, has rights in this invention.

### BACKGROUND OF THE INVENTION

1. Field of the Invention

The present invention relates generally to the fields of molecular biology, radiation oncology and cancer therapy. More specifically, the present invention relates to the finding that viral-directed enzyme/prodrug therapy in combination with radiation therapy enhances therapeutic effects against glioma.

2. Description of the Related Art

Malignant brain tumors pose a challenge to develop safe and effective therapies that can be integrated into the traditional therapeutic tripod of surgery, radiotherapy and chemotherapy. Their unusual resistance to radiation and chemotherapy, their highly invasive nature and a remarkable heterogeneity that reflects the genomic instability of these tumor cells contribute substantially to the fact that patient median survival has not changed appreciably despite aggressive therapeutic approaches. Glioblastoma multiforme is a most common and highly lethal primary neoplasm. Treatment generally consists of surgery and radiation therapy in combination with temozolomide. Despite advances in the treatment of malignant glioma, the prognosis remains poor. Thus, the development of more effective alternative treatments will be critical to improve the survival of patients with these tumors. Among these approaches, molecular chemotherapy or gene-directed enzyme-prodrug therapy (GDEPT) has received considerable attention [1].

The key element of a gene-directed enzyme-prodrug therapy is a gene that encodes an enzyme, which converts a prodrug to an active cytotoxic drug. Importantly, prodrug-activating enzymes are normally absent or poorly expressed in mammalian cells. This means tumor-targeting of gene therapy, using specific delivery vehicles, restricts enzyme expression to the transduced tumor cells and adjacent surrounding tumor cells through diffusion of the drug metabolite to generate a bystander effect. One of the most widely used suicide gene/prodrug systems for cancer utilizes cytosine deaminase (CD; EC 3.5.4.1) in combination with the antifungal agent 5-fluorocytosine (5-FC) that has been investigated intensely during the last decade [2]. Cytosine deaminase is a bacterial (b) or yeast (y) enzyme that can convert 5-FC into the chemotherapy agent 5-fluorouracil (5-FU), which is further processed by cellular enzymes into either 5-fluorouracil triphosphate (5-FUTP) or 5-fluoro-2′-deoxyuridine 5′-monophosphate (5-FdUMP). 5-FUTP is incorporated into RNA and interferes with RNA processing, while 5-FdUMP irreversibly inhibits thymidylate synthase and hence DNA synthesis. Importantly, 5-FU is able to diffuse across the cell membrane into adjacent cells without passing through gap junctions, resulting in a more powerful bystander effect [3]. Moreover, 5-FU is a strong radiosensitizer [4]. In the central nervous system, the vast majority of non-malignant cells are non-replicating and terminally differentiated, suggesting that gene therapy for glioma effecting termination of DNA synthesis would be tumor cell-specific. Adenoviral-mediated CD gene therapy has been studied for glioma treatment in vitro and in animal models [5-9].

A major problem associated with this suicide gene-directed enzyme-prodrug therapy approach is the low affinity displayed by the CD gene product toward 5-FC in comparison with cytosine. Thus, high doses of this prodrug must be administered in order to achieve cell killing. The plasma levels of 5-FC required to obtain a significant amount of active metabolites may lead to adverse effects. This is observed with 5-FC, whereas deamination by CD of bacterial intestinal microflora into 5-FU is responsible for side effects[10]. Fortunately, recent studies have demonstrated that substitution of an alanine (A) for the aspartic acid (D) at position 314 of bCD increased relative specificity of the mutant bCD-D314A enzyme to 5-FC in comparison with wild-type bCD (bCDwt) and may be a superior suicide gene [11,12].

The prior art is deficient in the lack of effective means of treating of human cancers by chemotherapy combined with radiation therapy to produce enhanced therapeutic effects against cancer and reduced normal tissue toxicity. Specifically, the prior art is deficient in the knowledge of the therapeutic efficacy of the mutant bCD/5-FC therapy alone or in combination with radiation therapy. The present invention fulfills this long-standing need and desire in the art.

### SUMMARY OF THE INVENTION

The present invention is directed to a method of infecting established tumors of the central nervous system with a virus encoding the mutant cytosine deaminase gene, administration of systemic 5-FC, and radiation therapy, (e.g., external beam or brachytherapy) of the tumor. The adenovirus as well as an aneurovirulent Herpes Simplex virus have been investigated as vectors for effective gene delivery by the present invention. The mutant cytosine deaminase has a decreased efficiency for the endogenous cytosine, which can compete with the prodrug for the active enzyme site, in combination with an increase for 5-FC that results in a greater fold substrate preference for 5-FC in comparison to the wild-type cytosine deaminase (CDwt). This method results in tumor regression and prolonged tumor growth inhibition compared to control treatments with molecular chemotherapy or radiation therapy alone. The present invention investigated replication deficient as well as replication competent adenoviruses and Herpes Simplex viruses as vectors. A main factor currently limiting the clinical potential of gene therapy is the poor level of in situ tumor cell transduction by existing gene transfer vectors. Methods to increase solid tumor transduction in situ may augment therapeutic gene expression and response to therapy. Gene delivery in the present invention was improved via vector binding to molecules expressed on tumor cells. The viral vectors encoding the mutant cytosine deaminase gene have been modified to express the RGD peptide in the fiber knob.

The present invention is directed to a recombinant adenovirus vector consisting of a gene encoding mutant cytosine deaminase operatively linked to a functional promoter; where the vector, when transfected in a host, expresses cytosine deaminase in a biologically active form. The present invention is also directed to a mutant Herpes Simplex Virus 1 vector consisting of a gene encoding cytosine deaminase; and a gene encoding uracil phosphoribosyl transferase; operatively linked to a functional promoter; where the vector when transfected to a host, expresses both the cytosine deaminase and uracil phosphoribosyl transferase in a biologically active form. The present invention is further directed to a method of causing selective growth inhibition of malignant tumor in a mammal consisting of introducing the genetically engineered vector of either of the compositions described supra in the mammal; where the product of the vector is expressed in the malignant tumor and administering 5-fluorocytosine, in the mammal. The present invention is also directed to a method of enhancing radiosensitization in a mammal in need thereof consisting of administering to the mammal a genetically engineered viral vector of the compositions described herein; administering 5-fluorocytosine to the mammal; and treating the individual with radiation therapy. Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows Ad-mediated suicide gene therapy increased radiation-induced glioma cell death.

Clonogenic survival assay of D54MG glioma cells. Twenty-four hours after infection with 50 MOI of AdbCDwt or AdbCD-D314A, 5-FC was added at 4 μg/ml and the next day cells were either mock-irradiated or irradiated at 2 Gy. Cells were fixed and colonies were counted at 21 days after treatment. Data are presented as percentage of colonies in comparison with mock-irradiated control. Presented are mean values±standard deviations of three independent experiments, each performed in six replicates. *p=0.003 for AdbCD-D314A in comparison with AdbCDwt plus 5-FC treated cells; **p=0.017 for AdbCD-D314A plus 5-FC in combination with radiation treatment in comparison with AdbCD-D314A plus 5-FC alone.

FIG. 2 shows the CD conversion activity in D54MG xenografts injected with AdbCD-D314A in combination with radiation. The CD enzyme activity was determined by measuring the conversion of ³H-5-FC to ³H-5-FU in lysates of D54MG glioma xenografts after intratumoral (i.t). infection with 1.5×10⁸ TCID₅₀ AdbCD-D314A on Day 0, and irradiated with 2 Gy using a ⁶⁰Co gamma irradiator one day before (2 Gy+AdbCD-D314A) or one day after Ad injection (AdbCD-D314A+2 Gy). Data points represent the mean CD conversion activity±standard deviations in each group of 6 animals. * p=0.018 for AdbCD-D314A alone compared to AdbCD-D314A+2 Gy treated group.

FIG. 3 shows growth of D54MG xenografts treated with AdbCD-D314A or AdbCDwt alone and in combination with ionizing radiation. Treatment was started at the time of established tumor growth (Day 0 equal to 14 days after tumor cell injection). Animals were infected i.t. with 1×10⁸ TCID₅₀ AdbCDwt or AdbCD-D314A on Days 0, 7, and 14, and then irradiated with 2 Gy on Days 4, 7 and 10. 5-FC was injected i.p. at 500 mg/kg on Days 0 to 4, 7 to 11, and 14 to 18. Data points represent the mean change in tumor surface area relative to Day 0 for each group of animals.

FIG. 4 shows growth of D54MG xenografts treated with AdbCD-D314A alone and in combination with ionizing radiation. Treatment was started at the time of established tumor growth (Day 0 equal to 17 days after tumor cell injection). Animals were infected i.t. with 1×10⁸ TCID₅₀ AdbCD-D314A on Days 0, 7, and 14, and irradiated with 5 Gy on Days 4, 7 and 10. 5-FC was injected i.p. at 500 mg/kg on Days 0 to 4, 7 to 11, and 14 to 18. Data points represent the mean change in tumor surface area relative to Day 0 for each group of animals.

FIG. 5 shows efficacy of AdbCD-D314A suicide gene therapy in intracranial human glioma xenografts. D54MG human glioma cells (0.5×10⁶ cells/mouse) were injected into the right frontal cortex of athymic nude mice (10 mice/group). Six days after tumor implantation (Day 0), a single dose of saline or 3.2×10⁷ TCID₅₀ AdCD-D314A was injected i.t. Mice then received 5 Gy fractions of radiation treatment on Days 1, 3, and 7 and 5-FC (500 mg/kg i.p. twice daily on Days 0-4, and 7-11) or saline and were subsequently monitored for survival.

FIG. 6 shows schematics of the Herpes Simplex viruses (HSV) and their parents as described. Note that since there are two copies of the g₁34,5 gene in the native virus, there are two copies of tk and CD in R3659 and M012, respectively. Also note that all the viruses contain the native viral thymidine kinase gene except for R3659, which contains a deletion at this site. As noted, all 4 viruses are deleted in both copies of the g₁34.5 gene.

FIG. 7 shows Southern blot hybridization confirms the presence of mutant bCD in MC104. DNA including shuttle plasmid pLL1pGL3-bCD, parent virus C101, and MC104 candidates were isolated, digested with PstI, and electrophoretically separated, then transferred to Zeta-Probe membrane, hybridized with pCK1037(UL3-UL4 probe). The predicted fragment sizes for each DNA are: 5.0 Kb and 1.36 Kb for pLL1pGL3-bCD; 2.09 Kb and 1.29 Kb for C101; 2.73 Kb and 2.09 Kb for MC104. MC104-309 and MC104-311 had similar results.

FIGS. 8A-8C show CD conversion results of Herpes viruses in human glioma cell lines. U87MG (FIG. 8A), D54MG (FIG. 8B), and U251 MG (FIG. 8C) cell lines were infected with 2, 0.4 and 0.04 MOI of the HSV construct M012 (expressing CD) and R3659 (control). At 24 h post-infection, conversion of 5-FC to 5-FU was determined over a 1 h time period and normalized to the amount of protein used in each assay.

FIG. 9 shows M012 replication in Neuro-2a murine neuroblastoma cells. M012 replication was compared to R3659 replication over time in murine Neuro-2a tumor cells at both high (5.0) and low (0.1) MOI. Infected cells were grown in either growth medium alone or growth medium containing 5-FC (500 μM) or 5-FU (50 μM) by determining viral titers at 12, 24, 48, 72, and 96 h post-infection in the presence or absence of 500 μM 5-FC or 50 μM 5-FU. Virus recovery at the different time points was determined by titering on Vero cells. Experiments were performed in duplicate. Inhibition was most significant at high MOI, with P=0.002.

FIG. 10 shows cytotoxicity in Neuro-2a cells. Viability studies were performed on Neuro-2a cells infected either with R3659, M012, or mock infected, and grown in the presence of 0, 100, 500, or 1,000 μM 5-FC. Percent viable cells were determined by alamarBlue assay. Absorbance values were standardized against values for wells lacking virus and drug, which represented 100% viability. The Student's t-test was used to analyze the differences between the treatment groups. For 100 μM 5-FC, M012 (MOI=0.5) P<0.001 vs. R3659 (MOI=5); for 500 μM 5-FC, M012 (both MOIs) P<0.001 vs. R3659 (both MOIs); for 1000 μM 5-FC, M012 (MOI=1.0) P<0.001 vs. R3659 (MOI=5) and P<0.032 vs. R3659 (MOI=1).

FIGS. 11A-11B show conditioned medium assay on GL261 cells. Conditioned media harvested from M012-infected Vero cells was serially diluted from 5×10⁻¹ to 5×10⁻⁶, and added to the growth medium of GL261 cells in 96-well plates. Conditioned medium was collected at 24 (a) or 48 (b) h post-infection. After 7 days at 37° C., cell viability was quantified by alamarBlue assay. Percent viability of GL261 cells is represented as a percent of the absorbance value for cells grown in media alone (no 5-FC, 5-FU or virus).

FIGS. 12A-12D show bystander killing of GL261 by 5-FU generated from 5-FC by MC104 infection of human glioma cell lines. Panel A: HSV MC104 Clone 302 was tested at 1.0 MOI against D54MG glioma cells mixed with GL261 mouse glioma cells. Panel B: HSV MC104 clone 305 vs. U87MG/GL261 mixtures; Panel C: HSV MC104 clone 305 tested vs. U251MG/GL261 mixtures; Panel D: HSV MC104 clone 309 tested versus U87MG/GL261 mixtures.

FIG. 13 shows intracranial U87MG gliomas were induced in C.B-1.7 SCID mice and 7 days later, HSV M012 or saline was injected into the tumor site. Two days later, mice were begun on twice daily intraperitoneal injections of saline (1 ml) or 5-FC (500 mg/kg). Mice were followed for survival.

FIG. 14 shows intracranial D54MG gliomas were induced in athymic nude mice and 5 days later, 1×10⁷ PFU of HSV M012 or saline (10 μl) was injected into the tumor site. Two days later, mice were begun on twice daily intraperitoneal injections of saline (1 ml) or 5-FC (500 mg/kg). Mice were followed for survival.

FIGS. 15A-15D show Immunohistochemistry staining of tumor sections for herpes simplex virus (HSV) and wild-type cytosine deaminase. U87MG flank tumors were propagated in nude mice and inoculated with 1×10⁷ PFU of M012, a γ₁34.5-deleted HSV-1 expressing the bCDwt gene under the Egr-1 promoter. Four days later, mice were killed and tumors harvested. Shown are tumor sections stained for HSV (FIG. 15A and FIG. 15B) using a polyclonal antibody against HSV-1 and HSV-2 and sections stained for bCDwt (FIG. 15C and FIG. 15D). Note the widespread tumor necrosis associated with the staining, as well as the concordance of staining for HSV and bCDwt, indicating consistent expression of the foreign gene product. (A and C, 5×; B and D, 25×.

FIGS. 16A-16F show D54MG s.c. gliomas were injected with 1×10⁷ pfu M012 HSV (FIG. 16A-16C) or M104-309 HSV (FIG. 16D-16F) and harvested 3 days later for titration of virus, measurement of CD conversion activity and immunohistochemistry. Tumors were stained with Rabbit anti-HSV (FIGS. 16A, 16B, 16D, 16E) or Rabbit anti-CD (FIGS. 16C, 16F). Panels A & D, at 4× magnification; FIGS. 16B, 16C, 16E, 16F at 10×.

FIGS. 17A-17F show D54MG gliomas were injected with 1×10⁷ pfu M012 HSV (FIG. 17A-17C) or M104-309 HSV (FIG. 17D-17F) and harvested 7 days later for titration of virus, measurement of CD conversion activity and immunohistochemistry. Tumors were stained with Rabbit anti-HSV (FIGS. 17A, 17B, 17D, 17E) or Rabbit anti-CD (FIGS. 17C, 17F). FIGS. 17A and 17D, at 4× magnification; FIGS. 17B, 17C, 17E, 17F at 10×

FIG. 18 shows the enzymatic activity of cytosine deaminase recovered from human gliomas infected with HSV M012 or MC104-309 expressing the wild-type E. coli CD or the mutant CD (D314A-CD), respectively, was measured in individual tumor homogenates at 1, 3 and 7 days after infection of the gliomas in nude mice. Data are expressed as the pmol of 5-FC converted to 5-FU per min and normalized per mg wet weight of the glioma tissue. Five mice were used per group and each tumor was assayed independently. Values represent means for 5 tumors with standard deviations indicated. The mutant CD had 3-6 fold greater conversion activity at all time points examined.

FIG. 19 shows efficacy of combination radiation and suicide gene therapy in intracranial human glioma xenografts. D54MG human glioma cells (0.5×10⁶ cells/mouse) were injected into the right frontal cortex of athymic nude mice (10 mice/group). Five days after tumor implantation, a single dose of PBS or 3.2×10⁷ TCID₅₀ AdbCD-D314A was injected i.t. on Day 5 Mice then received radiation treatment on Days 6, 9 and 13 at 2 or 5 Gy and 5-FC (500 mg/kg i.p. q2d×5/week for 3 weeks) and were subsequently monitored for survival.

FIG. 20 shows Luciferase expression in human glioma cell lines. Glioma cells, BEAS-2B normal human bronchial epithelial cells (negative control) and human HUVEC or murine endothelial cells 1P-1B and SVEC4-10 (positive control) were infected with Adflt-Luc or AdCMV-Luc (as control of infectivity) recombinant Ad at 100 MOI. Luciferase expression was analyzed at 48 h after infection by luciferase assay system (Promega). Flt-1 promoter activity is presented as a percentage of CMV promoter activity.

FIG. 21 shows RGD modification of Ad fiber knob domain increases CRAdRGDflt-1 oncolysis of glioma cells. Several glioma cell lines were infected with the CRAdflt-1 (white) or CRAdRGDflt-1 (grey) recombinant Ad at 1 MOI. Cell viability was determined at 96 h after infection by using the crystal violet inclusion assay. Data shown in comparison with uninfected control.

FIG. 22 shows CD conversion results in glioma cell lines infected with CRAdRGDflt-bCD-D341A. Cells infected with the conditionally replicative CRAdRGDflt-bCD-D341A virus at 0.5 and 0.05 MOI were tested for conversion activity 24 h post-infection. Percent conversion of ³H-5-FC to ³H-5-FU was determined over a 1 h time period.

FIG. 23 shows CD conversion in U251MG and U373MG glioma cell lines infected with CRAdRGDflt-bCD-D341A. Cells infected with the CRAdRGDflt-bCD-D341A at 0.5 MOI were tested for conversion activity at 24 and 48 h post-infection. Percent conversion of ³H-5-FC to ³H-5-FU was determined over a 1 h time period.

FIG. 24 shows CRAdRGDflt-bCD-D314A mediated oncolysis of glioma cells. Cells were infected with 0.1 MOI of CRAdRGDflt-bCD-D314A, and mock-irradiated or irradiated with 2 Gy using a ⁶⁰Co gamma irradiator one day after Ad infection. Relative cell density was determined at 5 days after radiation treatment using a crystal violet staining assay. Data shown in comparison with uninfected control cells.

FIG. 25 shows Ad-mediated molecular chemotherapy increased ionizing radiation induced increased pancreatic cancer cell death in a clonogenic survival assay. MIA PaCa-2 and Panc2.03 cells were infected with 50 MOI of AdbCDwt or AdbCD-D314A and were either mock-irradiated or irradiated at 2 Gy using a ⁶⁰Co gamma irradiator. Cells were fixed and colonies were counted at 15 days after treatment. Data are presented as percentage of colonies in comparison with untreated control. Presented are mean values±standard deviations of three independent experiments, each performed in six replicates.

FIG. 26 shows growth of Panc2.03 xenografts treated with AdbCD-D314A or AdbCDwt Treatment was started at the time of established tumor growth (Day 0 equal to 11 days after tumor cell injection). Animals were injected i.t. with 1×10⁷ TCID₅₀ of AdbCD-D314A or 1×10⁸ TCID₅₀ of AdbCDwt on Days 0, 7, and 14. 5-FC was administered i.p. at 400 mg/kg qd×5/week for 3 weeks starting on Day 1. Data points represent the mean change in tumor surface area relative to Day 0 for each group of animals.

FIG. 27 shows growth of MIA PaCa-2 xenografts treated with AdbCD-D314A alone and in combination with radiation. Treatment was started at the time of established tumor growth (Day 0 equal to 17 days after tumor cell injection). Animals were injected i.t. with PBS or 5×10⁷ TCID₅₀ of AdbCD-D314A on Days 0, 7, and 14, and then irradiated with 2 Gy on Days 1, 8 and 15 using a ⁶⁰Co gamma irradiator, and 400 mg/kg of 5-FC was i.p. administered at qd×5/week for 3 weeks starting on Day 1. Data points represent the mean change in tumor surface area relative to Day 0 for each group of animals.

FIG. 28 shows CD conversion results in glioma cell lines infected with various mutant bCD. Cells were transfected with plasmids encoding 1525 clone # 1, 1525 clone # 2 mutant bCD or bCDwt using the SuperFect Transfection Reagent (QIAGEN, Chatsworth, Calif.) and were tested 48 h post-transfection. Percent conversion of 5-FC to 5-FU was determined over a one h time period.

### DETAILED DESCRIPTION OF THE INVENTION

The present invention concerns in vivo transfection of cancer cells in solid tumors with an adenovirus encoding the cytosine deaminase gene, administration of systemic 5-FC, and radiation therapy of the tumor which resulted in tumor regression and prolonged tumor growth inhibition compared to control treatments with molecular chemotherapy or radiation therapy alone. This is the first description of how to transfect established tumors in vivo with the cytosine deaminase gene to produce enhanced therapeutic effects with the combination of molecular chemotherapy and radiation therapy. Conventional systemic administration of 5-FU produces dose limiting normal tissue toxicity. The local production of 5-FU within a tumor transfected with the cytosine deaminase gene and systemic administration of 5-FC, results in higher intratumor concentrations of 5-FU than achievable with systemic administration of 5-FU, thus improving the therapeutic ratio in combination with radiotherapy. The combination of molecular chemotherapy and radiation therapy improves treatment of a variety of cancers in humans including colon cancer, pancreatic cancer, prostate cancer, lung cancer, brain cancer, head and neck cancer and cholangiocarcinoma.

The present invention can be utilized in local and regional situations where the cancer is accessible for intratumor or regional injection of the cytosine deaminase vector. Tropism-modified adenovirus or an adenovirus encoding the cytosine deaminase gene under control of a tumor specific promoter may be required for selective gene delivery to disseminated metastatic cancer. Native adenoviral tropism can be redirected through other cell surface receptors, such as fibroblast growth factor (FGF) receptor. The present invention used targeted adenovirus to the FGF receptor as a vehicle for the delivery of cytosine deaminase to hepatobiliary tumor cells for combination of molecular chemotherapy and radiation therapy studies. The results suggest that improved gene expression may be achieved via this adenoviral-conjugate mechanism to circumvent current limitations of cancer gene therapy to solid gastrointestinal malignancies.

Thus, the present invention provides a method of treating an individual having a solid tumor, comprising the steps of treating the individual with an adenovirus encoding a cytosine deaminase gene; administering 5-FC to the individual; and treating the individual with external beam irradiation. Representative cancers treated using this method include colon cancer, pancreatic cancer, prostate cancer, lung cancer, brain cancer, head and neck cancer or cholangiocarcinoma. Preferably, the adenovirus is under control of a promoter or tumor specific promoter such as a carcinoembryonic antigen promoter, DF3/MUC1 promoter, a prostate specific antigen promoter, surfactant protein A promoter, leukoprotease inhibitor promoter, erbB-2 promoter, midkine promoter, cyclooxygenase-2 promoter, alpha fetoprotein promoter and E2F promoter.

Generally, any adenovirus encoding a cytosine deaminase gene may be used in the methods taught herein; one example is the E. coli cytosine deaminase gene. In this method, 5-FC is typically administered in a dosage of about 400-500 mg/kg twice daily and the external beam radiation is generally applied daily at a single dose of from about 2 Gy to about 3 Gy over a 4 to 6 week period. Alternatively, brachytherapy can be used as the radiation therapy. This produces greater cytotoxicity of neoplastic cells compared to treatment with adenovirus alone or external beam radiation alone.

The present invention is also directed to a method of treating an individual having a cancer, comprising the steps of combining a ligand that binds to a tumor cellular receptor and an adenoviral vector encoding a cytosine deaminase gene to form a complex; treating the individual with the complex; administering 5-FC to the individual; and treating the individual with radiation therapy. Preferably, the tumor receptor binds to the adenoviral vector. Representative cancers treated using this method include colon cancer, pancreatic cancer, prostate cancer, lung cancer, brain cancer and cholangiocarcinoma. Generally, the ligand to cellular receptor is selected from the group consisting of basic fibroblast growth factor (FGF2), epidermal growth factor and antibodies to growth factor receptors.

Preferably, the adenovirus is under control of a promoter. Generally, any adenovirus encoding a cytosine deaminase gene may be used in the methods taught herein; one example is the E. coli cytosine deaminase gene. In this method, 5-FC is typically administered in a dosage of from about 400-500 mg/kg twice daily and the external beam radiation is generally applied daily at a single dose of from about 2 Gy to about 3 Gy over a 4 to 6 week period. Alternatively, brachytherapy can be used as the radiation therapy. This produces greater cytotoxicity of neoplastic cells compared to treatment with adenovirus alone or external beam radiation alone.

The present invention further discloses a noninvasive method for continuous in vivo monitoring of 5-FU production via magnetic resonance spectroscopy (MRS). Magnetic resonance spectroscopy is capable of monitoring the biodistribution of 5-FU secondary to its ability to detect fluorine-19. Magnetic resonance spectroscopy has been able to discriminate between both the prodrug (5-FC), the active drug (5-FU) and some of the active fluorinated metabolites. The benefits of using magnetic resonance spectroscopy for detecting fluorinated compounds include the following: high detection sensitivity, low background signal, 100% natural abundance and a spin of ½.

The present invention uses magnetic resonance spectroscopy to monitor 5-FU concentrations in vivo following intratumoral injection of an adenovirus encoding the gene for cytosine deaminase and intravenous injection of 5-FC b.i.d for 5 days. Subcutaneous and metastatic pancreatic and colon cancer models are used to monitor the pharmacokinetics of 5-FU production and elimination from tumor and normal organs after transfecting these tumors with cytosine deaminase containing adenovirus.

There is a need for continuous production of 5-FU at the site of a tumor mass to maximize therapeutic efficacy and a means to detect and quantitate its concentration in tumor and in normal tissues over time in order to develop procedures that maximize 5-FU production. Magnetic resonance spectroscopy allows for monitoring this prodrug activation therapy through the following: the identification of tumor and normal tissue sites of production or accumulation of 5-FU, the discrimination of both 5-FC clearance/5-FU production, the determination of the residence time of 5-FU, the production of metabolites of the active drug, along with the determination of the elimination kinetics of 5-FU from tumor and normal organs. The information that magnetic resonance spectroscopy can provide about the pharmacokinetics of these agents can help develop procedures to maximize the effectiveness of this therapy with the potential to maximize tumor regression.

Previous studies using magnetic resonance spectroscopy did not take into account the effects of multiple dosing of the prodrug 5-FC in order to help maintain a continuous production of 5-FU or the use of multiple injections of an adenoviral vector to maximize cytosine deaminase gene transfer. Given the desire to maintain a continuous production of 5-FU, magnetic resonance spectroscopy can aid in guiding the dosing of the prodrug and the adenovirus along with monitoring the formation/elimination of 5-FU. Thus, the information that magnetic resonance spectroscopy can provide concerning the pharmacokinetics of 5-FU is valuable for development of prodrug activation gene therapy approach and provides the utility for further application to human clinical trials.

In still another embodiment of the present invention, there is provided a method of monitoring continuous conversion of 5-fluorocytosine to 5-fluorouracil in a tumor, wherein the tumor is treated with multiple doses of 5-fluorocytosine and multiple doses of adenovirus encoding a cytosine deaminase gene, comprising the steps of placing the treated tumor in a magnet; and evaluating the presence of 5-fluorocytosine and 5-fluorouracil by magnetic resonance spectroscopy over a course of time, wherein a lesser amount of 5-fluorocytosine and greater amount of 5-fluorouracil indicates increased conversion of 5-fluorocytosine to 5-fluorouracil. Preferably, the tumor is further treated with radiation.

In still yet another embodiment of the present invention, there is provided a method of monitoring continuous conversion of 5-fluorocytosine to 5-fluorouracil in a tumor, wherein the tumor is treated with multiple doses of 5-fluorocytosine and multiple doses of cytosine deaminase gene encoding adenovirus targeted by a ligand to a tumor cellular receptor, comprising the steps of placing the treated tumor in a magnet; and evaluating the presence of 5-fluorocytosine and 5-fluorouracil by magnetic resonance spectroscopy over a course of time, wherein a lesser amount of 5-fluorocytosine and a greater amount of 5-fluorouracil indicates increased conversion of 5-fluorocytosine to 5-fluorouracil. Preferably, the tumor is further treated with radiation.

In a further embodiment of the instant invention, there is provided an adenovirus encoding a cytosine deaminase gene, which selectively replicates in tumor cells. One manner in which this may be accomplished is by designing an adenovirus which has a complete E1A gene but lacks an E1B gene. The resulting adenovirus will selectively replicate in cells with a defective p53 pathway. AdE1ACD is an example of such an adenovirus.

Another embodiment of the instant invention is directed to a method of treating an individual having a solid tumor with the selectively replicating adenovirus encoding cytosine deaminase by infecting the individual with such an adenovirus, subsequently administering 5-fluorocytosine followed by radiation therapy. In yet another embodiment of the present invention, an adenovirus is provided which coexpresses cytosine deaminase and uracil phosphoribosyltransferase. Preferably the cytosine deaminase and uracil phosphoribosyltransferase are expressed as a fusion protein, such as in AdCDUPRT. Another embodiment of the instant invention is directed to a method of treating an individual having a solid tumor by administering an adenovirus coexpressing cytosine deaminase and uracil phosphoribosyltransferase followed by 5-fluorocytosine and radiation therapy.

Chemotherapy is widely used with surgery and radiotherapy for the treatment of cancer. Selectivity of most drugs for malignant cells remains elusive. The efficacy of standard chemotherapy tends to be limited by development of resistance to treatment. Unfortunately, an insufficient therapeutic index, a lack of specificity, and the emergence of radiation and drug resistant cell subpopulations often hamper the efficacy of glioma therapy [13]. A major problem for cancer treatment is the presence of toxic side effects associated with chemotherapeutic agents that limit their efficacy. There is a need for the development of new alternative therapeutic strategies. Among these approaches, gene-directed enzyme-prodrug therapy using the CD/5-FC system has been developed. In this methodology, the codA gene encoding for the CD enzyme that converts the prodrug 5-FC to 5-FU is delivered to the target tumor cells, resulting in their death. Although this approach has been in development for some time, new combinations with cancer therapies, such as selective conventional chemotherapy and radiotherapy, are being tested [2,6,14,15]. Also, to enhance efficacy of CD/5-FC molecular chemotherapy, several strategies have been developed, including irradiation of the tumor [6,16,18], use of vectors encoding the bCD:uracil phosphoribosyltransferase fusion gene,[6,19,20] or a combination of bCDwt/5-FC and herpes simplex virus thymidine kinase/ganciclovir suicide gene therapy [21,23]. Since one of the major limitations in current cancer gene therapy is the poor efficacy of in vivo gene transfer, successful application of a suicide gene will depend on its bystander killing effect. In the CD/5-FC system, the bystander effect is caused by the passive diffusion of 5-FU into the extracellular milieu and its diffusion into the adjacent cells, which requires no gap junctions [24]. In another approach, yeast CD (yCD) gene therapy has been developed. Although yCD is more efficient at converting 5-FC into the cytotoxic drug 5-FU than wild type bCD, this enzyme loses all activity by 96 h at 37° C. in contrast to bCD which retains 100% of its activity at 168 h. This loss of activity could be a critical factor in the therapeutic efficacy in vivo [25].

The present invention investigated mutant bCD gene transfer in an Adenoviral and Herpes Simplex virus directed molecular chemotherapy approach for treatment of human glioma cells in vitro and in vivo. It was been shown previously that the D314A mutation in bCD decreased efficiency for endogenous cytosine which can compete with prodrug for the active enzyme site in combination with increased efficiency for 5-FC that resulted in 19-fold relative substrate preference for 5-FC in comparison with bCDwt [11,12]. Thus, the rationale for using the mutant bCD gene was that the bCD mutant D314A would more effectively convert 5-FC to 5-FU and increase the anti-tumor activity without adverse effects.

A more potent cytotoxicity effect for human glioma cells was obtained using AdbCD-D314A/5-FC treatment in comparison with AdbCDwt/5-FC. A comparative study of AdbCD-D314A/5-FC and AdbCDwt/5-FC showed that increased cytotoxicity of mutant bCD-D314A/5-FC gene therapy correlated with significantly increased CD conversion in AdbCD-D314A treated cells. The combination of AdbCD-D314A/5-FC suicide gene therapy and radiation treatment one day after infection produced moderately increased cytotoxicity in vitro in comparison with these treatments alone and when D54MG cells were irradiated one day before or concurrent with Ad infection. However, the results of in vivo experiments demonstrated a significant delay in D54MG glioma tumor growth following AdbCD-D314A/5-FC treatment alone in comparison with AdbCDwt/5-FC alone or in combination with ionizing radiation, and when combined with radiation treatment in comparison with AdbCD-D314A/5-FC alone. These results also were confirmed using the D54MG intracranial model for survival studies. Generally, the relative response of the animal tumor models correlated with the observed in vitro proliferation and conversion assays with D54MG glioma cells. Notably, high levels of CD enzyme activity were observed for 14 days after single i.t. injection of AdbCD-D314A vector into D54MG glioma xenografts. Also, the CD conversion results demonstrated enhanced enzyme activity at 14 days after injection of tumors with AdbCD-D314A in combination with ionizing radiation one day before or after infection versus AdbCD-D314A alone (2.8 or 4,5-fold, respectively). These results are consistent with recently published data that irradiation treatment of colon cancer xenografts increased the Ad uptake and Ad mediated transgene expression in tumor cells in a dose- and time-dependent manner [26] and correlated with elevated Dynamin 2 expression [27]. These results indicate that the combination of AdbCD-D314A/5-FC suicide gene therapy with radiation treatment produced increased cytotoxicity in human glioma cells in vitro and in vivo. Combination treatment with ionizing radiation can increase expression of suicide enzymes in glioma cells, and thus produce greater cytotoxicity.

A recent study describes a conditionally replicating, Dg₁34.5 HSV (Herpes Simplex Virus) vector engineered to express bCDwt, M012, which is similar in construction to mutant oncolytic HSV already used safely for intracranial administration in humans [32]. Conditionally replicating HSV mutants that express CD have been reported by other groups. Nakamura et al. utilized an HSV mutant in which the U_{L}39 gene (encoding the ICP6 protein, or viral large ribonucleotide reductase subunit) was disrupted by the introduction of the gene for yeast CD. This vector was used for the treatment of colon carcinoma metastases of the liver, and was administered via the portal vein. However, this vector contains intact copies of the g₁34.5 gene, and thus is not safe for the treatment of malignancies arising in the brain. The instant invention has developed and extensively tested mutant Herpes simplex type 1 viruses that are conditionally replication competent. These viruses have either a deletion or truncation of both copies of the g₁34.5 gene, which prevents the virus from replicating in post-mitotic or quiescent cells or from effectively reactivating from latency. Thus, the virus is selective for tumor cells and not the post-mitotic cells of the central nervous system, which makes them suitable viral vectors for intracranial administration. The instant invention acquired or generated a panel of 21 different Dg₁34.5 HSVs expressing various foreign genes [33-39]. These Dg₁34.5 HSVs were found to be safe and effective in multiple mouse models of malignant glioma and have produced long-term survival with tumor reduction in both syngeneic and xenogeneic murine tumor models of gliomas [33, 35, 40-42]. This virus was proven safe at intracerebral doses of up to 3×10⁹ pfu in patients with malignant gliomas [42]. A Phase Ib trial at UAB conducted utilized a regimen of virus inoculation followed by tumor resection two to five days later, then reinoculation of virus into the tumor bed. The results suggest that genetically-engineered HSV is safe for inoculation into normal brain and that some replication can take place in human brain tumors. Both of these studies, as well as studies conducted in Europe using a similar HSV mutant 1716, have demonstrated the safety of these agents and have produced long-term survivors [43]. Using the HSV driven expression of the cytosine deaminase (both wild type as well as mutant), the instant invention has characterized and optimized irradiation and chemotherapeutic enhancement of molecular chemotherapy in brain tumor cell lines. Enhanced cytotoxicity was observed in vitro, following infection with the HSV encoding either wild-type or the mutant cytosine deaminase gene in presence of 5-FC. Enhanced survival was observed in a murine glioma model following infection with the HSV encoding the cytosine deaminase gene in the presence of 5-FC.

Gene therapy of cancer, in particular suicide gene therapy, has entered several clinical trials, and the factors limiting its efficacy have attracted increasing attention. Thus, target specificity, low in vivo transduction efficacy or limited cytotoxic effects are currently subjects of intense research [28,29]. The rationale behind suicide gene therapy is that after targeted transfer of these genes into tumor cells, only tumor and neighboring cells will be rendered sensitive to their cytotoxic action. As suicide gene therapy is essentially a tumor-targeted chemotherapy, the systemic toxicity commonly associated with, and a major limitation of, conventional chemotherapy is avoided. Specifically, targeted expression of the prodrug-activating enzyme avoids systemic toxicity, and results in high drug concentrations in the tumor mass and an improved therapeutic index compared to systemic drug administration. Thus, tumor-targeted suicide gene therapy is an attractive approach for human glioma therapy since local gene delivery is feasible. Also, employing a conditionally replicating Ad with selective oncolytic activity should increase therapeutic efficacy of molecular chemotherapy of glioma [30].

In one embodiment of the present invention there is provided a recombinant adenovirus vector consisting of a gene encoding a mutant cytosine deaminase operatively linked to a functional promoter; where the vector when transfected in a host, expresses cytosine deaminase in a biologically active form. The vector further comprises an arginine-glycine-aspartic acid (RGD) peptide in the fiber knob of said adenovirus. Specifically, the vector has a CMV or hTERT promoter. Moreover, the mutant cytosine deaminase gene is a E. coli gene. Further, the mutant gene harbors a substitution of an alanine for the aspartic acid at position 314 of the wild type cytosine deaminase gene. Also, the mutant gene harbors substitution of an Alanine for Valine at position 152, a Cysteine for the Phenylalanine at position 316, and Glycine for the Aspartic acid at position 317 of the wild type cytosine deaminase gene. Specifically, the adenoviral vector is a replication-deficient, adenovirus. In a related embodiment the adenoviral vector is a conditionally replicative adenovirus. Additionally, the adenovirus is under control of a tumor specific promoter. Further, the tumor specific promoter is the flt-1 promoter.

In another embodiment of the present invention there is provided a mutant Herpes Simplex Virus 1 vector consisting of a gene encoding cytosine deaminase; and a gene encoding uracil phosphoribosyl transferase operatively linked to a functional promoter; wherein said vector when transfected to a host, expresses both the cytosine deaminase and uracil phosphoribosyl transferase in a biologically active form. The genes are cistronically linked to produce a fusion protein. The mutant Herpes Simplex virus vector contains deletion in both copies of the viral g₁34.5 gene. Specifically, the promoter of the vector is selected from the group consisting of the CMV, Egr-1, TERT, FLT-1 promoter or a promoter of a gene specifically expressed in malignant cells. Additionally, the cytosine deaminase gene is a E. coli gene. In a related embodiment of the present invention the cytosine deaminase gene is mutated. Specifically, the mutant cytosine deaminase gene harbors a substitution of an alanine for the aspartic acid at position 314 of the wild type cytosine deaminase gene. Moreover, the mutant cytosine deaminase gene harbors substitution of an alanine for valine at position 152, a cysteine for the phenylalanine at position 316, and glycine for the aspartic acid at position 317 of the wild type cytosine deaminase gene. Specifically, the uracil phosphoribosyl transferase gene is an E. Coli gene.

In yet another embodiment of the present invention there is provided a method of causing selective growth inhibition of malignant tumor in a mammal consisting of introducing the genetically engineered vector of either of the compositions described supra in the mammal; where the product of the vector is expressed in the malignant tumor and administering 5-fluorocytosine, in the mammal. Additionally, this method further consists of treating the mammal with radiation therapy. Specifically, the mammal may be a human, non-human primate, cow, sheep, horse, goat, mouse, gerbil, hamster, rabbit, dog, or cat. Moreover, the tumor is selected from a group of central nervous system tumors consisting of glioma, gliosarcoma, oligodendroglioma, astrocytoma, ependymoma, primitive neuroectodermal tumor, malignant meningioma, schwannoma, malignant peripheral nerve sheath tumor or neurobalstoma. Additionally, the tumor is selected from a group consisting of malignant cells of the kidney, liver, bile duct, pancreas, lung, peritoneum, prostate, breast, uterus, skin, lips, mouth, throat, esophagus, stomach, bowel, colon and rectum. Specifically, the 5-fluorocytosine is administered in a dosage of about 12.5 to 37.5 mg/kg of body weight every six hours. Additionally, the radiation is applied at a daily dose of from about 1.8 Gy to about 2.2 Gy over a 4 to 6 week period.

In still yet another embodiment of the present invention there is provided a method of enhancing radiosensitization in a mammal in need thereof consisting of administering to the mammal a genetically engineered viral vector of either of the compositions described supra, administering 5-fluorocytosine to the mammal; and treating the individual with radiation therapy. Specifically, the mammal is a human, non-human primate, cow, sheep, horse, goat, mouse, gerbil, hamster, rabbit, dog, or cat. The mammal is suffering from a tumor from a group of central nervous system tumors consisting of, glioma, gliosarcoma, oligodendroglioma, astrocytoma, ependymoma, primitive neuroectodermal tumor, malignant meningioma, schwannoma, malignant peripheral nerve sheath tumor or neurobalstoma. In general, the mammal has a malignancy of the kidney, liver, bile duct, pancreas, lung, peritoneum, prostate, breast, uterus, skin, lips, mouth, throat, esophagus, stomach, bowel, colon and rectum. Specifically, the 5-fluorocytosine is administered in a dosage of about 12.5 to 37.5 mg/kg of body weight every six hours. Moreover, the radiation is applied at a daily dose of from about 1.8 Gy to about 2.2 Gy over a 4 to 6 week period.

Cell Culture

The human colon carcinoma cell line WiDr (ATCC CCL-218 Rockville, Md.) was grown in Earle's modified Eagle's medium (EMEM) (Gibco-BRL, Grand Island, N.Y.) supplemented with 10% fetal bovine serum (FBS) (Summit, Fort Collins, Colo.), 2 mM glutamine, and 1% non-essential amino acids in a humidified atmosphere with 5% CO₂. The human cholangiocarcinoma cell line SK-ChA-1 was the gift of A. Knuth, Ludwig Institute for Cancer Research, London, UK. SK-ChA-1 cells were maintained in RPMI-1640 medium supplemented with 2 mM L-glutamine and 10% FBS at 37° C. in a humidified 5% CO₂ atmosphere. The transformed human embryonic kidney cell line, 293, is an E1A trans-complementing cell line (Microbix, Toronto, Canada) utilized for viral propagation and titering and was maintained in Dulbecco's Modified Eagle's medium-F12 supplemented with 2 mM L-glutamine and 10% FBS at 37° C. in a humidified 5% CO₂ atmosphere. The cells were passaged using 0.05% trypsin and 5 mM EDTA once weekly.

Chemotherapeutic Drugs

5-FC (Sigma, St. Louis, Mo.) was dissolved in PBS at a stock concentration of 10 mg/ml. 5-FU (50 mg/ml, Hoffman-LaRoche, Inc., Nutley, N.J.) was used as a control for clinical therapy of both colon and cholangiocarcinoma in current medical practice.

Adenovirus Production and Characterization

The production, characterization, and functional validation of the AdCMVCD vector was described Pederson et al., Cancer Res. 57, 4325-4332, 1997; Pederson, et al., J. Gastrointestinal Surg. 2, 283-291 1998. Briefly, the cytosine deaminase gene was cloned into the adenoviral shuttle vector pACCMVpLpARS (+) (provided by R. Gerard, Katholieke Universiteit Leuven, Ontario, Canada) and then co-transfected with the pJM17 rescue plasmid (provided by Dr. F. Graham, McMaster University) into 293 cells to allow for homologous recombination. Individual plaques were isolated and subjected to 2 further rounds of plaque purification. The final adenovirus was validated by PCR and restriction analysis. The ability of AdCMVCD to induce a functional cytosine deaminase enzyme was determined by measuring conversion of ³H-5-FC to ³H-5-FU by infected cell lysate.

In Vitro Radiation Dose Response Analysis

WiDr human colon cancer cells were plated at a density of 5×10⁵ cells/well in 6-well tissue culture plates 24 hours prior to adenoviral infection. WiDr cells were then infected with AdCMVCD at a multiplicity of infection (MOI) of 1 or 10 plaque forming units (pfu) per cell in 0.5 ml Opti-Mem (Gibco-BRL) for 1.5 hours. A control virus that encodes the reporter gene E. coli LacZ which produces β-galactosidase (AdCMVLacZ) was provided by Dr. De-Chu Tang. Viral infection was stopped by the addition of 3 ml of complete growth media and the cells were returned to the incubator overnight. The following day, media was replaced with media supplemented with the appropriate concentration of 5-FC or no drug. The cells were then incubated in 5-FC for 3 days. The cells were then mock irradiated or irradiated on ice using a Picker ⁶⁰Co therapy unit (Cleveland, Ohio) at a dose rate of 80 cGy/min. The cells were then plated for colony formation. Colonies formed in 14 days and were fixed in ethanol and stained with 1% crystal violet.

SK-ChA-1 cells were infected with 10 MOI of AdCMVCD, or AdCMVLacZ, treated with 0, 10 or 20 μg/ml 5-FC for 72 hours, then irradiated with 0 or 8 Gy (80 cGy/min). The cells were irradiated on ice, then trypsinized, counted and plated in triplicate in 25 cm² tissue culture dishes (Costar) in media free of 5-FC. The plates were fixed and stained 14 days later. For both WiDr and SK-ChA-1 cells, colonies containing greater than 50 cells were counted. Percent survival was calculated as the average number of colonies counted divided by the number of cells plated times plating efficiency (PE); where PE was the fraction of colonies counted divided by cells plated without radiation. The dose response curve was fitted using the Fit v 2.4 software (provided by Dr. N. Albright, University of California at San Francisco, San Francisco, Calif.).

Animal Studies

Athymic nude mice (Frederick Cancer Research Laboratory, Bethesda, Md.) were injected s.c. in the flank with 2×10⁷ WiDr or SK-ChA-1 cells. Tumors were allowed to grow for 7 days at which time they were divided into various treatment groups. The WiDr tumor treatment groups included: 1) AdCMVCD, 5-FC and a single 10 Gy dose of ⁶⁰Co radiation; 2) AdCMVCD, 5-FC and 3×5 Gy fractions of ⁶⁰Co radiation; 3) No virus, 5-FC and 3×5 Gy fractions of ⁶⁰Co radiation; 4) AdCMVCD, 5-FC and no radiation. The AdCMVCD vector was injected intratumorally (i.t.) once every other day for a total of 3 injections beginning at Day −2 relative to radiation. The 5-FC was administered for 7 days as 500 mg/kg twice daily by i.p. injection beginning at Day −2 relative to radiation. Two days following the initial adenoviral and 5-FC injection, mice were anesthetized with ketamine-HCl (Phoenix Scientific, Inc., St. Joseph, Mo.) and irradiated. The first 5 Gy fraction was given followed by 2 subsequent 5 Gy fractions given daily. The 10 Gy single dose was given on the same day as the second 5 Gy fraction.

The SK-ChA-1 tumor treatment groups included: 1) AdCMVCD, 5-FC, and 5×2 Gy, 2) AdCMVCD, 5-FC, without radiation, 3) 5-FU (30 mg/kg/day as 15 mg/kg twice daily) without radiation, 4) 5×2 Gy radiation and 5-FU (30 mg/kg/day as 15 mg/kg twice daily), and 5) no treatment. The mice with SK-ChA-1 tumors received 5-FC (400 mg/kg twice daily by i.p. injection) beginning at Day −2 relative to radiation therapy, and continued for 7 days. The mice were anesthetized with ketamine-HCl, and their tumors irradiated using the Picker ⁶⁰Co therapy unit. All mice were shielded with a specially designed lead apparatus that allowed irradiation of a single flank (6 mice at a time). Tumor growth was measured 3 times weekly in 2 dimensions using a Vernier caliper and the tumor size (length×width) was calculated. The animals were maintained in a laminar flow room and fed sterilized chow and tap water in accordance with University of Alabama Animal Resource Department protocols.

The logrank test was used to assess if there were differences among the four groups of animals bearing WiDr xenografts in overall survival, time to tumor doubling, and time to regrowth. Specific pairwise comparisons between treatment groups for time to tumor regrowth and time to tumor doubling were also made using the logrank test. Fisher's Exact test was used to assess if there were any differences in tumor regression rate between groups.

The logrank test was used to assess if there were differences among the five groups of animals bearing SK-ChA-1 xenografts in time to tumor doubling and time to regrowth. Specific pairwise comparisons were made between treatment groups for time to tumor regrowth due to lack of an overall difference in time to tumor doubling. The level of significance used for all comparisons was P<0.05.

The ability of AdCMVCD infection combined with 5-FC to kill WiDr cells was tested. Survival was determined following AdCMVCD infection at MOI's of 1 and 10 with varying concentrations of 5-FC. Increased cytotoxicity at each MOI of AdCMVCD infection with increasing 5-FC concentration was observed in the WiDr cells. Maximal cell killing was observed at 1 and 10 MOI with administration of 20 and 4 μg/ml 5-FC, respectively. No changes in cytotoxicity were observed for the AdCMVLacZ or no virus control at the maximum tested 5-FC concentration (20 μg/ml). The survival level obtained with virus and prodrug was used to normalize for the combination radiation survival values.

Whether expression of cytosine deaminase with 5-FC treatment would enhance radiation cell killing at a single dose of radiation in WiDr cells was then determined. AdCMVCD and 5-FC concentrations giving at least 90% killing alone were used in the radiation survival experiments. Percent survival following a single 8 Gy radiation dose following AdCMVCD infection at 1 and 10 MOI with increasing 5-FC concentrations was determined for WiDr cells. Enhanced radiation cytotoxicity was observed with increasing 5-FC concentrations at each MOI tested. The maximal radiation enhanced cytotoxicity was observed at 1 and 10 MOI with 20 and 2 μg/ml 5-FC, respectively.

The conditions that gave the greatest radiosensitization at 8 Gy were identified for WiDr and used to establish a dose response relationship. The greatest increase in cell killing was observed with 1 [symbol] MOI and 20 μg/ml of 5-FC. The radiation survival curve parameters calculated using the linear quadratic and single hit multiple target (SHMT) models are listed in Table 1. Only the two AdCMVCD groups with 20 or 2 μg/ml 5-FC had non-zero α values (0.221 and 0.065 for 1 and 10 MOI, respectively). The α values were similar for all groups. For the SHMT model, the lowest D₀ values were for the AdCMVCD groups with 5-FC (0.990 and 1.034 for 1 and 10 MOI, respectively). However AdCMVCD, 10 MOI without 5-FC had a low D₀ of 1.177 compared to the range of the other groups of 1.338-1.760. Additionally, the lowest D_{q} values were obtained for the AdCMVCD groups with 5-FC (1.952 and 2.569 for 1 and 10 MOI, respectively) while the values for the other groups ranged from 3.207-3.825.

TABLE 1
R Radiobiologic parameters of in vitro survival curves for
human colon cancer cell line WiDr infected with Ad CMVCD,
AdCMVLacZ or no viral infection, treated with 5-FC and
exposed to ⁶⁰Co radiation.

| | Linear Quadratic | | | | Single Hit Multiple | | |
|---|---|---|---|---|---|---|---|
| | Parameters | | | | Target Param [symbol] | | |
| Treatment Group | a | b | al | r² | Dₒ | D_{q} | r² |
| No Viral Infection, 0 mg/ml 5-F [symbol] | 0 | 0.05 | 0 | 0.993 | 1.338 | 3.825 | 0.999 |
| No Viral Infection, 20 mg/ml 5- [symbol] | 0 | 0.04 | 0 | 0.999 | 1.760 | 3.679 | 0.998 |
| A AdCMVLacZ, 10 MOI, 0 mg/ml [symbol] | 0 | 0.04 | 0 | 0.996 | 1.502 | 3.614 | 0.993 |
| AdCMVLacZ, 10 MOI, 20 mg/ml [symbol] | 0 | 0.04 | 0 | 1.00 | 1.673 | 3.459 | 0.999 |
| AdCMVCD, 1 MOI, 0 mg/ml 5- [symbol] | 0 | 0.05 | 0 | 1.00 | 1.496 | 3.207 | 0.999 |
| AdCMVCD, 1 MOI, 20 mg/ml 5- [symbol] | 0.2 | 0.06 | 3.2 | 0.979 | 0.990 | 1.952 | 0.996 |
| AdCMVCD, 10 MOI, 0 mg/ml 5- [symbol] | 0 | 0.05 | 0 | 0.988 | 1.177 | 3.674 | 1.00 |
| AdCMVCD, 10 MOI, 2 mg/ml 5- [symbol] | 0.0 | 0.07 | 0.8 | 1.00 | 1.034 | 2.569 | 0.998 |

The radiation induced killing of SK-ChA-1 cells treated with AdCMVCD infection, 5-FC, and 8 Gy radiation. The enhanced effects of combined treatment were most evident at the 20 μg/ml dose of 5-FC. A detailed radiation dose response analysis was published in Table 1 of Pederson et al. These prior studies demonstrated a D₀=0.968 and α=0.444 for the combined modality treatment of the SK-ChA-1 cholangiocarcinoma cells. The large value of α and small D₀ indicate significant reduction in cell survival as a result of the combined treatments with low (2 Gy) and high (8 Gy) single fraction radiation exposures. A similar trend was observed in the radiation survival parameters obtained using the WiDr colon cancer cells (Table 1). The largest a values and smallest D₀ values were observed for the AdCMVCD infected cells treated with 5-FC.

To establish the efficacy of cytosine deaminase and 5-FC with radiation therapy for WiDr cells in vivo, subcutaneous WiDr tumors were established in the flanks of athymic nude mice. The irradiation conditions included a single 10 Gy dose or 3×5 Gy fractions on 3 consecutive days. Two mice from each combination therapy group died from the treatment. Tumor growth was measured and the change in tumor size determined over time. Two of 6 tumors in the combined AdCMVCD+5-FC+3×5 Gy modality group regressed but subsequently recurred, while 3 of 6 tumors regressed then recurred in the Gy combined modality group. The AdCMVCD+5-FC+10 Gy and the AdCMVCD+5-FC+3×5 Gy groups produced the longest times to tumor regrowth and tumor doubling, but were not significantly different from each other. The AdCMVCD+5-FC+10 Gy, AdCMVCD+5-FC+3×5 Gy and the 5-FC+3×5 Gy groups all had significantly longer times to tumor doubling than the AdCMVCD+5-FC+0 Gy group (P=0.0037, 0.01, and 0.0006, respectively) as well as significantly longer times to tumor regrowth (P=0.001, 0.0026, and 0.001, respectively). Both the AdCMVCD+5-FC+10 Gy and the AdCMVCD+5-FC+3×5 Gy groups had significantly longer times to tumor regrowth than the 5-FC+3×5 Gy group (P=0.0103 and 0.0153, respectively). The 5-FC+3×5 Gy and the AdCMVCD+5-FC treated groups were not significantly different. No significant pairwise differences existed in time to tumor regrowth or doubling. Tumor growth was inhibited for a longer period with AdCMVCD+5-FC+5×5 Gy.

The AdCMVCD+5-FC+5×2 Gy and the 5-FU+5×2 Gy groups had the longest times to SK-ChA-1 tumor regrowth, however they were not significantly different from each other. No differences existed in time to tumor doubling among the treatment groups. The time to tumor regrowth did not differ between the AdCMVCD+5-FC and 5-FU alone treatment groups. The AdCMVCD+5-FC+5×2 Gy group had a significantly longer time to tumor regrowth compared to the 5-FU alone and the AdCMVCD+5×2 Gy groups (P=0.0126 and 0.0121, respectively). The 5-FU+5×2 Gy group also had a significantly longer time to tumor regrowth compared to the 5-FU alone and AdCMVCD+5×2 Gy groups (P=0.0204 and 0.0180, respectively).

The use of gene transfer methods employing adenoviral vectors to sensitize cells to the effects of ionizing radiation can be used for solid tumor therapy. An adenovirus encoding the cytosine deaminase gene used with the prodrug 5-FC can lead to enhanced cell killing when used in combination with ionizing radiation in vitro and in vivo for 2 human gastrointestinal malignancies, colon carcinoma and cholangiocarcinoma. Studies in human cholangiocarcinoma demonstrated the in vitro radiosensitizing effects of combining cytosine deaminase transgene expression with 5-FC prodrug treatment and single fraction radiation therapy. The small D₀ and large a values obtained for the combination treatment groups indicate cytotoxic effects both at high and low radiation doses for the WiDr cells which is similar to what occurred with the cholangiocarcinoma cells.

From the encouraging results of the in vitro evaluation of induction of radiosensitivity for both colon cancer and cholangiocarcinoma cells, in vivo models were evaluated. For SK-ChA-1 cholangiocarcinoma tumors, an enhanced anti-tumor effect was seen from combined AdCMVCD infection, 5-FC administration, and a single 10 Gy radiation dose compared to AdCMVCD infection and 5-FC alone. Radiation therapy in the clinical setting is traditionally delivered in daily 2 Gy doses over 4-6 weeks. Analysis of this format of radiation therapy and CD/5-FC gene therapy with human colon cancer and cholangiocarcinoma indicated the fractionated delivery of 3×5 Gy doses or 5×2 Gy doses was at least as effective as a single 10 Gy fraction or with systemically administered 5-FU and 5×2 Gy doses. Thus, a measurable anti-tumor effect was observed with CD/5-FC gene therapy in combination with low dose fractionated radiation therapy.

Khil et al. showed that the cytosine deaminase gene stably transfected into WiDr cells was able to enhance radiation cell killing in vitro. Adenoviral vectors have been used in many gene transfer and therapy studies. The use of adenoviral vectors to encode cytosine deaminase and convert 5-FC to 5-FU to achieve cell killing has been reported. Ohwada et al. delivered an adenoviral vector encoding cytosine deaminase into normal tissue 0.8-1 cm from the site of colon tumor xenografts in the liver of mice and systemically delivered 5-FC to suppress metastatic tumor growth. Therefore, there is potential that treatment of primary tumor nodules with the combination of 5-FC conversion to 5-FU by the cytosine deaminase gene and radiation could lead to increased local control while the production of 5-FU would serve to suppress metastatic growth.

Both metastatic colon carcinoma and locally advanced cholangiocarcinoma are difficult clinical problems, and have been resistant to single modality therapy. The gene therapy approach of molecular chemotherapy combined with radiation therapy provides a new approach to the treatment of solid tumors. The ability of 5-FC and 5-FU to freely diffuse across cell membranes is one advantage of the CD/5-FC toxin gene/prodrug strategy. This is in contrast to the HSVtk/GCV system where cellular gap junctions are a vital component of the bystander effect. Another advantage of CD/5-FC demonstrated with respect to the WiDr colon cancer model was efficacy at a very low viral concentration of 1 MOI. A MOI of 1 with a high 5-FC concentration (20 μg/ml) was more effective than a MOI of 10 and a low 5-FC concentration (2 μg/ml). This is an important observation since it may be difficult to achieve 100% infection of cells in solid tumors in situ. In vivo studies lend support to the possibility that less than 100% tumor infection can be effective. Although it is likely that only a fraction of tumor cells in the xenografts were infected, a significant regrowth delay was observed in the irradiated, AdCMVCD infected tumors treated with 5-FC compared to irradiation alone or the AdCMVCD infected and 5-FC treated tumors without irradiation. An important observation was that low dose multifraction radiation treatment in combination with CD/5-FC gene therapy was effective in inhibiting tumor growth.

Such enzyme/prodrug strategy consisting of CD/5-FC relies on diffusion of the cytotoxic enzymatic product 5-FU to kill non-transduced tumor cells. It can be utilized in local and regional situations where the cancer is accessible for intratumor or regional injection of the cytosine deaminase vector. Tropism-modified adenovirus or an adenovirus encoding the cytosine deaminase gene under control of a tumor specific promoter may be required for selective gene delivery to disseminated metastatic cancer. However, native adenoviral tropism can be redirected through other cell surface receptors, such as fibroblast growth factor (FGF) receptor. The following examples demonstrate methods to increase gene delivery via vector binding to tumor markers. Adenovirus vector was redirected via FGF receptor for the delivery of cytosine deaminase gene to hepatobiliary tumor cells for combination of molecular chemotherapy and radiation therapy studies.

Cell Lines

The human cholangiocarcinoma cell lines SK-ChA-1 and Oz were from Dr. A. Knuth (Ludwig Institute for Cancer Research, London, UK) and Dr. N. F. LaRusso (Mayo Clinic, Rochester Minn.) respectively. BXPC-3, ASPC-1 and CFPAC-1 human pancreatic carcinoma cell lines were obtained from the American Type Culture Collection (ATCC CRL-1687, ATCC CRL-1682 and ATCC CRL-1918; Rockville Md.). SK-ChA-1, Oz and BXPC-3 cells were maintained in RPMI-1640 medium supplemented with L-glutamine (2 mM), and 10% heat inactivated fetal bovine serum (FBS) (Summit Biotechnology, Ft. Collins, Colo.) at 37° C. in a humidified 5% CO₂ atmosphere. ASPC-1 cells were maintained in RPMI-1640 medium supplemented with L-glutamine (2 mM), and 20% FBS at 37° C. in 5% CO₂ atmosphere.

Fab-FGF2 and Fab′-FGF2 Conjugates

The recombinant adenoviral vectors (AdCMVLacZ, AdCMVLuc, and AdCMVCD) were redirected with FGF2 to the FGF receptor by utilization of a bi-specific conjugate constructed and validated as described (Paillard, F., Human Gene Ther. 8, 1733-1736, 1997). Fab-FGF2 was constructed by utilizing the 1D6.14 anti-adenoviral knob monoclonal antibody, and production of the Fab fragment. This moiety was conjugated to human FGF2 by disulfide linkage.

To decrease the heterogeneity of the Fab-FGF2 conjugates, a Fab′-FGF2 conjugate was generated. The ascites containing the anti-knob 1D6.14 antibody was loaded onto a protein A column in phosphate buffer, pH 7.4 and eluted with 0.1 M glycine pH 3.5. The purified IgG was digested with immobilized pepsin to obtain F(ab)′₂ fragments. The digestion mixture was purified by protein A chromatography and the flow-through containing the F(ab)′₂ was buffer exchanged by gel filtration chromatography (Sephacryl S-200, Pharmacia, Uppsala, Sweden). The purified F(ab)′₂ fragments were mildly reduced with 2-mercaptoethylamine-HCl. The sulfhydryl group on the Fab′ fragment was activated with Ellman's reagent (DTNB) at a 1:3 molar ratio for 30 min which results in Fab′-TNB. Excess DTNB was removed by diafiltration using an Amicon stirred cell apparatus (Beverly, Mass.) equipped with a YM30 and then put through a 0.2 μm filter to obtain pure TNB-Fab′. TNB-Fab′ and FGF2 were mixed at a 1:1 molar ratio and incubated for 12-16 hours at 4° C. to generate the Fab′-FGF2 conjugate. The reaction mixture was purified by heparin affinity chromatography (Heparin Sepharose, FF, Uppsala, Sweden).

Fractions containing Fab′-FGF2 were further purified by gel filtration (Sephacryl S-100 HR, Pharmacia, Uppsala, Sweden). The Fab′-FGF2 was filtered through a 0.2 μm membrane and stored at −80° C. The material was determined to be greater than 95% pure by SE-HPLC. The Fab′-FGF2 conjugate was analyzed using the anti-knob ELISA and shown to have very similar binding characteristics as the anti-knob Fab and Fab-FGF2. In addition, the materials, final product and intermediates were characterized by SDS-PAGE under reducing and non-reducing conditions. All the materials migrated as expected and the final product was pure.

Functional validation of the conjugate moieties was defined prior to use. The Fab and Fab′ moiety binding to adenoviral type 5 knob protein was confirmed by ELISA. Functional ability of the FGF2 moiety of the conjugate was evaluated using a bovine aortic endothelial cell proliferation assay. The Fab-FGF2 and Fab′-FGF2, when complexed with Ad5, showed comparable levels of gene expression when assayed on SKOV3.ip1 cells. Fab-FGF2 was used in the majority of the in vitro studies and the in vivo study utilized the Fab′-FGF2 as the retargeting moiety.

Recombinant Adenoviruses

E1A deficient replication-incompetent serotype 5 adenoviral vectors were used to analyze Fab-FGF2 and Fab′-FGF2 redirected adenoviral gene transfer. AdCMVLuc encodes the firefly luciferase gene under the control of the human cytomegalovirus (CMV) promoter/enhancer, and has been described.

AdCMVLacZ contains the LacZ reporter gene and induces expression of the E. coli β-galactosidase enzyme under control of the CMV promoter. AdCMVCD encodes the E. coli cytosine deaminase gene under control of the CMV promoter, and was constructed, functionally validated, and propagated.

Redirected Marker Gene Adenoviral Infections

Either AdCMVLuc or AdCMVLacZ was incubated with Fab-FGF2 conjugate in a volume of 130 μl at room temperature for 30 minutes. Dilutions of this stock to varying plaque forming units (pfu) of virus were made and then added to 30,000 cells/well in a 12 well dish (Costar, Cambridge, Mass.) and incubated at 37° C. for 2 hours. Infections were terminated by addition of 5 ml of complete media.

Analysis of AdCMVLuc and AdCMVLacZ Gene Expression

Luciferase assays were performed according to the manufacturer's instructions 24 hours after infection (Luciferase Assay Kit, Promega, Madison, Wis.). Briefly, cell lysates from infected cells were obtained by aspirating culture media, washing cells with PBS, and adding 150 μl of cell lysis buffer to each well. Cells were lysed at room temperature for 10 minutes and cellular debris removed by refrigerated centrifugation at 13,000×g for 5 minutes. Assay reagent was added to the cell lysates and analyzed for emitted light on a luminometer (Lumat, Berthold, Nashua, N.H.).

To analyze AdCMVLacZ gene expression, in brief, 48 hours following infection cells were fixed in 12-well dishes (Costar) with 0.5% glutaraldehyde (Sigma). The cells were washed with PBS, and stained with X-gal 5-bromo-4-chloro-3-indolyl-β-D-galactoside substrate with 2 mM MgCl₂, 5 mM K₃Fe(CN)₆, and 0.3% Nonidet P-40 (Sigma).

In Vitro Adenoviral Infections for Fab-FGF2 Redirection of Reporter Gene Expression and Measurement of Reporter Gene Expression

Cells were plated at a density of 4×10⁴ per well in 12-well culture dishes and infected with recombinant adenovirus (AdCMVLacZ or AdCMVLuc) or adenovirus+Fab-FGF2 conjugate 24 hours later. The adenovirus and Fab-FGF2 conjugate were mixed in a volume of 130 μl at room temperature, and allowed to incubate for 30 minutes prior to infection of the cell monolayers. Cellular infections were carried out in a minimal volume (0.5 ml) of Optimem (Gibco BRL, Grand Island, N.Y.) for 2 hours at 37° C., then 5 ml of complete medium added.

The luciferase kit from Promega was used according to manufacturer's recommendations. Cells were lysed, and the cell lysates assayed for luciferase activity using a Berthold luminometer (Nashua, N.H.). Bradford protein assay was used to quantitate the protein in the samples. The data is reported as relative light units (RLU)/μg protein and is the average of 3 independent experiments.

Detection of Cytosine Deaminase Protein in AdCMVCD and AdCMVCD+Fab-FGF2 Infected Cells

Five cell lines, SK-ChA-1, BXPC-3, Oz, CFPAC-1 and ASPC-1 were transfected as described with various MOI AdCMVCD and AdCMVCD+Fab-FGF2. Proteins were isolated from cells using Triton X-100 solubilization buffer (1% Triton X-100, 50 mM Hepes pH 7.5, 150 mM NaCl, 1.5 mM MgCl₂, 1 mM EDTA, 200 μM sodium orthovanadate, 10 mM sodium pyrophosphate, 100 mM sodium fluoride, 1 mM PMSF, 10% glycerol). Proteolytic inhibitors were added (aprotinin and leupeptin at a concentration of 10 μg/ml). The sample preparations with isolation buffer were incubated 10 minutes on ice, microfuged at 12,000×g for 15 minutes at 4° C., and the supernatant was collected.

Cytosine deaminase was separated by sodium dodecylsulphate-polyacrylamide gel electrophoresis (SDS-PAGE) as described by Laemmli and the samples were run under reducing conditions. Protein concentrations of the solubilized preparation were determined using the Pierce BCA protein assay kit and equal concentrations of total protein were loaded onto each lane of the gel. Rainbow colored protein weight markers (Amersham, Arlington Heights, Ill.) also were loaded onto one lane of each gel.

Proteins were electro-transferred to nitrocellulose membranes as described by Towbin et al. for 12-15 hours at 0.1 amp and 1 hour at 1 amp. Membranes were placed in milk block buffer pH 7.5 (10% powdered mild, 0.02% Nonidet P-40, 0.15 M NaCl, 0.02 M Tris) overnight at 4° C. Membranes were then incubated overnight at 4° C. with a monoclonal antibody specific for CD (37) at 5 μg/ml. The blots were rinsed, and a goat anti-mouse IgG conjugated to alkaline phosphatase was added at a concentration of 0.5 μg/ml for 1 hour to bind the primary antibody. After rinsing, an alkaline phosphatase color development kit (BioRad, Hercules, Calif.) was used to visualize the antigen-antibody reaction.

In Vitro Evaluation of AdCMVCD vs. AdCMVCD+Fab-FGF2 Mediated Cellular Cytotoxicity

SK-ChA-1 and BXPC-3 cells were plated at 1.5×10⁶ cells per well in 6-well plates and infected 24 hours later at a confluency of 80% with AdCMVLacZ, AdCMVCD or AdCMVCD+Fab-FGF2. Twenty-four hours later, cells were trypsinized, counted, and plated (5,000 cells/well) in 96-well microtiter plates (Costar) in 6 replicates. Media was supplemented with 5 μg/ml 5-FC (Sigma). Cell proliferation was determined by colorimetric assay (CellTiter 96 AQueous non-radioactive cell proliferation assay kit, Promega) after various periods of incubation. This assay measures the conversion of a tetrazolium salt (MTS) to formazan by viable cells. The absorbance at 490 nm was then measured in a 96-well plate reader (Molecular Devices, Menlo Park, Calif.). Data collected by the plate reader was analyzed by the SOFTmax software package (Emax Molecular Devices, Menlo Park, Calif.).

Effectiveness of AdCMVCD vs. AdCMVCD+Fab-FGF2 Induction of CD Expression

The relative expression of functional cytosine deaminase enzyme and its conversion of [6-3H]-5-FC to [6-³H]-5-FU was evaluated for cells infected with AdCMVCD or AdCMVCD+Fab-FGF2 using a modification of the procedure described by Haberkorn et al. SK-ChA-1 or BXPC-3 cells were plated at 1.5×10⁶ cells per well in 6-well plates (Costar) and infected 24 hours later at a confluency of 80% with AdCMVLacZ, AdCMVCD, and AdCMVCD+Fab-FGF2 at various viral plaque forming units (pfu). Twenty-four hours later, cells were harvested and lysed by 4 freeze-thaw cycles in 100 mM Tris-HCl, 1 mM EDTA/dithiothreitol (Sigma), pH 7.8. Cellular debris was pelleted by centrifugation at 14,000 rpm for 5 minutes. The cytosolic fraction was separated, and 6-10 μg of cytosolic protein was incubated with (0.5 μCi) [6-³H]-5-FC (Sigma) at 37° C. for 6 hours. Each reaction mixture plus 5-FU and 5-FC standards were then spotted on a cellulose thin layer chromatography plate (Eastman Kodak, Rochester, N.Y.) and developed in a butanol-water chamber. Each region (5-FU and 5-FC) was visualized under UV light, and respective areas cut from the plate and placed in 5 ml EcoLume scintillation fluid (ICN, Costa Mesa, Calif.). Each region was counted for radioactivity in a Packard Tri-Carb 1900 TR liquid scintillation counter. The [³H] gate (0-18.6 keV) was utilized, with a counting efficiency of 60%. Percent conversion of 5-FC to 5-FU was calculated as activity in the 5-FU fraction compared to the total counts in the 5-FC and 5-FU fractions for each treatment condition.

AdCMVCD vs. AdCMVCD+Fab′-FGF2 in Combination with 5-FC Prodrug Administration and External Beam Radiotherapy for Induction of Anti-Tumor Response

The utility of FGF2 retargeting of AdCMVCD for augmentation in efficacy of this approach was evaluated. Female athymic nude mice (National Cancer Institute Frederick Research Laboratory, Frederick, Md.) were injected s.c. with 2×10⁷ BXPC-3 cells in 50 μl PBS in both flanks. Tumors with diameters of 7 to 10 mm developed in 7 days. On Day −2 relative to radiation treatment, right sided tumors were injected with AdCMVCD+Fab′-FGF2 at 2×10⁷ pfu in a 50 μl volume, using a 27-gauge needle, and left sided tumors were injected with 2×10⁷ pfu of AdCMVCD. Animals were administered 5-FC at 400 mg/kg twice daily by i.p. injection beginning on Day −2 relative to radiation and continuing for 7 days. On Day 0, animals were anesthetized with 2 mg ketamine (Phoenix Scientific Inc., St. Joseph, Mo.) by i.p. injection and treated with 5 Gy ⁶⁰Co radiation (80 cGy/min) with a Picker C-9 80 cm isocenter clinical irradiator (Cleveland, Ohio). Tumor diameters were measured blinded with a Vernier caliper 3 times weekly and the surface area (product of length×width) calculated. Animals were maintained in a laminar flow room under sterile conditions and fed sterilized mouse chow and tap water in accordance with University of Alabama Animal Research guidelines.

A two factor analysis of variance with interaction was used to assess the effects of AdCMVLuc and AdCMVLuc+Fab-FGF2 MOI on RLU for each of the cell types individually. Due to the nonconstant variability, the logarithm of RLU was analyzed. This transformation stabilized the variability and normalized the errors. A three-factor with interaction analysis of variance was used to assess the effects of MOI, virus type and day on the number of cells per well. Due to the nonconstant variability, the logarithm of cells per well was analyzed. This transformation stabilized the variability and normalized the errors. Global comparisons were done at the 5% significance level and all pairwise comparisons were done at the 1% significance level. A nonlinear model was used to calculate the 5-FU IC₅₀ for each cell type individually. The nonlinear model is given by: number of cells=trough+(peak-trough)/(1+dose/IC₅₀). To assess the correlation of the number of cells per well with 5-FU production, a simple linear regression was done modeling the logarithm of cells per well as a function of percent 5-FU production for each cell and virus type combination individually. Kaplan-Meier estimates on the difference in time to tumor size doubling was used to assess the difference in tumor growth in animals treated with AdCMVCD or AdCMVCD+Fab′-FGF2 plus 5-FC and radiation.

Determination of Firefly Luciferase Expression in Pancreatic and Cholangiocarcinoma Cells with a Redirected Adenoviral Vector

Analysis of infectivity of SK-ChA-1 cholangiocarcinoma cells and BXPC-3 pancreatic carcinoma cells mediated by Fab-FGF2 redirection of AdCMVLuc was performed, and the results are shown. SK-ChA-1 and BXPC-3 cells were transduced with 1, 10 and 100 MOI AdCMVLuc. An order of magnitude improvement in luciferase gene expression was observed with redirection of AdCMVLuc infection with Fab-FGF2 in SK-ChA-1 cholangiocarcinoma cells and BXPC-3 pancreatic carcinoma cells. This augmented gene delivery was blocked by pre-incubation of the conjugated virus with excess anti-FGF2 antibody. Thus, the results demonstrate increased adenovirus mediated gene delivery via the FGF2 ligand to these cells.

Determination of E. coli β-galactosidase Expression in Pancreatic and Cholangiocarcinoma Cells with a Redirected Adenoviral Vector

An analysis was undertaken to visually demonstrate differential β-galactosidase expression in BXPC-3 and SK-ChA-1 cells following infection with AdCMVLacZ and AdCMVLacZ+Fab-FGF2. X-gal staining of both cell types indicated improved transduction efficiency of cells when AdCMVLacZ infection was redirected via Fab-FGF2. LacZ gene expression was inhibited by pre-incubation of BXPC-3 and SK-ChA-1 cells with 25 μg heparin. This improved redirected transduction efficiency of cells correlated with greater luciferase gene expression.

5-FU Cytotoxicity to Pancreatic and Cholangiocarcinoma Cell Lines

To determine the relative sensitivity of several hepatobiliary cell lines to 5-FU, the concentration of 5-FU which inhibited cellular growth by 50% (IC₅₀) was determined. Characterization of this information is particularly relevant to the CD/5-FC toxin gene prodrug system, as 5-FU is the toxic metabolic product of cytosine deaminase enzymatic conversion of 5-FC. The most 5-FU sensitive cell lines were CFPAC-1 and SK-ChA-1 with IC₅₀ values of 0.089 μg/ml and 0.115 μg/ml, respectively. BXPC-3 and ASPC-1 were less sensitive with IC₅₀ values of 0.134 μg/ml and 0.635 μg/ml, respectively. Thus, these results indicate that for equivalent levels of cytosine deaminase gene transfer, the various cell lines should show a differential level of 5-FU mediated cytotoxicity.

Determination of Differential Expression of Cytosine Deaminase in AdCMVCD and AdCMVCD+Fab-FGF2 Infected Pancreatic and Cholangiocarcinoma Cells

Western blotting of cell lines transfected at various MOIs demonstrated that in all cell lines, the retargeted transfection with AdCMVCD+Fab-FGF2 resulted in a greater cellular concentration of cytosine deaminase protein than transfection with AdCMVCD alone. At a higher MOI in SK-ChA-1 retargeting generated a greater concentration of cytosine deaminase protein.

Differential Cytosine Deaminase Function in Pancreatic and Cholangiocarcinoma Cells Based upon FGF2 Redirection

The relative conversion of 5-FC into 5-FU for selected hepatobiliary cell lines infected with AdCMVCD or AdCMVCD+Fab-FGF2 is shown. The highest ³H-5-FC conversion to ³H-5-FU after cellular infection with 10 MOI of AdCMVCD was seen in SK-ChA-1 (44.4%), and BXPC-3 (38.4%) cells. A lower level of conversion was seen in CFPAC-1 (6.4%) pancreatic carcinoma cells. When AdCMVCD infection was redirected by pre-incubation with Fab-FGF2, higher levels of CD enzymatic activity were observed at 10 MOI for SK-ChA-1 cells (93.5%) and BXPC-3 cells (74.8%). Similar trends were noted at 5 MOI in SK-ChA-1 cells and 1 or 2 MOI in BXPC-3 cells. For CFPAC-1 cells, increased 5-FU production via Fab-FGF2 redirection of AdCMVCD was seen in the 100 MOI group (66.9%). Cytosine deaminase mediated conversion of ³H-5-FC into ³H-5-FU was inhibited to less than 10% when AdCMVCD+Fab-FGF2 was preincubated with 25 μg heparin. Control conditions of no viral infection or control AdCMVLacZ viral infection did not result in 5-FU production, and had background levels of radioactivity (<10%). Thus, the level of 5-FC to 5-FU conversion following adenoviral retargeting compared to native adenovirus was highest at low MOI in SK-ChA-1 and BXPC-3 cell lines, while the 5-FC to 5-FU conversion rate in CFPAC-1 cells following adenoviral retargeting was high only at high MOI.

Determination of Differential Cytotoxicity in Pancreatic and Cholangiocarcinoma Cells Based upon FGF2 Redirection of AdCMVCD

The ability of adenoviral vector redirection to enhance the sensitivity of tumors in the context of the CD/5-FC approach was evaluated. Cytotoxicity to hepatobiliary cells transduced by AdCMVCD or AdCMVCD+Fab-FGF2 and exposed to 5 μg/ml 5-FC is shown. The greatest cytotoxic effects of AdCMVCD (10 MOI) were observed in SK-ChA-1 and BXPC-3 cells. These results are consistent with the 5-FU production data. SK-ChA-1 cells infected with 5, 10, or 100 MOI AdCMVCD and exposed to 5-FC for 7 days had 41.4%, 26.8% and 11.7% of cells/well relative to cells infected with 100 MOI AdCMVLacZ and exposed to 5-FC for 7 days. BXPC-3 cells infected with 1, 2, or 10 MOI AdCMVCD and exposed to 5-FC for 6 days had 73.3%, 47.7% and 19.9% of cells/well relative to cells infected with 100 MOI AdCMVLacZ and exposed to 5-FC for 6 days. No increase in cytotoxicity was observed in the CFPAC-1 cells with increasing MOI of AdCMVCD from 5 to 100. The cell lines which converted significant amounts of 5-FC into 5-FU, had the greatest cytotoxicity following infection with AdCMVCD and exposure to 5 μg/ml 5-FC.

Cytotoxic effects induced by AdCMVCD were enhanced by pre-incubation of AdCMVCD with Fab-FGF2 prior to infection of the cells. SK-ChA-1 cells infected with 5, 10 and 100 MOI AdCMVCD+Fab-FGF2 and exposed to 5-FC for 7 days had significantly more toxicity relative to cells infected with 5, 10 and 100 MOI AdCMVCD and exposed to 5-FC for 7 days (p=0.0001, 0.0001 and 0.0001, respectively). In BXPC-3 cells following infection with 10 or 100 MOI of AdCMVCD+Fab-FGF2 compared to AdCMVCD, retargeting with Fab-FGF2 did not result in differential cytotoxicity. The overall level of cell killing was significantly greater than no treatment controls. As BXPC-3 cells were shown to be relatively sensitive to 5-FU mediated killing with an IC₅₀ value of 0.134 μg/ml, the dose of AdCMVCD was decreased to 1 and 2 MOI in these cells. BXPC-3 cells transduced at these lower MOIs resulted in a differential cytotoxic effect between AdCMVCD and AdCMVCD+Fab-FGF2 infected BXPC-3 cells (p=0.0001). In contrast, CFPAC-1 cells which did not readily convert 5-FC to 5-FU, showed induction of cytotoxicity only at 100 MOI AdCMVCD+Fab-FGF2 (p=0.0001). This data correlates with 5-FU production data.

In Vivo Determination of Therapeutic Efficacy of Multimodality Therapy for Pancreatic Tumors Utilizing Fab′-FGF2 Redirected AdCMVCD, 5-FC Administration, and External Beam Radiotherapy

For BXPC-3 tumors injected with AdCMVCD+Fab′-FGF2, systemic 5-FC, and external beam radiotherapy, the time to tumor doubling was extended compared to tumors injected with AdCMVCD, systemic 5-FC, and external beam radiotherapy. Based on the 95% confidence interval, adenoviral retargeting extended the time to tumor doubling by 1 to 28 days.

Tumor Cell Conversion of 5-FC to 5-FU via MRS Over Time in Human Pancreatic and Colon Cancer Cells Both In Vitro and In Vivo

In vitro models of molecular chemotherapy were developed using the AdCMVCD cytosine deaminase toxin gene for the transfection of both human pancreatic and colon cancer cell lines (BXPC-3, WiDR, LS174T). These lines when transfected with the AdCMVCD have demonstrated >75-80% cytotoxicity after the addition of 5-FC to the media. In this study, an in vitro treatment model was proposed using 3×10⁶ BXPC-3 pancreatic tumor cells transfected at a MOI of 100 with AdCMVCD. These cells were then examined by MRS at 0, 2, 6 and 24 hours for conversion of 5-FC to 5-FU.

Initial studies have shown the sensitivity of fluorine spectroscopy by its ability to detect 3.8 mM 5-FC when initially added to transfected BXPC-3 cells. Initial data shows that after 90 minutes, no significant change in signal intensity of the 5-FC was identified with minimal 5-FU detected. However, after 120 minutes, the signal for 5-FC had dropped to 50% of its original intensity and the signal for 5-FU had increased to approximately 40% of the original 5-FC signal indicating conversion of 5-FC to 5-FU.

Optimize the Conditions to Achieve Prolonged Maximal Production of 5-FU in Mouse Tumor Models Monitored by MRS

MRS was used to optimize the prodrug approach using mouse tumor models. Metastatic hepatic tumor models of colon and pancreatic cancer were developed. Special delivery procedures for adenovirus and the delivery of the prodrug were proposed. Pancreatic and colon tumors were grown both subcutaneously and in the liver following intrasplenic injection and the tumors were transduced with cytosine deaminase containing adenovirus. The adenovirus will be targeted to the tumors in the liver via basic fibroblast growth factor. The animals and tumors will then be subjected to varying dosing schedules of the prodrug, to varying amounts of radiation and to multiple doses of the adenovirus. MRS allows a continuous in vivo detection system for 5-FU during these treatment conditions in the same animal over time. Through this, each mouse will be monitored over time and the pharmacokinetics measured of the prodrug 5-FC, the active drug 5-FU, along with monitoring which combination of procedures produces the greatest inhibition of tumor growth. It is expected that the use of MRS can help maximize the tumoricidal properties of CD/5-FC gene therapy and that planned human trials will also incorporate MRS into the experimental design and will directly benefit from this improved efficacy.

In vivo model: 2×10⁷ LS174T cells transfected at a MOI of 100 with AdCMVCD were injected into a subcutaneous area in the flank of a nude mouse. Locally, approximately 50 microliters of 3.8 mM 5-FC was injected at the site of the tumor after which these animals were placed in the magnet and evaluated for the presence of 5-FC and the conversion of 5-FC to 5-FU by the adenoviral cytosine deaminase gene. The results demonstrate that the initial time point evaluated was at 10 minutes at which there was a significant peak for 5-FU. The integral of the 5-FU peak exceeded that of the 5-FC peak for 55 minutes.

Correlate the Levels of 5-FU Produced with Therapeutic Outcome in Tumors Treated with Cytosine Deaminase Encoding Adenovirus 5-FC and Radiation Therapy

The enhancement of gene expression in pancreatic and cholangiocarcinoma cell lines was augmented 10-100 times with the Fab-FGF2 redirected virus, and was blocked by an excess of FGF2 and by free heparin sulfate. As shown by Goldman et al., there was enhancement of cellular transduction mediated by Fab-FGF2 redirection of adenoviral infection in human Kaposi's sarcoma (KS) cells. To attempt to distinguish whether the enhanced luciferase gene expression was due to increased gene expression within each cell, or to enhanced transduction efficiency of the AdCMVCD+Fab-FGF2 conjugated virus, redirection experiments with AdCMVLacZ were performed. In vitro, the transduction efficiency of SK-ChA-1 and BXPC-3 cells was substantially improved by the Fab-FGF2 moiety, indicating that significantly greater number of cells were transduced with the redirected virus.

An objective of the present invention was to demonstrate that cytosine deaminase mediated cytotoxicity was enhanced by Fab-FGF2 redirection resulting in greater cytotoxicity. At low viral MOIs in SK-ChA-1 and BXPC-3 cells, there was significantly increased cytosine deaminase gene function measured by conversion of 5-FC into 5-FU, and induction of cytotoxicity. The CFPAC-1 cell line did not demonstrate the degree of enhanced 5-FU production or induction of cytotoxicity with redirected AdCMVCD at low MOIs. Cytotoxicity was only seen with the Fab-FGF2 retargeted virus at high viral doses. This has important implications to treatment of in situ tumors in a clinical setting, as the effectiveness of a single vector administration may be substantially improved. The in vivo experiments indicated that in a multimodality therapy model of human pancreatic cancer, the Fab′-FGF2 redirected AdCMVCD resulted in enhanced tumor growth inhibition compared to native virus alone.

These observations have clear importance in clinical gene therapy applications. Methodologies to enhance the therapeutic effect of the first dose of vector are very important, as many studies have shown decreasing effectiveness of repeat adenoviral vector administration. Additionally, limited clinical experience indicates that multi-modality therapy incorporating neoadjuvant 5-FU chemotherapy and radiation therapy may improve treatment for other refractory malignancies (e.g. rectal or rectosigmoid). Improved treatment of established human tumors is a clinically important goal as patients with both cholangiocarcinoma and pancreatic carcinoma generally present with advanced disease, refractory to current treatment. The present invention indicates an improved tumor response to therapy with AdCMVCD+Fab′-FGF2 compared to AdCMVCD alone in combination with 5-FC treatment and external beam radiotherapy. These results demonstrate that the retargeted AdCMVCD in conjunction with systemic 5-FC administration and external beam radiotherapy was more efficacious in treating established pancreatic tumors in vivo. Thus, this finding validates the efficacy of FGF2-retargeting with this therapeutic gene and a human interventional trial. In conclusion, the enhanced gene delivery obtained in hepatobiliary cancer cells with the Fab-FGF2 redirected adenoviruses translated into enhanced cytotoxicity to pancreatic and cholangiocarcinoma cells utilizing the CD/5-FC toxin gene prodrug system both in vitro and in vivo. These findings provide the rationale for investigating such tropism modified adenoviruses in a clinical setting.

The preliminary data of continuous monitoring conversion of 5-FC to 5-FU via MRS confirm the possibility of detecting the conversion of 5-FC to 5-FU in both an in vitro and in vivo setting. Future studies involves in vivo pancreatic and colon tumor models to evaluate the efficacy of AdCMVCD with and without radiation (5×3 Gy fractions) in the treatment of pancreatic and colon tumor correlated to the conversion of 5-FC to 5-FU detected by MRS. Conversion studies will for the first time allow the use of a noninvasive method to evaluate the role of gene therapy in the treatment of a lethal tumor by determining intratumoral levels of 5-FU as it is converted from 5-FC via the cytosine deaminase gene. Alternate adenoviral, 5-FC and radiation delivery schedules will be devised based on the data obtained from the initial optimization study. These studies will also provide the basis for development of further subcutaneous and liver metastatic models which will allow for the combined treatment of 5-FU and radiation along with noninvasive detection using MRS. The long term goal of these studies is application to the clinical setting for the detection of intratumoral 5-FU established through molecular chemotherapy.

Sensitivity of Glioma Cells to 5-fluorouracil

Systemically administered 5-fluorouracil (5-FU) is the mainstay for chemotherapy of several malignancies, particularly colon, pancreatic and other carcinomas of the gastrointestinal (GI) tract. 5-fluorouracil has also been investigated both in vitro and in vivo for glioma chemotherapy. However, 5-fluorouracil is not routinely used in patients with gliomas, and pharmacokinetic factors, systemic toxicity, and tumor sensitivity have limited its use with other non-gastrointestinal tumors as well. To determine if glioma cells are sensitive to 5-fluorouracil if it is directly administered to the cells, the in vitro toxicity of 5-FU to 4 human glioma cell lines was examined. Two human colon (LS174T, WiDR) and two pancreatic (AsPC-1, BxPC-3) carcinoma cell lines were included as references.

Tumor cells (5×10⁴) were plated in 96 well plates and allowed to adhere overnight. Cells were then incubated continuously in the presence of increasing concentrations of 5-FU (0-200 μg/ml) and assayed for toxicity using an MTS assay (Promega, Madison Wis.) after 5-days. ICₓ values were calculated as previously described. U251MG glioma cells were as sensitive to 5-FU as LS174T and WiDR colon carcinoma cells, while D54MG, U87MG and U118MG glioma cells and AsPC-1 pancreatic carcinoma cells were 3-fold less sensitive (Table 2).

TABLE 2
Sensitivity of human tumor cell lines to 5-FU in vitro

| | | 5-FU (μg/ml) | | |
|---|---|---|---|---|
| Cell Line | Tissue | IC₂₀ | IC₅₀ | IC₈₀ |
| BxPC-3 | Pancreas | 0.01 | 0.04 | 0.69 |
| WiDr | Colon | 0.03 | 0.13 | 1.70 |
| LS174T | Colon | 0.11 | 0.30 | 2.33 |
| U251MG | Glioblastoma | 0.07 | 0.38 | 3.81 |
| U118MG | Glioblastoma | 0.24 | 1.06 | 61.49 |
| U87MG | Glioblastoma | 0.37 | 1.06 | 14.02 |
| D54MG | Glioblastoma | 0.33 | 1.10 | 8.53 |
| AsPC-1 | Pancreas | 0.04 | 1.58 | 39.4 |

Further characterization of the in vitro 5-FU toxicity to human cell lines derived from gliomas (D54MG, U118MG, U251MG, U87MG), prostate (DU145, LNCaP, PC-3), colon (LS174T, WiDR), and pancreatic carcinomas (AsPC-1, BxPC-3, MiaPaca-2) confirmed that that non-gastrointestinal cells are inherently as sensitive to 5-FU as are GI cells (IC₅₀ 0.04-4.97 μg/ml).

Potential Advantages of CD/5-FC Therapy Against Gliomas

The in vitro toxicity of 5-FU to non-gastrointestinal cells suggests that direct delivery of 5-FU to the tumor cells by means of an enzyme/prodrug gene therapy system might be effective for treatment of tumors in vivo. Thus, administration of replication-defective adenovirus (Ad) vectors encoding cytosine deaminase (AdCMVCD) followed by subsequent administration of 5-fluorocytosine (5-FC) may be an effective regimen against glioma cells.

Adenovirus-mediated CD/5-FC therapy offers several distinct advantages over the HSV-tk/GCV systems currently in clinical trials for the treatment of gliomas and other central nervous system tumors. These advantages include a bystander effect that does not require cell-cell contact through gap junctions, which are known to be down-regulated in gliomas. In addition, 5-FU is capable of sensitizing cells to the effects of ionizing radiation. On the other hand, a major factor limiting 5-FU-based therapy and potentially CD/5-FC gene therapy is 5-FU resistance, which is due in part to increased 5-FU catabolism to inactive metabolites by the enzyme dihydropyrimidine dehydrogenase.

Expression of Green Fluorescent Protein in In Vitro Cultured Glioma Cells Following Replication Defective Adenovirus Administration

To determine if administration of genes by replication deficient adenoviral vectors would be effective in glioma cells, delivery of green fluorescent protein to glioma cells via an adenoviral vector was examined. Tumor cells (10⁶) were plated in 6 well plates and allowed to adhere overnight. Cells were then infected with increasing MOI of AdCMVGFP (0-500 pfu/cell) for 1 h at 37° C. Transduction efficiency was assayed by flow cytometry 24 h post infection and quantified as the MOI effecting GFP expression (above background) in x percent of cells (MOIₓ). The results are presented in Table 3.

TABLE 3
Quantification of AdCMVGFP gene transfer to human tumor cells

| | AdCMVGFP (pfu/cell) | | |
|---|---|---|---|
| Cell line | MOI₂₀ | MOI₅₀ | MOI₈₀ |
| U251MG | 1.5 | 3.7 | 7.6 |
| D54MG | 6.1 | 13.0 | 41.4 |
| LS174T | 2.7 | 16.2 | 64.4 |
| WiDr | 6.1 | 26.7 | 84.9 |
| U87MG | 11.0 | 47.9 | 130.8 |
| AsPC-1 | 47.3 | 138.6 | 362.6 |
| U118MG | 36.6 | 139.3 | 367.8 |
| BxPC-3 | 34.9 | 204.7 | 649.0 |

Differential Ad transduction efficiency was not a trivial explanation, since efficiency of reporter gene transfer using an Ad-green fluorescent protein vector (AdCMVGFP) was similar in U251MG, LS174T and WiDR cells (MOI₅₀ 4-27, Table 3).

Administration of CD/5-FC to Cultured Glioma Cells by Replication Deficient Adenovirus.

Next, cytosine deaminase was administered to glioma cells via the replication deficient adenovirus. Glioma cells plated in T25 flasks were infected with AdCMVCD at several multiplicities of infection (MOI 0-300 pfu/cell) for 1 h at 37° C. At twenty-four hours post-infection, cells were harvested, replated at 5×10³ cells/well in 96 well plates, and allowed to adhere overnight. Cells were then incubated continuously in the presence of increasing concentrations of 5-FC (0-200 μg/ml) and IC₅₀ values determined at day 5 as described in Table 2. The results are shown in Tables 4 and 5.

TABLE 4
Sensitivity of human glioma cells to 5-FC upon
infection with AdCMVCD in vitro

| | AdCMVCD MOI | 5-FC IC₅₀ | |
|---|---|---|---|
| Cell Line | (pfu/cell) | μg/ml | r² |

| | | | |
|---|---|---|---|
| D54MG | 10 | — | — |
| | 30 | — | |
| | 100 | 59.9 ± 75.1 | |
| | 300 | 7.9 ± 2.9 | |
| U87MG | 10 | — | — |
| | 30 | — | |
| | 100 | 64.0 | |
| | 300 | 43.0 ± 14.4 | |
| U118MG | 10 | — | — |
| | 30 | — | |
| | 100 | 42.2 | |
| | 300 | 18.7 | |
| U251MG | 10 | 18.4 | 0.96 |
| | 30 | 13.1 ± 11.5 | (p < 0.001) |
| | 100 | 3.3 ± 1.4 | |
| | 300 | 1.0 ± 0.4 | |

TABLE 5
Sensitivity of human gastrointestinal carcinoma cells
to 5-FC upon infection with AdCMVCD in vitro

| | AdCMVCD MOI | 5-FC IC₅₀ | |
|---|---|---|---|
| Cell Line | (pfu/cell) | μg/ml | r² |
| AsPC-1 | 10 | — | 0.14 |
| | 30 | 85.1 ± 9.5 | (p = 0.04) |
| | 100 | 114.5 ± 15.8 | |
| | 300 | 58.6 ± 8.7 | |
| BxPC-3 | 10 | 18.8 ± 5.5 | 0.41 |
| | 30 | 10.2 ± 3.0 | (p < 0.001) |
| | 100 | 2.4 ± 0.5 | |
| | 300 | 1.9 ± 0.4 | |
| LS174T | 10 | 55.3 ± 8.3 | 0.80 |
| | 30 | 8.4 ± 0.9 | (p < 0.001) |
| | 100 | 4.0 ± 0.6 | |
| | 300 | 0.8 ± 0.1 | |
| WiDR | 10 | 33.4 ± 1.9 | 0.96 |
| | 30 | 7.8 ± 0.3 | (p < 0.001) |
| | 100 | 2.7 ± 0.1 | |
| | 300 | 1.4 ± 0.1 | |

U251MG cells were as susceptible as LS174T and WiDR cells to 5-FC after infection with AdCMVCD at 10, 30 and 100 MOI (Tables 4 and 5). As expected, a dose-dependent increase in CD mRNA expression with increasing AdCMVCD MOI was detected in all cell lines tested using a quantitative RT-PCR (TaqMan) assay. The log (IC₅₀) of 5-FC was inversely proportional to the log (AdCMVCD MOI) for most cell lines tested (p<0.01), demonstrating a direct inverse correlation.

AdCMVCD/5-FC produced toxicity results similar to 5-FU and a strong inverse linear relationship between AdCMVCD MOI and 5-FC IC₅₀ (p<0.01) was found with 7 of 8 cell lines tested. These results indicate that the in vitro response of human glioma cells to 5-FU and to AdCMVCD/5-FC is similar to that of human GI tumor cells. In the samples examined, Ad transduction efficiency was highly variable, with gene transfer levels correlating directly with the level of cell surface CAR, but not αv integrin expression. These finding suggest that CAR expression may be a major limiting factor to the success of Ad-based cancer gene therapy, particularly for malignant gliomas. Since only a subset of patients may have tumors that are efficiently infected by Ad vectors upon intralesional injection, screening of patients' tumors for CAR and αv integrin expression may prove extremely useful in selecting patients who might receive maximum benefit from Ad-based gene therapy. Taken together, these results indicate that human glioma cells are not inherently refractory to either 5-FU or to CD/5-FC, suggesting that CD/5-FC-based gene therapy may be of clinical utility for these patients.

In Vivo Administration of CD/5-FC to Glioma Cells

Orthotopic, intracranial murine models using human glioma xenografts may closely approximate the therapeutic response clinically achievable with CD/5-FC enzyme/prodrug therapy. Since the response of human gliomas to CD/5-FC will likely be heterogeneous in the patient population, analysis of AdCMVCD/5-FC-based therapy in multiple models might give a more comprehensive assessment of its potential clinical utility. Three intracranial xenograft models of human glioma in immunodeficient mice are being explored. The models are U87MG, D54MG, and U251 MG.

U87MG and D54MG cells (5×10⁵) were stereotactically injected into the right frontal cortex of SCID mice. Tumors were established for 5 days before intratumoral injection of AdCMVCD (10⁸ or 10⁹ pfu/mouse). Mice were then treated IP with 5-FC (500 mg/kg bid) on days 2-9 post infection and monitored for survival. Kaplan-Meier survival curves and median and 20% survival values were calculated by standard methods. As shown in Tables 6 and 7, intratumoral AdCMVCD plus systemic 5-FC significantly prolonged survival of SCID mice bearing intracranial U87MG or D54MG gliomas (p<0.01), compared to SCID mice treated with an irrelevant Ad vector encoding somatostatin receptor (AdSSTR2). The therapeutic effect was dose-dependent, as a significant increase in median survival was seen with 10⁹ pfu versus 10⁸ pfu of AdCMVD in the U87MG model (Table 8).

TABLE 6
Survival of SCID Mice Bearing Intracranial U87MG
Human Gliomas Treated with Intratumoral AdCMVCD
and Systemic 5-FC

| Treatment | | | Survival | |
|---|---|---|---|---|
| Virus | PFU/Mouse | Drug | Median | 20% |
| Saline | — | 5-FC | 50 | 61 |
| AdCMVCD | 10⁹ | Saline | 51 | 55 |
| AdCMVCD | 10⁹ | 5-FC | 67.5 | 73 |
| AdCMVSSTR | 10⁹ | Saline | 48 | 59 |
| AdCMVSSTR | 10⁹ | 5-FC | 55 | 61 |

TABLE 7
Survival of SCID Mice Bearing Intracranial D54MG
Human Gliomas Treated with Intratumoral
AdCMVCD and Systemic 5-FC

| Treatment | | | Survival | |
|---|---|---|---|---|
| Virus | PFU/Mouse | Drug | Median | 20% |
| Saline | — | 5-FC | 29 | 30 |
| AdCMVCD | 10⁹ | Saline | 33 | 33 |
| AdCMVCD | 10⁹ | 5-FC | 44 | 62 |
| AdCMVSSTR | 10⁹ | Saline | 33 | 37 |
| AdCMVSSTR | 10⁹ | 5-FC | 30 | 34 |

TABLE 8
Survival of SCID Mice Bearing Intracranial
U87MG Human Gliomas Treated with Intratumoral
AdCMVCD and Systemic 5-FC

| Treatment | | | Survival | |
|---|---|---|---|---|
| Virus | PFU/Mouse | Drug | Median | 20% |
| Saline | — | 5-FC | 34 | 34 |
| AdCMVCD | 10⁸ | Saline | 39 | 41 |
| AdCMVCD | 10⁸ | 5-FC | 42.5 | 51 |
| AdCMVCD | 10⁹ | Saline | 34 | 41 |
| AdCMVCD | 10⁹ | 5-FC | 62 | 91 |

These results indicate intratumoral injection of AdCMVCD significantly prolonged the survival of mice bearing established, intracranial D54MG, U251MG, and U87MG tumors following systemic 5-FC administration (p<0.01).

Concurrent Administration of CD/5-FC and Ionizing Radiation to Glioma Cells.

This therapeutic effect of AdCMVCD/5-FC on glioma cells could be further enhanced with concurrent fractionated ionizing external beam radiation (Table 9). U87MG cells (5×10⁵) were stereotactically injected into the right frontal cortex of athymic nude mice. Tumors were established for 5 days before intratumoral injection of AdCMVCD (10⁹ pfu/mouse). Mice were then treated IP with 5-FC (500 mg/kg bid) on days 2-9 post infection, irradiated with local external beam 330 cGy ⁶⁰Co radiation on days 4 and 7 post-infection, and monitored for survival. Kaplan-Meier survival curves and median and 20% survival values were calculated by standard methods. The results are presented in Table 9. Local fractionated external beam radiation (⁶⁰Co) significantly prolonged survival of mice treated with AdCMVCD/5-FC compared to animals receiving AdCMVCD/5-FC alone (p<0.01).

TABLE 9
Survival of Nude Mice Bearing Intracranial U87MG Human
Gliomas Treated with Intratumoral AdCMVCD, Systemic
5-FC, and External Beam Radiation

| Treatment | | | Survival | |
|---|---|---|---|---|
| Virus | Drug | XRT | Median | 20% |
| Saline | 5-FC | — | 28 | 55 |
| Saline | 5-FC | + | 31 | 32 |
| AdCMVCD | 5-FC | — | 31.5 | 67 |
| AdCMVCD | 5-FC | + | 52 | 80 |
| AdCMVLuc | 5-FC | — | 27.5 | 39 |
| AdCMVLuc | 5-FC | + | 56 | 58 |

CD/5-FC with Selectively Replicating Adenovirus

One factor limiting the potential clinical efficacy of Ad-based CD/5-FC therapy is the poor tumor penetration of replication-defective Ad vectors. Intratumoral injection of such vectors is limited to cells adjacent to the needle track. Replication-competent Ad vectors may potentially overcome this limitation by selectively replicating in tumors cells, significantly increasing the level of transgene expression in infected cells as compared to non-replicative Ad while also exerting a direct oncolytic effect. Thus, it was hypothesized that a similar virus capable of selectively replicating only in tumor cells might further enhance the therapeutic response seen with replication-incompetent AdCMVCD/5-FC.

An adenoviral vector (AdE1ACD) was constructed by homologous recombination between the AdE1A-tk adenoviral vector and a linearized plasmid containing the E. coli CD gene into which the adenoviral E1A region had been cloned. This resulted in an adenoviral vector (AdE1ACD) which encodes both CD and a functional E1A gene but lacks the entire E1B region. Deletion of E1B gene permits the selective replication of Ad in cells harboring lesions in the p53 pathway. Replicative virus was generated by infecting 293 cells with 1 MOI of ADE1ACD for two hours. After 48 hours, the cells were harvested. The cells were resuspended at 5×10⁶ cells/ml in media containing 2% heat inactivated fetal bovine serum and were lysed by freezing and thawing or by sonication.

To demonstrate that infection with the selectively replicating virus augments the conversion of 5-FC to 5-FU in infected cells, a “mixing experiment” was performed. LS174T and WiDr human colon cancer cells were infected with AdCD or AdE1ACD for two hours and were harvested after 48 hours of additional incubation. The infected cells were mixed with uninfected cells at ratio of 25% infected cells per total cells. After 2, 4, and 6 days of further incubations, the cells were harvested and assayed for the ability to convert 5-FC to 5-FU. While the conversion of 5-FC to 5-FU decreases with time after mixing with nonreplicative AdCD-infected cells, the conversion of 5-FC to 5-FU remains constant with time after mixing with the AdE1ACD infected cells as a result of increased CD production from newly infected cells.

Various tumor cell types were infected for 48 h with AdE1ACD at MOI 1. The cells were harvested and resuspended at 5×10⁶ cells/ml. Lysates (4× freeze/thaw) were prepared and titered on 293 cells. While no virus could be recovered from cells infected with AdCMVCD, selectively replicating AdE1ACD efficiently replicates to high titer (10⁷-10⁹ pfu/ml) in human GI, prostate and lung tumor cell lines (Table 10). AdE1ACD also expresses functional CD enzyme, as determined by 5-FC to 5-FU conversion assays, and increased CD protein upon intratumoral injection of nude mice bearing subcutaneous LS174T human colon carcinoma xenografts, as determined by CD immunohistochemistry. Thus, intratumoral spread of AdE1ACD may increase CD transgene expression.

TABLE 10
AdE1ACD Replication in Human Tumor Cell Lines

| Cell Line | Tissue | Titer (Log pfu/ml) |
|---|---|---|
| LS174T | Colon | 9.5 |
| WiDR | Colon | 7.6 |
| BxPC-3 | Pancreas | 6.4 |
| AsPC-1 | Pancreas | 7.4 |
| DU145 | Prostate | 6.0 |
| PC-3 | Prostate | 7.5 |

Survival of SCID Mice Bearing Intracranial U87MG Human Gliomas Treated Intratumorally with Selectively Replicating AdE1ACD and Systemic 5-FC.

U87MG cells (5×10⁵), the stringent glioma model, were stereotactically injected into the right frontal cortex of SCID mice. Tumors were established for 5 days before intratumoral injection of AdE1ACD or AdCMVCD (10⁸ or 10⁹ pfu/mouse). Mice were then treated IP with 5-FC (500 mg/kg bid) on days 2-9 post infection and monitored for survival. Kaplan-Meier survival curves and median and 20% survival values were calculated by standard methods. The results are presented in Table 11. Intratumoral administration of 10⁶ pfu of AdE1ACD and systemic 5-FC significantly prolonged the median survival of tumor-bearing animals compared to animals receiving AdE1ACD without systemic 5-FC (p<0.01, Table 11). The therapeutic effect of AdE1ACD/5-FC was critically dependent upon systemic 5-FC with surprisingly minimal anti-tumor response directly attributable to viral oncolysis.

TABLE 11
Survival of SCID Mice Bearing Intracranial U87MG Human
Gliomas Treated Intratumorally with Conditionally-
replicative AdEIACD and Systemic 5-FC

| Treatment | | | Survival | |
|---|---|---|---|---|
| Virus | PFU/Mouse | Drug | Median | 20% |
| Saline | — | 5-FC | 57 | 60 |
| AdE1ACD | 10⁶ | Saline | 49 | 54 |
| AdE1ACD | 10⁶ | 5-FC | 84 | 86 |

Additional experiments were performed on intracranial tumors derived from all three glioma models. SCID mice bearing intracranial U251MG gliomas that were treated with AdE1ACD/5-FC had a significant survival advantage (median 39 days) over animals receiving replication-defective AdCMVCD/5-FC (36 days), AdE1ACD/saline (32 days), or no virus/5-FC (23 days, p<0.01; Table 12). Similar results were obtained with SCID mice bearing intracranial U87MG gliomas (AdE1ACD/5-FC 84 days, AdE1ACD/saline 49 days, no virus/5-FC 57 days, p<0.01; Table II and Table 12). Taken together, these results demonstrate the superior efficacy of the selectively replication-competent AdE1ACD for CD/5-FC therapy.

TABLE 12
Intratumoral Ad-mediated CD/systemic 5-FC therapy in three
intracranial SCID mouse models of human glioma

| | AdCMVCD/5-FC^{a} | | AdE1ACD | AdE1ACD |
|---|---|---|---|---|
| Tumor | Expt 1 | Expt 2 | Saline^{b} | 5-FC^{b} |
| U87MG | 1.13 | 1.35 | 0.86 | 1.47 |
| D54MG | 1.52 | 2.47 | ND | ND |
| U251MG | 1.57 | ND | 1.39 | 1.70 |

^{a}Relative increase in median survival of animals receiving AdCMVCD (10⁹ pfu) and systemic 5-FC (500 mg/kg bid ip for 7 days) versus animals receiving no virus plus 5-FC. Animals receiving AdCMVCD/saline displayed no survival advantage (data not shown).
^{b}Relative increase in median survival of mice receiving AdE1ACD (10⁷ pfu) and either saline or systemic 5-FC (500 mg/kg bid ip for 7 days) versus animals receiving no virus plus 5-FC. While the therapeutic response of these tumors was variable (U251MG > D54MG > U87MG), the response correlated well with the in vitro sensitivity of these cells to 5-FU and to AdCMVCD/5-FC.

Replication Defective Adenovirus Expressing a Cytosine Deaminase (CD)/Uracil Phosphoribosyltransferase (UPRT) Fusion Protein.

Another factor that may limit CD/5-FC efficacy is the intratumoral expression of dihydropyrimidine dehydrogenase (DPD), the rate-limiting enzyme in 5-FU catabolism. DPD activity in peripheral blood mononuclear cells and hepatocytes significantly affects 5-FU pharmacokinetics after systemic administration, catabolizing over 90% of the injected dose to inactive metabolites. The remaining 10% of 5-FU is the active fraction that reaches GI tumors, which express low levels of DPD. However, cell lines derived from non-GI tumors (gliomas) express high levels of DPD mRNA. To overcome this potential limitation, a replication-defective Ad vector was constructed encoding a fusion protein between CD and an additional enzyme, uracil phosphoribosyltransferase (UPRT, AdCDUPRT). UPRT catalyzes the first step in 5-FU anabolism, the production of 5-fluoruridine monophosphate (5-FUMP). It was hypothesized that simultaneous expression of CD and UPRT may overcome intratumoral DPD expression by shunting CD-produced 5-FU away from the DPD-dependent catabolic pathway and into the UPRT-mediated anabolic pathway.

To test this hypothesis, 5-FC toxicity (IC₅₀) was compared in cells infected with AdCMVCD versus AdCDUPRT. Results with human DPD-positive glioma cells, and with prostate cancer cells, demonstrated an 18-280 fold decrease in 5-FC IC₅₀ with AdCDUPRT-infected versus AdCMVCD-infected cells at an equal multiplicity of infection (MOI 100 pfu/cell; Table 13). Potentiation of 5-FC toxicity by CDUPRT was due to UPRT, since expression of CDUPRT, but not CD, could increase the toxicity of glioma cells to 5-FU (571-1125 fold decrease in 5-FU IC₅₀ at MOI of 100 pfu/cell; Table 14). These results demonstrate the potential of UPRT to increase CD/5-FC toxicity in vitro. Taken together, these results show that selective sensitization of tumors by direct intratumoral gene transfer of a prodrug-activating enzyme may hold promise as a means to improve the therapeutic index of standard chemotherapeutic drugs. In Table 13 mean 5-FC IC₅₀ was obtained from 2-5 separate experiments. Cells were infected with AdCMVCD or AdCDUPRT at the indicated MOI (pfu/cell) for 1 h at 37° C. 5-FC toxicity was determined by MTS assay 5 days after drug addition.

TABLE 13
AdCMVCD and AdCDUPRT 5-FC toxicity

| | | | 5-FC IC₅₀ (μg/ml) | | |
|---|---|---|---|---|---|
| Cell Line | Tissue | MO | AdCMVCI | AdCDUPRT | Enhancement |
| D54MG | Glioma | 10 | 37.8 | 0.31 | 122 |
| | | 30 | 19.6 | 0.17 | 115 |
| | | 100 | 9.1 | 0.06 | 152 |
| | | 300 | 4.7 | 0.02 | 235 |
| U251MG | Glioma | 10 | 9.9 | 0.04 | 248 |
| | | 30 | 9.1 | ND | |
| | | 100 | 2.8 | 0.01 | 280 |
| | | 300 | ND | ND | |
| U87MG | Glioma | 10 | >200 | 17.71 | |
| | | 30 | >200 | 0.88 | |
| | | 100 | 95.9 | 1.18 | 81 |
| | | 300 | 33.0 | 0.92 | 36 |
| DU145 | Prostate | 10 | >200 | 21.38 | |
| | | 30 | ND | 13.00 | |
| | | 100 | 25.0 | 0.62 | 42 |
| | | 300 | ND | 0.28 | |
| LNCaP | Prostate | 10 | 4.6 | 0.021 | 219 |
| | | 30 | ND | ND | |
| | | 100 | 1.0 | 0.007 | 143 |
| | | 300 | ND | 0.0001 | |
| PC-3 | Prostate | 10 | 26.6 | 1.26 | 21 |
| | | 30 | ND | 1.54 | |
| | | 100 | 4.2 | 0.23 | 18 |
| | | 300 | 4.6 | 0.07 | 67 |

(ND, not determined)

TABLE 14
AdCDUPRT Enhancement of 5-FU toxicity

| | | 5-FU IC₅₀ (μg/ml) | | |
|---|---|---|---|---|
| Cell Line | MOI | AdCMVCD | AdCDUPRT | Enhancement |
| D54MG | 0 | 1.3 | | |
| | 10 | | 0.0023 | |
| | 30 | | 0.0016 | |
| | 100 | 0.9 | 0.0010 | 900 |
| | 300 | | 0.0006 | |
| U251MG | 0 | 0.4 | | |
| | 10 | | 0.0016 | |
| | 30 | | 0.0015 | |
| | 100 | 0.4 | 0.0007 | 571 |
| | 300 | | 0.0015 | |
| U87MG | 0 | 1.8 | | |
| | 10 | | 0.0081 | |
| | 30 | | 0.0025 | |
| | 100 | 2.7 | 0.0024 | 1125 |
| | 300 | | 0.0007 | |

Cells infected with AdCMVCD or AdCDUPRT at the indicated MOI (pfu/cell) for 1 h at 37° C. 5-FC toxicity (IC₅₀) determined by MTS assay 5 days after drug addition.

In Vitro Quantification of Ad Gene Transfer Efficiency and CAR and αv Integrin Expression

Indirect immunocytofluorimetry assays are developed for quantification of Ad gene transfer efficiency in vitro and for analysis of CAR and αv integrin expression in cultured primary pediatric brain tumors. Short-term primary cultures of pediatric brain tumors are established from surgically excised tumor specimens obtained from patients at University of Alabama Hospital. Ad gene transfer efficiency is quantified as described and statistical comparisons of the MOI necessary to achieve 50% transfection (MOI₅₀) are made. Cell surface expression of CAR, αvβ3, and αvβ5 integrin proteins are determined by indirect immunofluorescence using RmcB (ATCC), LM609 (Chemicon, Temecula Calif.) and P1F6 (Chemicon), respectively. U118MG (CAR−) and human CAR (hCAR)-transfected U118MG (CAR+) cells serve as controls.

CAR Staining of Frozen and Paraffin-Embedded Tissue Sections

CAR expression in pediatric gliomas likely dictates the clinical success of Ad-based gene therapy. A facile immunohistochemical method to determine CAR expression on histological samples is devised. Various methods are explored to detect CAR on both fresh-frozen and fixed, paraffin embedded sections of freshly excised tumors using the RmcB monoclonal antibody. RmcB binds specifically to CAR by immunoblot, immunoprecipitation and flow cytometry assays, but no data exists on either its use in immunohistochemistry or on the effects of tissue processing on the RmcB epitope of CAR. These effects are explored by processing the following cell lines into standard paraffin-embedded histological sections using HistoGel (Lab Storage Systems, Warrenton, Mo.): CHO and a CHO clone stably expressing human CAR (CHO-hCAR, AQ17) and U118MG and U118MG-hCAR, which has already been constructed and shown to express high levels of CAR by RmcB flow cytometry. Various fixatives are tested prior to HistoGel embedding and paraffin processing of these cells to optimize RmcB staining specificity and intensity. Antigen retrieval techniques, including low and high temperature microwave processing, are explored if needed. Pediatric tumor samples are screened for CAR expression as well as expression of αvβ3 and αvβ5 integrins using either heterodimer specific antibodies (eg. LM609 and P1F6) or subunit specific antibodies (eg. αv-P3G8, β3-AB1932, β5-AB1926, Chemicon) as previously described. These results are useful in determining: (1) the prevalence of CAR/αv integrin expression on pediatric gliomas; (2) heterogeneity of CAR/αv integrin expression on single tumor samples; and, (3) the pattern of CAR/αv integrin expression on CNS cell types, as well as in correlating CAR/αv integrin expression with Ad gene transfer efficiency.

CD/5-FC-Based Enzyme/Prodrug Therapy in Animal Models of Pediatric Glioma.

Two animal model systems are used to assess the efficacy of the CD/5-FC-based gene therapies. SCID mice are employed for studies with orthotopic, intracranial xenografts of human gliomas. These animals are monitored for survival following therapy and moribund mice are sacrificed and their brains harvested for pathological examination with routine hematoxylin/eosin staining. Due to lethality of local external beam radiation to the cranium of SCID mice, Balb/c nude mice are used for studies with concurrent radiation. Appropriate groups are included to control for any intermodel differences in survival following identical treatment protocols. Subcutaneous tumor models using athymic nude mice are utilized to monitor the kinetics of tumor volume reduction and the potential for and latency of rebound tumor growth. Results of these tumors are compared to the response of subcutaneous WiDR human colon xenografts, and potentially other GI tumors such as BxPC-3 pancreatic carcinomas, to assess the differential in response of tumors from these tissues. Immunohistochemical staining for CD and Ad hexon expression on both intracranial and subcutaneous tumors is performed to assess CD and Ad gene expression efficiency, distribution, and kinetics after intratumoral AdCMVCD injection.

Animal Studies Investigating Intratumoral AdCMVCD with Systemic 5-FC Therapy.

Further studies with AdCMVCD/5-FC and concurrent external beam radiation are being performed with all three cell lines in the intracranial and subcutaneous nude mouse models. Studies with concurrent CD/5-FC and radiation are limited to the use of replication-incompetent AdCMVCD virus. The results with AdE1ACD in intracranial U87MG SCID mouse xenografts has prompted further exploration of the efficacy of this therapy in all three glioma models. In addition to allowing assessment of differential efficacy of this approach, results with D54MG and U87MG tumors permit evaluation of transduction efficiency on efficacy of AdE1ACD therapy, since these cell lines displayed similar 5-FU and AdCMVCD/5-FC sensitivities in vitro. To determine the extent of viral oncolysis versus 5-FU mediated cytotoxicity, an analogous replication-competent vector encoding HSV-tk is utilized (AdE1Atk).

Mechanism of Interaction of CD-5-FC-Based Gene Therapy Combined with Conventional Treatment

Clonogenic survival assays are performed in vitro, with fractionated radiation therapy as described above. These data provide the foundation for in vivo studies with these glioma lines. A series of in vitro studies is conducted with eniluracil, the inhibitor of Dihydropyrimidine dehydrogenase enzyme. DPD expression has been shown to be low in colon cell lines, and high in gliomas by microarray analysis. By inhibiting DPD, an even greater 5-FU effect is achieved. DPD expression is quantified by TaqMan and DPD enzyme assays on cell lines (colon/pancreatic carcinomas and gliomas) as previously described. Importantly, a large panel of pediatric brain tumor tissues collected over the last 13 years is screened and cryopreserved in a repository. This provides clinical relevance to this inhibitory approach. The effect is assessed by in vitro toxicity of 5-FU±eniluracil with gliomas and GI lines as described above. This is followed by extensive dose-response testing by in vitro toxicity of AdCMVCD+5FC±eniluracil with gliomas and GI lines. These data provide a rational basis to conduct defined in vivo studies using both U87MG and U251MG intracranial models with the most efficacious combinations of AdCMVCD+5FC, with or without various doses of eniluracil. These studies are preceded by in vivo testing of eniluracil pharmacokinetics/pharmacodynamics to assess the most appropriate route and dosing parameters to achieve an effective biological effect in intracranial brain tumors.

Tumor Cells, Animals, Chemicals, Antibodies and Recombinant Adenoviruses

Human glioma cell lines D54MG and U251MG (from Dr. Darell D. Bigner, Duke University, Durham, N.C.) and U87MG (American Type Culture Collection, Manassas, Va.), and HEK293 human embryonic kidney cells (Microbix Biosystems, Ontario, Canada) were cultured in DMEM/F12 (Mediatech, Herndon, Va.) containing 10% fetal bovine serum (FBS) (Summit Biotechnology, Fort Collins, Colo.). All cells were cultured at 37° C. in a 5% CO₂ atmosphere without antibiotics.

Human pancreatic cancer cells S2-013 and S2-VP 10 (from Dr. Anthony Hollingsworth, University of Nebraska, Lincoln, Nebr.), MIA PaCa-2, BxPC-3 and PANC-1 (American Type Culture Collection, Manassas, Va.), and HEK293 human embryonic kidney cells (Microbix Biosystems Inc., Ontario, Canada) were cultured in DMEM/F12 (Mediatech, Herndon, Va.) containing 10% fetal bovine serum (FBS) (Summit Biotechnology, Fort Collins, Colo.). Panc2.03 human pancreatic cancer cells (from Dr. Elizabeth Jaffee, The Johns Hopkins University, Baltimore, Md.) were cultured in RPMI-1640 containing 1.0 mM sodium pyruvate, 0.1 mM non-essential amino acids, 0.01 mg/ml bovine insulin (Mediatech) and 10% FBS (Summit Biotechnology). All cells were cultured at 37° C. in a 5% CO₂ atmosphere without antibiotics. Female nude athymic mice were purchased from the Frederick Cancer Research Facility (Bethesda, Md.) and housed under aseptic conditions in microisolator cages and experiments were carried out according to the Institutional Animal Care and Use Committee approved protocols. 5-FC and 5-FU were purchased from Sigma-Aldrich (St. Louis, Mo.) and SP Pharmaceuticals (Albuquerque, N. Mex.), respectively.

A replication-deficient E1- and E3-deleted AdbCDwt recombinant Ad vector encoding the wild-type CD enzyme from Escherichia coli (CD; EC 3.5.4.1) gene (codA) under control of the human CMV immediate early promoter, was constructed by two plasmid rescue in HEK293 cells using pACCMVpLpA shuttle vector and pJM17 rescue vector as described previously. A replication-deficient E1-and E3-deleted AdbCD-D314A recombinant adenoviral vector encoding the mutant codA gene driven by the CMV promoter was developed using pAdEasy system (Quantum Biotechnologies, Montreal, Canada) as per the manufacturer's protocol. Briefly, to generate pShuttle-bCD-D314 plasmid, the fragment containing bCD-D314A was removed from pETHT:bCD. plasmid by restriction digestion, blunted and cloned into a pShuttle-CMV plasmid (Quantum Biotechnologies). The insert sequence and orientation were confirmed by restriction enzyme mapping and partial sequencing analysis. The resultant plasmid was linearized and cotransfected with pAdEasy-1 plasmid (Quantum Biotechnologies) into Escherichia coli BJ5183 bacteria. Recombinant clones were confirmed by polymerase chain reaction analysis, linearized and transfected into permissive HEK293 cells using the Effectene lipid-based transfection method (QIAGEN, Chatsworth, Calif.) to generate AdbCD-D314A recombinant adenovirus which was isolated from a single positive plaque and passed through three rounds of plaque purification and subsequently confirmed by PCR. Viruses were propagated on HEK293 cells and purified twice by centrifugation on CsCl gradients. The quantity of viral particles was monitored by absorbance of the dissociated virus at A₂₆₀ nm. Viral titer was measured by a 50% tissue culture infectious dose (TCID₅₀) assay. Briefly, HEK293 cells were plated into 96 well plates at 5×10³ cells/well, and allowed to adhere overnight. Next day, serial dilutions of the viral stock were added directly to cells. Cells were incubated for 10 days, and cytopathic effect was determined using a crystal violet staining assay. Cell culture medium was removed and surviving cells were then fixed and stained with 2% (w/v) crystal violet (Sigma-Aldrich) in 70% ethanol for 3 h at room temperature. The plates were washed extensively, air-dried and the ratio of positive wells with observable cytopathic effect for each viral preparation was determined. The viral titer was calculated by the Karber equation: T=10^{1+D(S-0.5)}/V, where T is infectious titer in TCID₅₀/ml, D is the log₁₀ of the dilution, S is the log₁₀ for the initial dilution plus the sum of ratios, and V is the volume in ml of the diluted virus used for infection. Multiplicity of infection (MOI) for subsequent experiments was expressed as TCID₅₀ per cell.

In Vitro Cytotoxicity Assay Using Crystal Violet Staining

To measure the cytotoxicity of 5-FU and 5-FC, glioma cells or pancreatic cancer cells were plated into 96-well tissue culture plates at 5×10³ cells/well, and allowed to adhere overnight. Next day, serial dilutions of 5-FU or 5-FC were added directly to cells. Cells were incubated for 5 days, and relative cell density was determined using a crystal violet staining assay. Cell culture medium was removed and surviving cells were then fixed and stained with 2% (w/v) crystal violet (Sigma-Aldrich) in 70% ethanol for 3 h at room temperature. The plates were washed extensively, air-dried and optical density was measured at 570 nm using a V Max plate reader (Molecular Devices Corporation, Sunnyvale Calif.). Fractional cell survival at each drug concentration was calculated as the ratio of absorbance at 570 nm of cells incubated in the presence versus absence of drug, corrected for background absorbance of media alone. Fractional cell survival data were plotted against drug concentration and IC₅₀ values extrapolated by piecewise linear regression as the concentration of drug producing a 50% reduction in corrected absorbance.

For AdbCD-D314A/5-FC and AdbCDwt/5-FC cytotoxicity experiments, the target cells were plated into 96-well tissue culture plates at 5×10³ cells/well, and allowed to adhere overnight. Twenty-four hours later cells were infected with AdbCD-D314A or AdbCDwt at various MOI. Next day fresh media supplemented with serial dilutions of 5-FC were added, and 24 h after prodrug treatment, glioma cells were either mock-irradiated or irradiated at 89 cGy/min using a ⁶⁰Co gamma irradiator (Picker Unit, Cleveland, Ohio) and 5-FC cytotoxicity (IC₅₀) was determined at 5 days using a crystal violet staining assay.

CD Conversion Assay In Vitro

Human glioma cells or pancreatic cancer cells were seeded into 25 cm² flasks, 24 h prior to infection. Cells were exposed to AdbCD-D314A and AdbCDwt at various MOI for 2 h, culture media was removed and fresh media was added. Forty-eight hours later, the cells were trypsinized and resuspended in CD lysis buffer (100 mM Tris, 1 mM EDTA, 1 mM DTT, pH 7.8). Cells were frozen and thawed three times, and after centrifugation the supernatant assayed for protein concentration (Bio-Rad Laboratories, Hercules, Calif.). To start each reaction, ³H-5-FC was added. After various time points, the reactions were stopped with the addition of 95% ethanol. 5-FC/5-FU (Pharmacia, Peapack, N.J.) standards were added to each tube and the whole sample spotted on flexible plates for thin layer chromatography (Whatman, Clifton, N.J.). These were run in 86:14 butanol:water to separate the ³H-5-FU (top) from the ³H-5-FC (bottom) spots and all were cut out and put into scintillation fluid, and counted using a 1900 TR Liquid Scintillation Analyzer (Packard Instrument Co., Downers Grove, Ill.). To determine the specific activity of samples, ³H-5-FU formed (pmol) was plotted against time. The slope of the graph (pmol formed per min) was then divided by the amount of protein added to obtain the CD activity (expressed as pmol/min/mg protein).

Clonogenic Survival Assay

At 24 h after infection with 50 MOI AdbCDwt or AdbCD-D314A, cells were trypsinized and diluted to an appropriate cell density and placed into 6-well culture plates and allowed to adhere overnight. Next day, 5-FC were added directly to cells at 4 μg/ml, and after 24 h incubation at 37° C. cells were either mock-irradiated or irradiated at 2 Gy using a ⁶⁰Co gamma irradiator (Picker Unit) and were then returned to the incubator and cultured additionally for 21 days. Cells were then fixed with 70% ethanol and stained with 2% (w/v) crystal violet (Sigma-Aldrich). Colonies comprising at least 50 cells were counted. The plating efficiencies were calculated as the number of colonies divided by the number of test cells plated for each data point. Plating efficiencies were referenced back to the mock-irradiated control plating efficiency to determine the surviving fraction for each dose.

CD Conversion in Tumor Xenografts

For in vivo CD conversion assay, 2×10⁷ D54MG human glioma cells were injected s.c. into female athymic nude mice. When tumors reached 6-8 mm in diameter, groups of 3 tumors were injected with 1.5×10⁸ TCID₅₀ of AdbCDwt or AdbCD-D314A, and mock-irradiated or irradiated at 2 Gy one day before or after injection using a ⁶⁰Co gamma irradiator. Tumors were dissected at different times after Ad injection, and one-third of the tumor was crushed with a mortar and pestle in 100 μl Complete Mini protease inhibitor buffer (Roche Diagnostics, Indianapolis, Ind.). Tumor tissues were frozen and thawed three times, and after centrifugation the supernatant fluids assayed for protein concentration and tested for CD conversion as described above.

Subcutaneous Human Glioma Xenograft Model

To assess anti-tumor effects on established solid tumors, D54MG glioma cells (2×10⁷) were injected s.c. into female athymic nude mice. Treatment was started 14 days post-tumor cell injection at the time of established tumor growth (tumors were 6-8 mm in diameter), noted as Day 0. Animals were randomly divided into groups receiving different treatments: AdbCDwt plus 5-FC; AdbCD-D314A plus 5-FC; AdbCDwt and 5-FC plus radiation; AdbCD-D314A and 5-FC plus radiation; 5-FC plus radiation; and 5-FC alone as control. Mice were injected i.t. with 1×10⁸ TCID₅₀ per tumor AdbCDwt or AdbCD-D314A on Days 0, 7, and 14. 5-FC was administered i.p. at 500 mg/kg twice daily 5 days/week for 3 weeks. Radiation treatment was 2 Gy from a ⁶⁰Co gamma irradiator on days 4, 7 and 10. For the next experiment, animals were injected 17 days post-D54MG tumor cell injection (Day 0) i.t. with 1×10⁸ TCID₅₀ AdbCD-D314A or Days 0, 7, and 14 and irradiated with 5 Gy fractions on Days 4, 7, and 10 (15 Gy total) on Days 0, 7, 10, 14, 17 and 21 (30 Gy total). Two groups of mice received 5-FC i.p. at 500 mg/kg twice daily 5 days per week for 3 weeks beginning on Day 0. Tumor size was monitored twice a week with Vernier calipers. Tumor surface area (length×width in mm²) was calculated for each group of 10 mice and plotted as a percentage change over time relative to the mean size on Day 0 for each group.

Intracranial Survival Study

D54MG tumors were established intracranially in athymic nude mice as described. Briefly, mice were anesthetized by i.p. administration of ketamine (20 mg/ml) and xylazine (0.3 mg/ml) in saline at 0.07 ml/10 g of body weight. A midline scalp incision was made, and a 0.5-mm burr hole was drilled 1.5-2.0 mm to the right of midline and 1 mm anterior to the coronal suture. D54MG cells were injected stereotactically at 0.5×10⁶ cells (1×10⁸/ml), using a 250 μl Hamilton syringe with a prepared 30 gauge needle mounted in a Stoelting stereotaxic apparatus. A plastic sleeve surrounding the needle allowed reproducible injections of tumor cells, saline or Ad to a depth of 2.5 mm. The needles were left in place for 2 min to minimize reflux of the tumor cells along the needle track. The scalp wounds were closed with Tissu-Mend glue to avoid scatter dose from metallic wound clips during radiation treatment. The mice were placed on a heating pad in sterile microisolator polycarbonate cages and allowed to awaken from anesthesia. Tumors were allowed to grow for 6 days before the start of treatment (Day 0). Animals were randomly divided into groups receiving different treatments: 3.2×10⁷ TCID₅₀ AdbCD-D314A in 10 μl saline; AdbCD-D314A in combination with radiation; AdbCD-D314A plus 5-FC; AdbCD-D314A plus 5-FC in combination with radiation, intratumoral saline followed by intraperitoneal 5-FU and intratumoral saline plus 5-FC and radiation. On the day after AdbCD-D314A injection, mice were treated i.p. with either saline or 5-FC twice daily at 500 mg/kg on Days 0 to 4, and 7 to 11 and monitored daily for survival. Radiation therapy consisted of 15 Gy ⁶⁰Co delivered in 5 Gy fractions to the whole brain, on Days 1, 3 and 7. Irradiated mice were immobilized in individual Lucite chambers with their heads positioned in an 8×32 cm collimated field. Animals were shielded with 3 cm of lead placed over the chamber, extending from behind the ears to the tail. When tumor bearing mice displayed overt signs of neurological dysfunction, manifested primarily as a hunched appearance, lack of grooming, and lack of avoidance behavior when handled, they were killed by lethal CO₂ inhalation, and their brains were harvested for histopathological examination, confirming the presence of progressive tumor in all dead mice.

All error terms are expressed as the standard deviation of the mean. Significance levels for comparison of differences between groups in the in vitro experiments were analyzed by Student's t test. In the animal model tumor therapy studies, the treatment groups were compared with respect to tumor size and percent of original tumor size over time. To test for significant differences in tumor size between treatment groups, one-way analysis of variance test was conducted. Kaplan-Meier survival curves were analyzed by the log-rank test, and pairwise multiple comparisons were made using Holm-Sidak method.

To determine the sensitivity of glioma cell lines to 5-FU, cells were treated with increasing concentrations of 5-FU, and the cytotoxicity of this drug was determined by measuring surviving cells using the crystal violet staining method. The susceptibility to cytotoxic effects of 5-FU was variable in different glioma cell lines. The concentration of 5-FU to produce 50% viable cells (IC₅₀, 50% inhibitory concentration) was 3.9±0.8 μg/ml for D54MG cells, 2.5±1.2 μg/ml for U251MG cells, and 0.9±0.5 μg/ml for U87MG cells.

Enzyme Activity of bCDwt and bCD-D314A In Vitro

The replication-defective recombinant Ad vectors AdbCDwt and AdbCD-D314A, encoding wild type codA and mutant codA (harboring substitution of an alanine for the aspartic acid at position 314 in the CD protein) genes, respectively, were constructed under control of the CMV promoter. In order to determine the CD conversion activity, human glioma cells were infected with AdbCD-D314A or AdbCDwt and the enzyme activity was determined by measuring the conversion of ³H-5-FC to ³H-5-FU. The results of CD conversion assays demonstrated that the enzyme activity was elevated 183, 206 and 101-fold for D54MG, U87MG and U251 MG cells infected with 2 MOI of AdbCD-D314A compared to 2 MOI AdbCDwt, respectively. Enzyme activity was enhanced 48, 69 and 12-fold for D54MG, U87MG and U251MG cells infected with 50 MOI of AdCD-D314A respectively, in comparison with the levels of enzyme activity in AdbCDwt infected cells (Table 15). In addition, ionizing radiation at 2 Gy produced enhanced CD conversion of 1.6 and 1,8-fold for D54MG cells infected with 20 MOI of AdbCD-D314A and AdbCDwt, respectively, in comparison with the level of enzyme activity in mock-irradiated cells.

TABLE 15
CD conversion activity of AdbCDwt and
AdbCD-D314A in human glioma cells in vitro

| | CD conversion (pmol/min/mg)^{a} | | | |
|---|---|---|---|---|
| | AdbCDwt | | AdbCD-D314A | |
| | 2 MOI | 50 MOI | 2 MOI | 50 MOI |
| D54MG | 4.6 ± 0.1 | 58.5 ± 37.8 | 842.6 ± 186.6* | 2763.6 ± 120.5** |
| U87MG | 1.8 ± 0.7 | 31.0 ± 13.9 | 370.1 ± 22.9* | 2133.8 ± 160.7** |
| U251MG | 8.4 ± 0.2 | 94.8 ± 60.2 | 848.6 ± 182.8* | 1161.6 ± 316.7*** |

^{a}CD activity was determined by measuring the conversion of ³H-5-FC to ³H-5-FU;
*p < 0.001 for 2 AdbCD-D314A compared to 2 MOI AdbCDwt treated cells;
**p < 0.005 for 50 MOI AdbCD-D314 comparison with 50 MOI AdbCDwt;
***p < 0.05 for 50 MOI AdbCD-D314A in comparison with 50 AdbCDwt.

[symbol] TABLE 16
CD conversion activity of AdbCD-D314A and AdbCDwt alone
and in combination with ionizing radiation in D54MG
glioma cells in vitro

| | CD conversion (pmol/min/mg)^{a} | |
|---|---|---|
| | 20 MOI^{b} | 20 MOI + 2 Gy |
| AdbCD-D314A | 2358.4 ± 227.6* | 3644.8 ± 258.8**,*** |
| AdbCDwt | 28.3 ± 10 | 51.1 ± 13.9 |

^{a}CD activity was determined by measuring the conversion of ³H-5-FC to ³H-5-FU;
^{b}Cells were mock irradiated or irradiated with 2 Gy using a ⁶⁰Co gamma irradiator one day before AdbCD-D314A or AdbCDwt infection;
*p = 0.0048 for AdbCD-D314A in comparison with AdbCDwt;
**p = 0.034 for AdbCD-D314A plus irradiation compared to AdbCD-D314A alone;
***p = 0.0026 for AdbCD-D314A plus irradiation in comparison with AdbCDwt plus irradiation.

AdbCDwt/5-FC and AdbCD-D314A/5-FC Sensitivity

To determine the 5-FC sensitivity of tumor cells to Ad-mediated suicide gene expression, human glioma cells were infected with 10 MOI AdbCDwt or AdbCD-D314A and treated with increasing concentrations of 5-FC, and the relative cell viability was determined using the crystal violet staining assay. IC₅₀ values for glioma cells are shown in Table 17. Expression of the mutant bCD protein in glioma cells significantly increased their sensitivity to 5-FC treatment. The IC₅₀ with 5-FC administration decreased by 7,8-fold for D54MG, 32.9-fold for U87MG, and 8,1-fold for U251MG in AdbCD-D341A-infected cells in comparison with AdbCDwt-infected cells. The viability of AdbCD-D314A-infected cells incubated with 5-FC decreased in a MOI-dependent manner.

TABLE 17
Cytotoxicity of AdbCDwt/5-FC and AdbCD-D314A/5-FC ag:
human glioma cells in vitro

| | 5-PC IC₅₀ (μg/ml) | |
|---|---|---|
| | AdbCDwt | AdbCD-D314A |
| D54MG | 368.2 ± 52.4 | 47.4 ± 9.8* |
| U87MG | 121.6 ± 19.3 | 3.7 ± 1.1** |
| U251MG | 180.1 ± 24.5 | 22.3 ± 1.3* |

*p < 0.05 for AdbCD-D314A in comparison with AdbCDwt;
**p < 0.005 for AdbCD-D314A in comparison with AdbCDwt.

Combined Treatment with AdbCD-D314A/5-FC and Ionizing Radiation Enhanced Glioma Cytotoxicity

To investigate whether combined treatment with AdbCD-D314A/5-FC and radiation treatment produces increased cytotoxicity of cancer cells in vitro, D54MG glioma cells were first infected with AdbCD-D314A or AdbCDwt at 10 MOI. As shown in Table 18, the combination of AdbCD-D314A infection and 2 Gy radiation treatment one day later produced significant enhanced cell death in comparison with AdbCD-D314A and 5-FC treatment (p<0.05). In contrast, the comparisons of IC₅₀ values for groups treated with AdbCD-D314A/5-FC one day after radiation treatment or on the same day versus the AdbCD-D314A/5-FC-treated D54MG cells showed no significant differences between the groups (p>0.05). A similar increase in cell killing following combined treatment with AdbCD-D314A/5-FC and ionizing radiation one day after infection was obtained with U87MG and U251MG cells. In each instance, the 5-FC IC₅₀ value was substantially lower in AdbCD-D314A as compared to AdbCDwt-infected cells.

TABLE 18
Cytotoxicity of combination treatment with radiation and
suicide gene therapy against human glioma cells

| | 5-FC IC₅₀ (μg/ml)^{a} | | | |
|---|---|---|---|---|
| | Ad | IR + Ad | Ad/IR | Ad + IR |
| AdbCD-D314A | 52.7 ± 9.1* | 40.2 ± 8.2 | 45.1 ± 5.1 | 27.3 ± 5.3** |
| AdbCDwt | 351.0 ± 38.4 | ND | ND | 247.3 ± 42.4** |

^{a}D54MG glioma cells were infected with 10 MOI of AdbCD-D314A or AdbCDwt, and mock-irradiated (Ad) or irradiated with 2 Gy using a ⁶⁰Co gamma irradiator one day before (IR + Ad), concurrently (Ad/IR) or one day after Ad infection (Ad + IR). 5-FC cytotoxicity was determined on day 5 after Ad infection by crystal violet staining assay and cell survival was corrected for background absorbance of IR alone control;
*p < 0.005 for AdbCD-D314A in comparison with AdbCDwt;
**p < 0.05 for AdbCD-D314A followed by irradiation or AdbCDwt followed by irradiation compared to AdbCD-D314A or AdbCDwt alone, respectively.

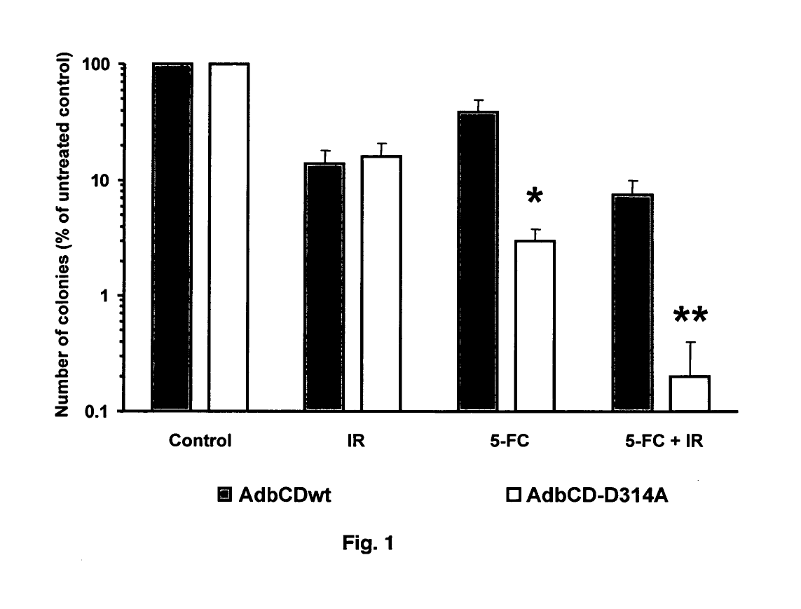

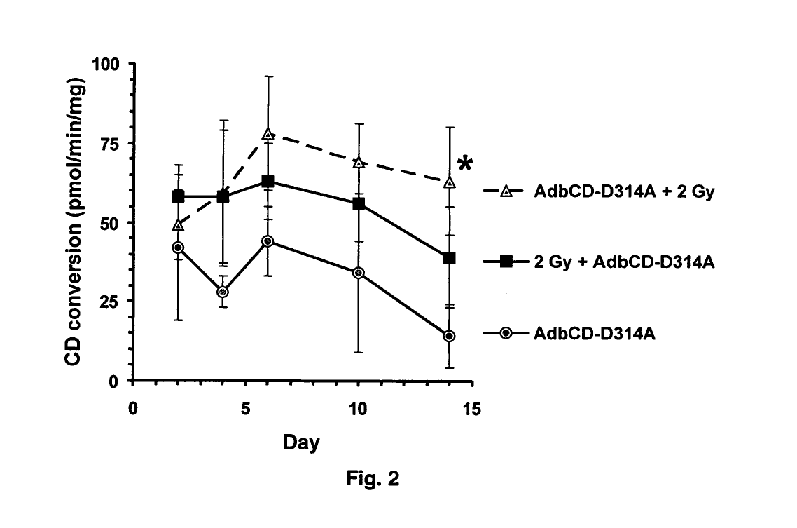

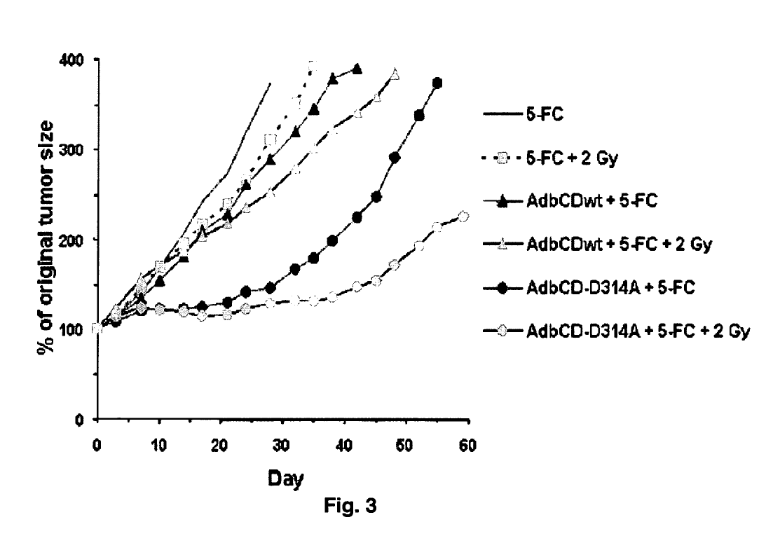

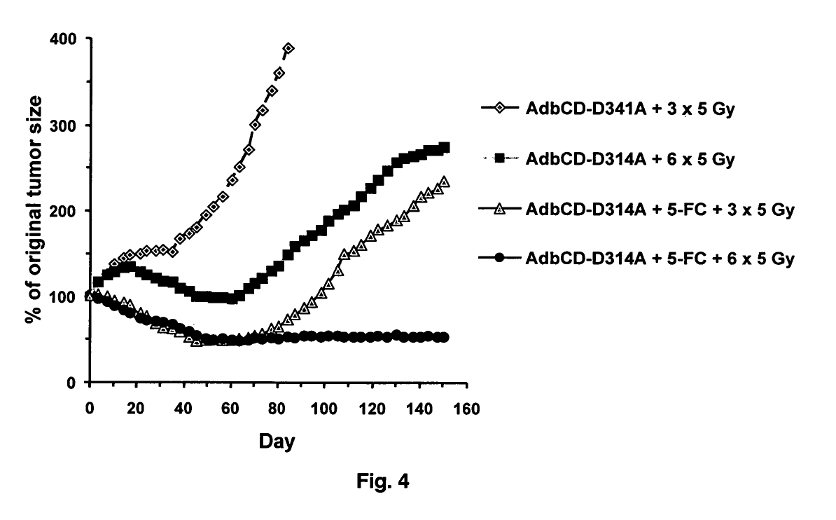

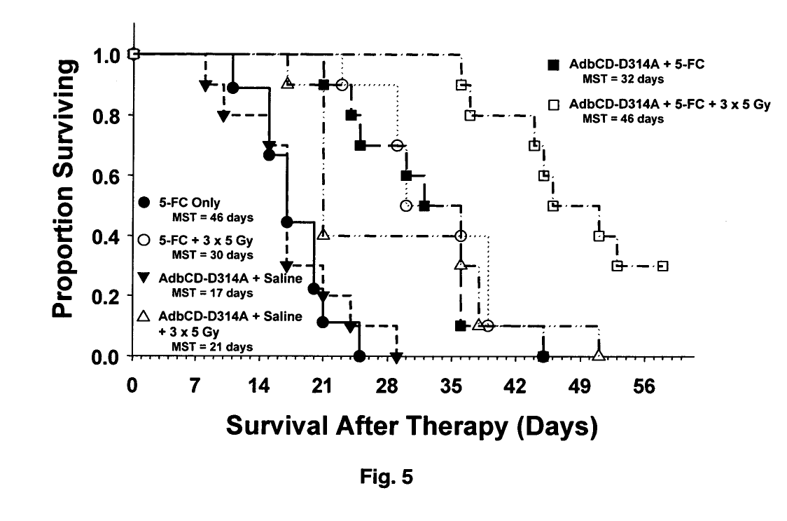

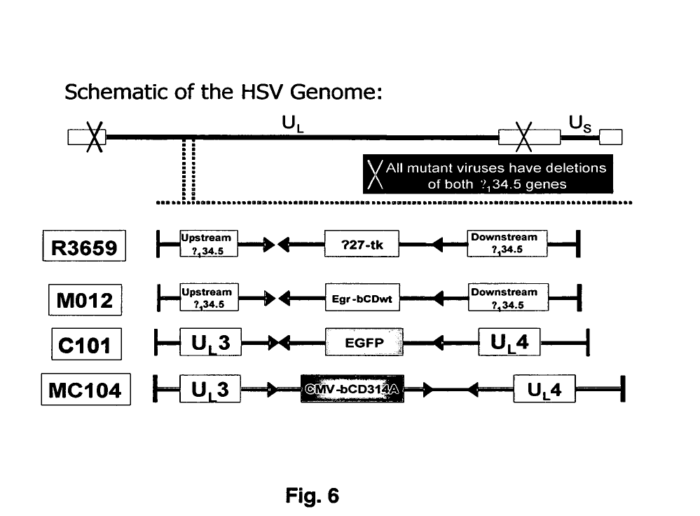

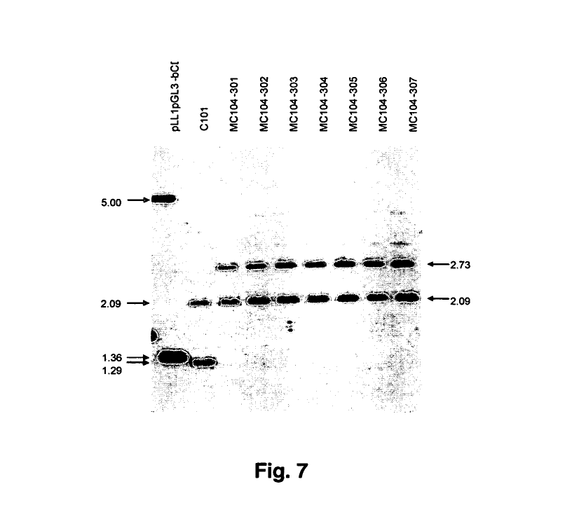

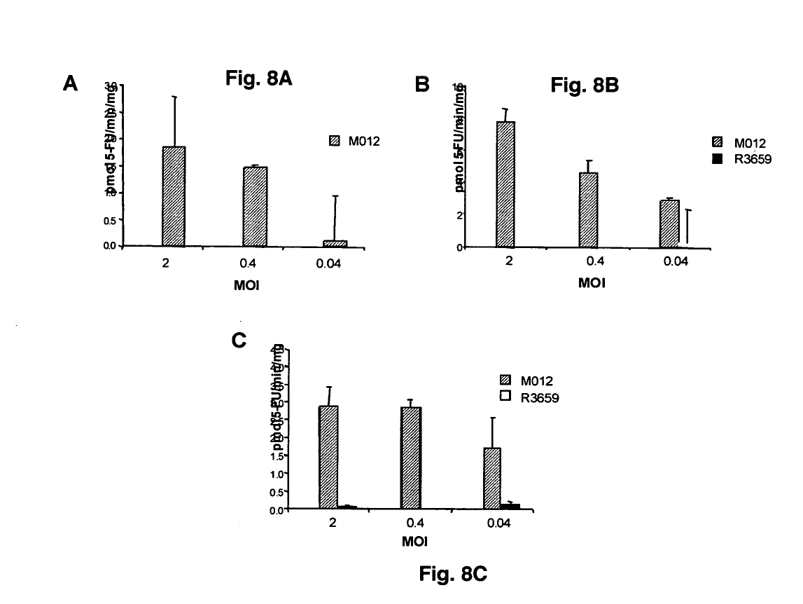

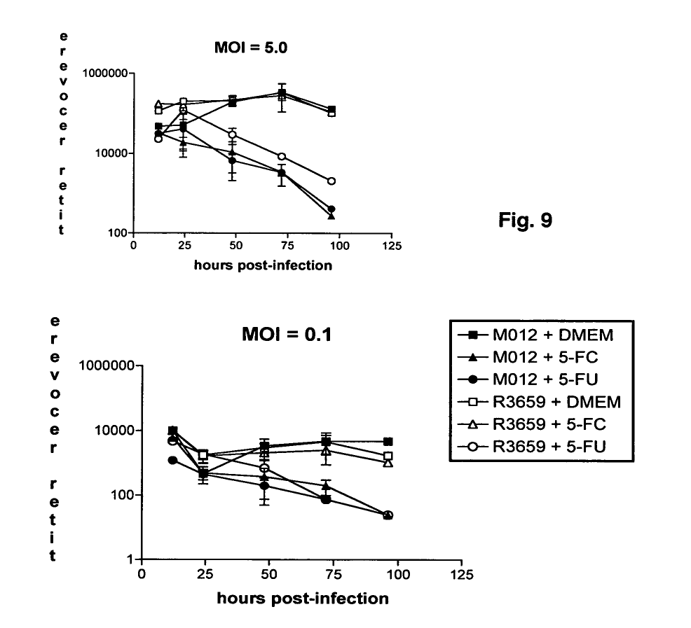

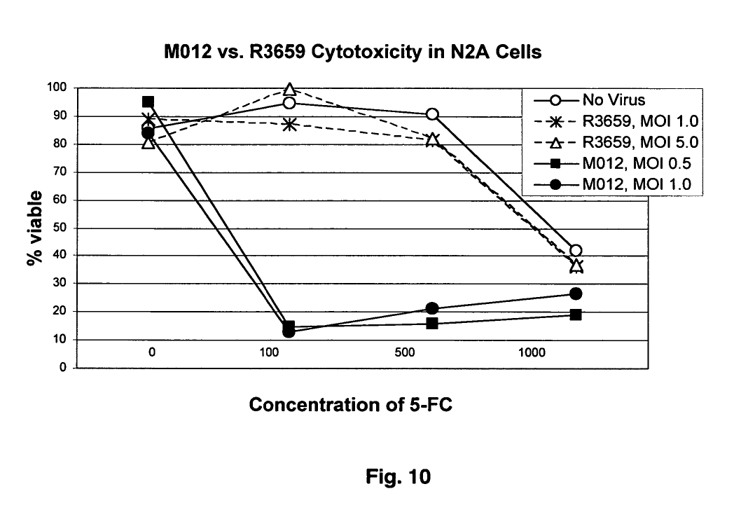

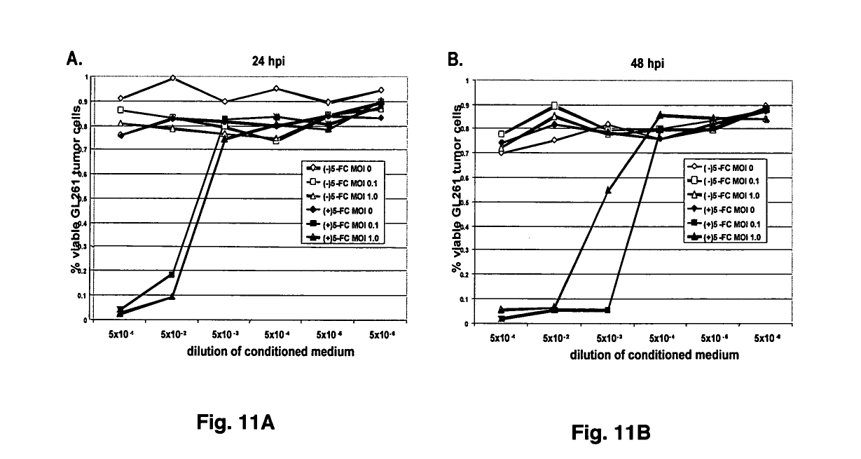

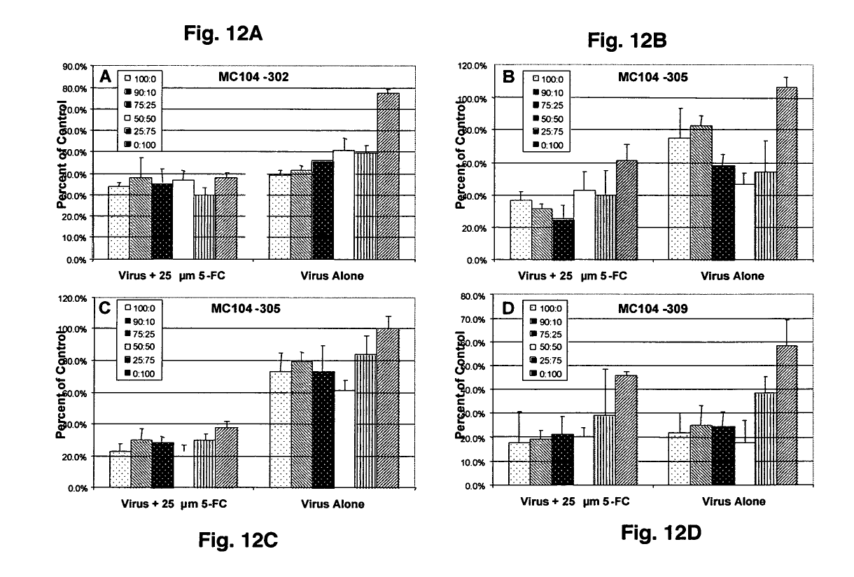

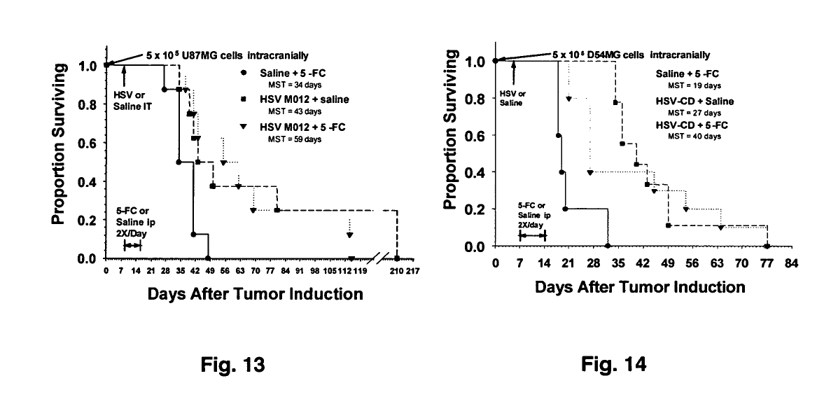

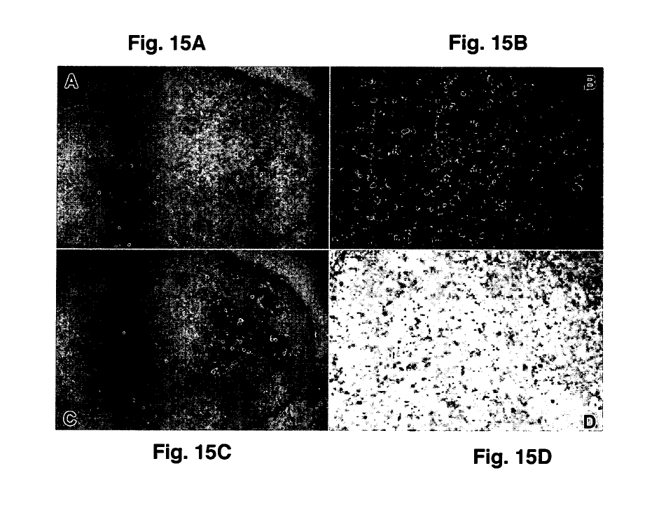

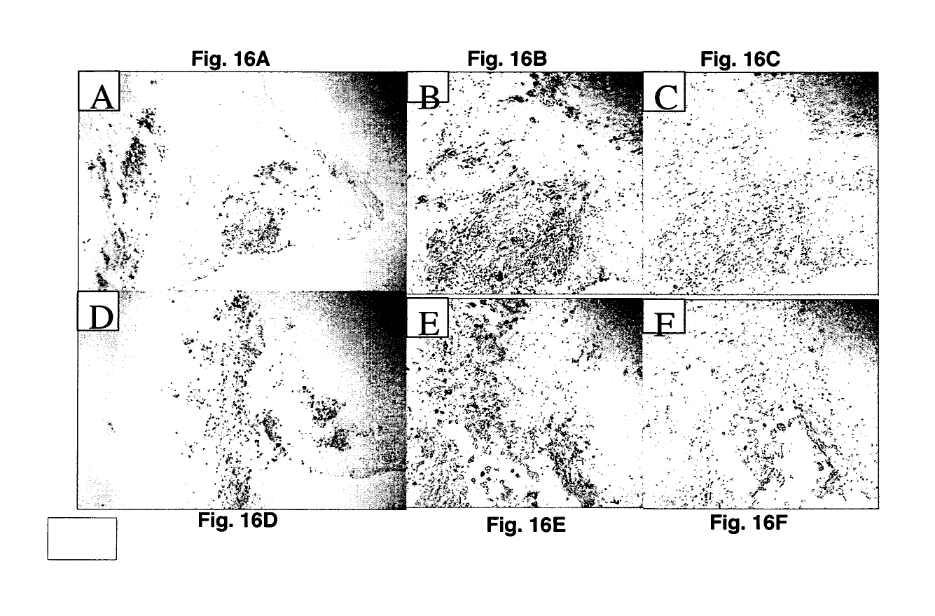

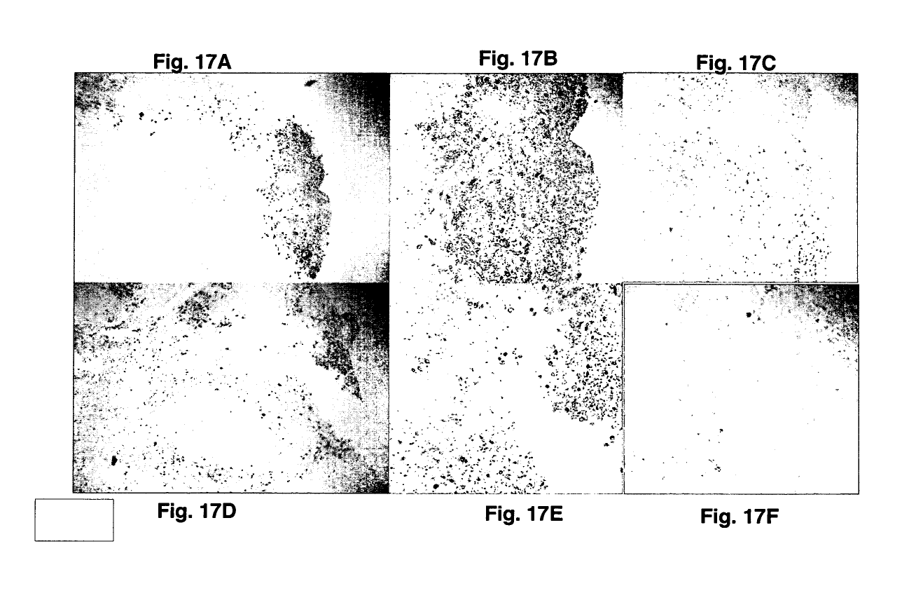

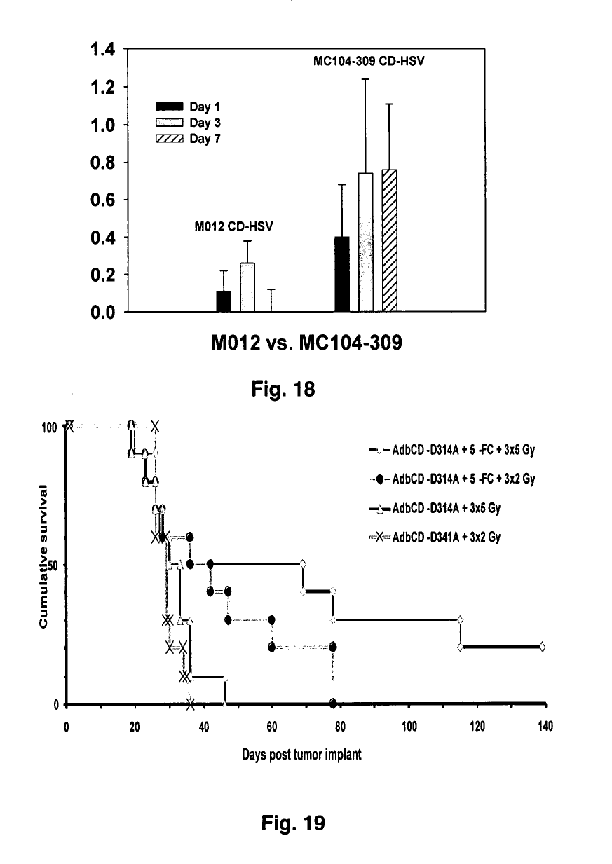

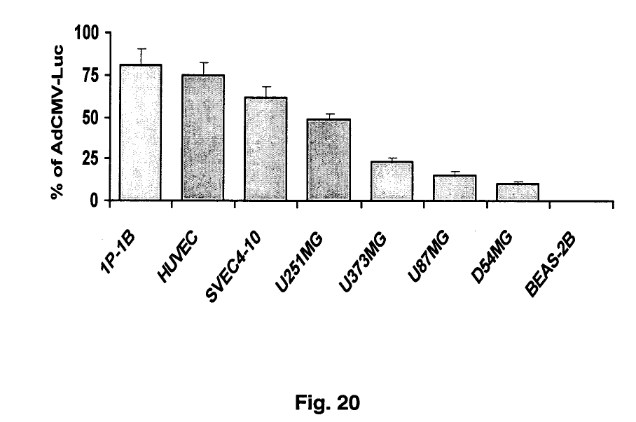

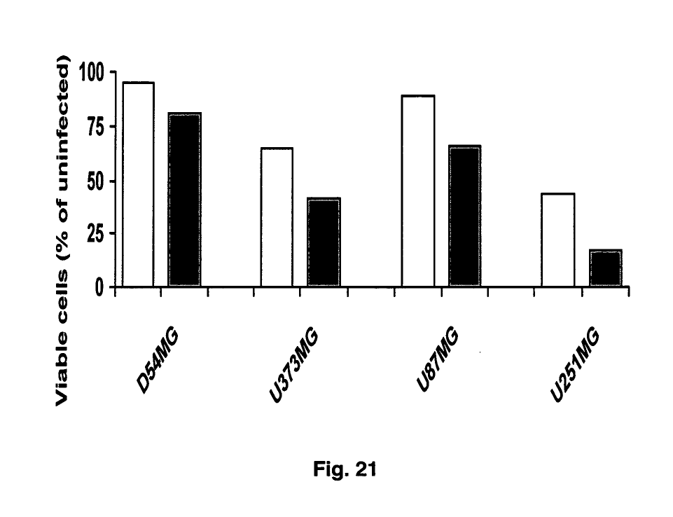

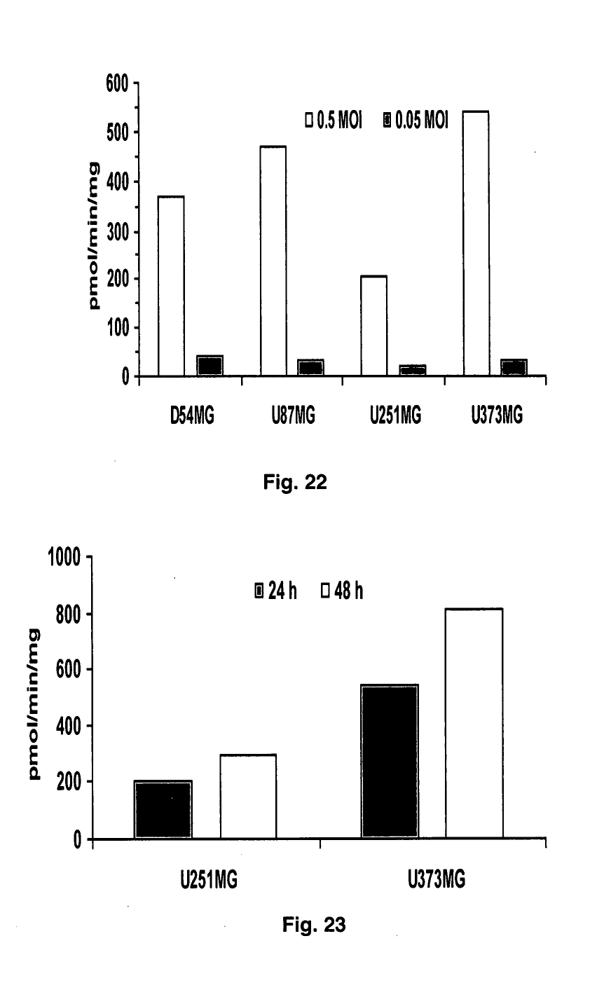

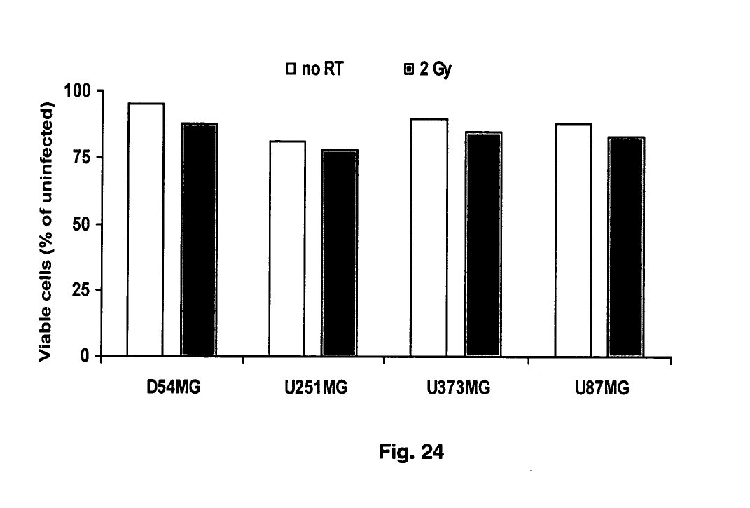

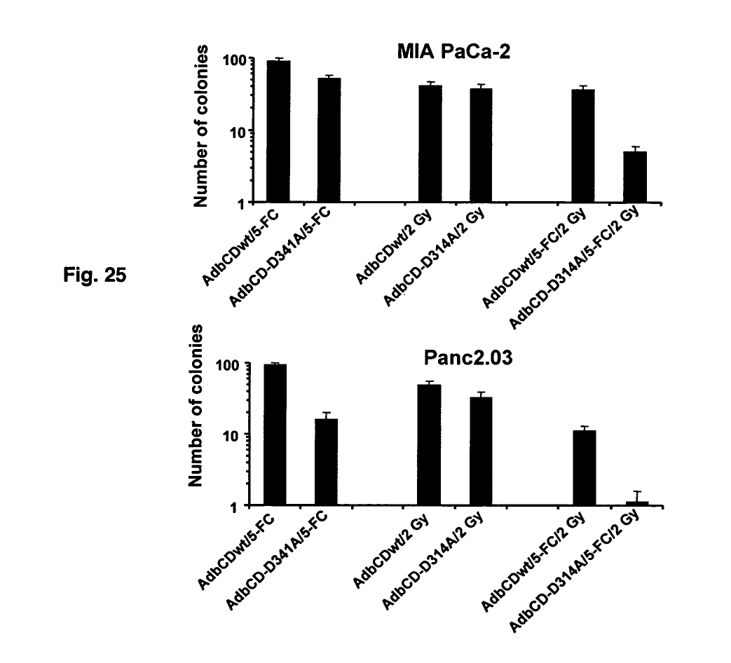

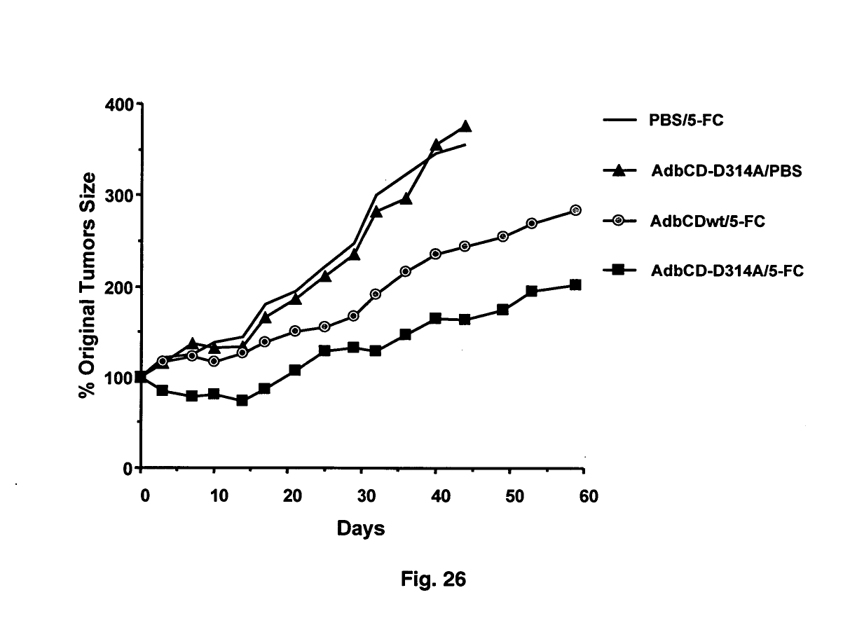

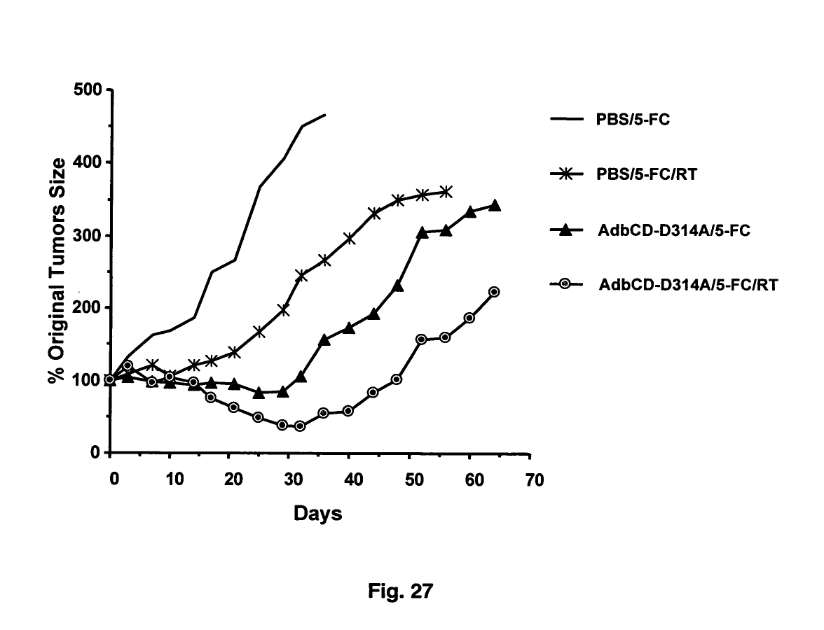

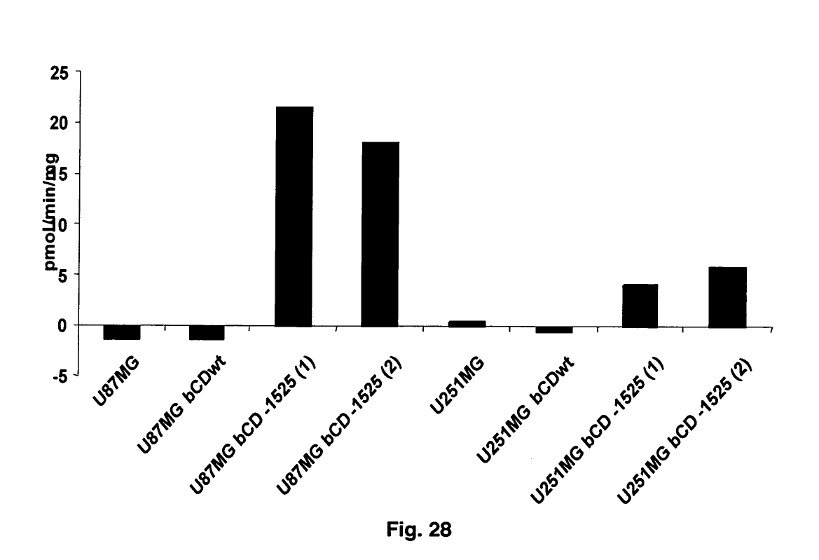

To test whether gene therapy using AdbCD-D314A/5-FC was able to modulate long-term cell survival in combination with radiation treatment, D54MG glioma cells were infected with 50 MOI AdbCD-D314A or AdbCDwt, exposed to ionizing radiation at 2 Gy and survival determined using a clonogenic survival assay. AdbCDwt infected D54MG cells demonstrated a reduction in the number of cell colonies to 86% after 2 Gy, 62% after incubation with 5-FC at 4 μg/ml and 92% after combination AdbCDwt/5-FC with radiation treatment in comparison with untreated control cells (FIG. 1). There was a significantly greater reduction in the number of AdbCD-D314A infected D54MG cell colonies to 7.5% after incubation with 5-FC and 0.28% after combined treatment with AdbCD-CD314A/5-FC and radiation in comparison with AdbCD-D314A infected cells. Also, treatment with AdbCD-D314A/5-FC enhanced the radiation induced U87MG and U251MG glioma cell death, and the cytotoxic effect improved as the MOI of AdbCD-D314A was increased (data not shown).

Enzyme Activity of bCDwt and bCD-D314A in D54MG Glioma Xenografts

Taking into consideration the results of previous experiments, D54MG glioma cells were selected for subsequent animal studies of combined suicide gene therapy with radiation treatment because they were the most resistant for in vitro treatment with 5-FU and bCDwt suicide gene therapy, and thus provided the most stringent test of the efficacy of this therapy. The CD enzyme activity was determined by measuring the conversion of ³H-5-FC to ³H-5-FU in D54MG glioma xenografts after intratumoral (i.t.) injection with AdbCD-D314A or AdbCDwt (FIG. 2). The results of CD conversion assays demonstrated that enzyme activity was elevated 303-fold at 2 days after Ad injection in tumors injected with AdbCD-D314A (40.9 μmol/min/mg) in comparison with AdbCDwt injected tumors (0.135 μmol/min/mg). Also, combination AdbCD-D314A infection with radiation treatment increased CD enzyme activity in D54MG tumors (FIG. 2). At 14 days after infection, there was a significant increase in enzyme activity in D54MG glioma xenografts treated with radiation one day after AdbCD-D314A infection (63.0±18.5 pmol/min/mg) in comparison with AdbCD-D314A alone (14.1±11.4 μmol/min/mg, p=0.018). In contrast, there were not significant differences in CD conversion values between D54MG tumors injected with AdbCD-D314A one day after irradiation (38.8±26.7_pmol/min/mg) versus AdbCD-D314A alone (p=0.267) and AdbCD-D314A one day before versus after irradiation (p=0.215).

In Vivo Therapy of D54MG Glioma Xenografts Using Suicide Gene Therapy Alone or in Combination with Radiation Treatment

To further evaluate the therapeutic potential of combination suicide gene therapy with radiation treatment in vivo, D54MG cells were subcutaneously (s.c.) injected into the flank of athymic nude mice. Before treatment, the mean tumor sizes in groups of 10 mice at baseline were not significantly different between treatment groups (p>0.05), and the within treatment variances were not significantly different (p>0.05). The baseline mean and standard deviation for tumor sizes at 14 days post tumor cell injection was 70.1±18.6 mm². In vivo tumor therapy was initiated on Day 0, which corresponded to 14 days post-tumor cell injection. Animals were injected i.t. with 1×10⁸ TCID₅₀ AdbCD-D314A or AdbCDwt on Days 0, 7 and 14. 5-FC was administered i.p. at 500 mg/kg twice daily 5 days/week for 3 weeks. Three groups of mice received radiation treatment at 2 Gy on Days 4, 7 and 10. An inhibition of D54MG tumor growth was noted in groups of mice treated with AdbCD-D314A/5-FC alone and AdbCD-D314A/5-FC in combination with radiation treatment versus the 5-FC-injected group (FIG. 3). There were no significant differences in tumor growth between groups that received 5-FC plus radiation treatment versus 5-FC alone, 5-FC plus radiation versus AdbCDwt/5-FC, or AdbCDwt/5-FC versus AdbCDwt/5-FC in combination with radiation treatment (p>0.05). The mean time to tumor doubling for 5-FC alone, 5-FC plus radiation, AdbCDwt/5-FC, AdbCDwt/5-FC plus radiation, AdbCD-D314A/5-FC and AdbCD-D314A/5-FC in combination with radiation treatment groups were 13, 15, 16, 17, 38 and 54 days, respectively. Comparisons of mean time to tumor doubling of the group treated with AdbCD-D314A/5-FC in combination with radiation versus AdbCD-D314A/5-FC alone and AdbCD-D314A/5-FC versus AdbCDwt/5-FC plus radiation showed significant differences between the groups (p<0.05).

To test whether increased doses of radiation combined with suicide gene therapy of s.c. D54MG glioma xenografts had greater efficacy, athymic nude mice were irradiated with three or six 5 Gy fractions after AdbCD-D314A/5-FC treatment (FIG. 4). Before treatment, the mean tumor sizes in groups of 10 mice at baseline were not significantly different between treatment groups (p>0.05), and the within treatment variances were not significantly different (p>0.05). In vivo tumor therapy was initiated on Day 0, which corresponded to 17 days post-tumor cell injection. Animals were injected it. with 1×10⁸ TCID₅₀ AdbCD-D314A on Days 0, 7 and 14. 5-FC was administered i.p. at 500 mg/kg twice daily 5 days/week for 3 weeks. Tumors received radiation treatment at 5 Gy on Days 4, 7 and 10 or 4, 7, 10, 14, 17 and 21. The mean time to tumor doubling for AdbCD-D314A plus 3×5 Gy, AdbCD-D314A plus 6×5 Gy, AdbCD-D314A/5-FC plus 3×5 Gy groups was 51, 109 and 137 days in comparison AdbCD-D314A/5-FC plus 6×5 Gy which produced about a 50% reduction tumor size at day 150, the last day of the study.

Intracranial Glioma Therapy Model

The potential clinical efficacy of suicide gene therapy using mutant bCD-D314A was assessed in an intracranial athymic nude mouse model of D54MG human glioma. Data from a preliminary study showed that AdbCD-D314A/5-FC treatment prolonged survival of mice bearing intracranial D54MG tumors in comparison with mock-infected animals treated with 5-FC or AdbCD-D314A alone (24 days versus 13 days). To test whether the combination of suicide gene therapy with radiation is more effective than either treatment alone, mice bearing intracranial D54MG tumors were irradiated with 5 Gy fractions on Days 1, 3 and 7 after a single i.t. injection of AdbCD-D314A on Day 0 (6 days post-tumor cell injection), plus a 2 week course of i.p. 5-FC at 500 mg/kg twice daily on Days 0 to 4 and 7 to 11. As shown in FIG. 5, the median survival times for 5-FC, AdbCD-D314A, AdbCD-D314A plus radiation, 5-FC plus radiation, AdbCD-D314A/5-FC alone, and AdbCD-D314A/5-FC in combination with radiation therapy were 17, 17, 21, 30, 32 and 46 days, respectively. AdbCD-D314A/5-FC in combination with radiation therapy significantly prolonged survival of mice in comparison with 5-FC alone, AdbCD-D314A alone, AdbCD-D314A plus radiation, and AdbCD-D314A/5-FC alone (p<0.05). Also, AdbCD-D314A plus 5-FC prolonged survival of mice in comparison with 5-FC alone and AdbCD-D314A alone.

Construction of Recombinant HSV Vectors Containing the Genes for bCDwt or bCD-D314A

The schematics of the recombinant HSV vectors described below are shown in FIG. 6. The coding sequence for E. coli bCDwt was introduced into both g₁34.5 loci under transcriptional control of the Egr-1 promoter. The tk gene was restored within M012 and candidate tk-repaired clones were plaque purified in Vero cells, then screened by Southern blot hybridization. The presence of the repaired tk gene in M012, as well as the presence of bCDwt, was also confirmed by Southern analysis. The candidate clone with highest CD conversion activity was chosen for all subsequent studies and designated M012.

Since construction of M012, a more efficient technique for construction of most viruses was adapted. MC104 was constructed under this paradigm using parent virus C101. C101 is a Dg₁34.5 HSV that contains an insertion of EGFP surrounded by PacI sites in the U_{L}3-U_{L}4 intergenic locus. C101 viral DNA was digested with PacI and co-transfected with a shuttle plasmid containing bCD-D314A and HSV flanking sequences. Candidate clones were plaque purified in Vero cells, then screened by Southern blot hybridization. The presence of bCD-D314A was also confirmed by Southern analysis (FIG. 7). The candidate clone with highest CD conversion activity was chosen for all subsequent studies and was designated MC104. A comparison of the in vitro efficiency of 5-FC conversion to 5-FU produced by infection with the initial HSV bCDwt expressing construct, M012 was undertaken (Table 19). The results are shown for U87MG (FIG. 8A), D54MG (FIG. 8B), and U251MG (FIG. 8C). This virus made significant amounts of active bCDwt, with increasing conversion seen at higher MOI.

TABLE 19
Conversion activity of wild-type and mutant bCD HSV
vectors in cells infected with 0.4 PFU of each virus

| | Conversion (pmol/mg/min) | | | |
|---|---|---|---|---|
| | D54MG | U87MG | U251MG | Vero |
| M012 | 10.9 | 18.8 | 15.4 | 2.4 |
| MC104-301 | 48.1 | 94.8 | 80.1 | 150.6 |
| MC104-302 | 57.1 | 126.8 | 41.8 | 152.6 |
| MC104-303 | 54.4 | 129.2 | 45.5 | 121.0 |
| MC104-304 | 14.2 | 51.9 | 0.0 | 14.4 |
| MC104-305 | 58.0 | 79.0 | — | 112.6 |
| MC104-309 | 70.3 | 143.4 | 68.7 | 106.6 |
| MC104-311 | 60.5 | 225.3 | 63.9 | 95.0 |

To determine if the addition of ionizing radiation would increase the conversion of 5-FC to 5-FU by our HSV bCDwt construct, human glioma cells were infected with virus in monolayer culture. Additionally, cells were treated with either 3.3 Gy radiation 24 h prior to infection with HSV or 24 h after infection with HSV. A third group of cells received no radiation. Treatment with radiation tended to increase the efficiency of 5-FC conversion, with the greatest increases seen in D54MG, U87MG, and U251MG cells treated with radiation 24 h prior to HSV infection (data not shown).

Replication of M012 In Vitro

To establish replication competence of M012 in murine tumor cell lines in vitro, Neuro-2a murine neuroblastoma cells were infected with either the parent Dg₁34.5 HSV, R3659, or M012 using a low (0.1) or high (5.0) MOI. To determine if high concentrations of 5-FC might interfere with HSV replication, cells were incubated in the presence of either 500 μM 5-FC or 50 μM 5-FU, or media alone and infected cells were harvested at 12, 24, 48, 72, and 96 h post-infection. Total PFU recovered at each time point was determined by plaque titration on Vero cells (FIG. 9). At both MOI, M012 replication in DMEM alone was similar to its parent virus, R3659. The addition of 5-FU to the growth medium resulted in a 1.0-2.0 log reduction in replication of either virus first evident at 48 h post-infection, and the inhibition was more pronounced at 72 and 96 h. The addition of 5-FC to the growth medium following infection inhibited M012 replication, but had no effect on R3659 replication at either MOI. Inhibition of M012 replication by 5-FC was most significant at the high MOI, as compared to R3659 replication in the presence of 5-FC (P=0.002).

Compared to the Neuro2a neuroblastoma cells studied above, human glioma cells are much more permissive for HSV infection. To determine if 5-FC administration would impair HSV replication, and decrease the benefit of the 5-FU anti-tumor effect in human glioma cells, the replication of M012 and parent virus R3659 (no CD insert) was examined in the presence of 0, 100, 500, and 1,000 micromolar 5-FC. U87MG, U251MG, and D54MG glioma cell lines were tested. No appreciable changes in viral replication was seen at any concentration of 5-FC tested. This lack of effect was present in all three human glioma cell lines, suggesting that an additional benefit of the HSV-CD VDEPT model might be found in malignant glioma in vivo models when compared to neuroblastoma.

Cytotoxicity Produced by M012 Infection and Effects of 5-FC Administration

Cytotoxic effects of M012 and R3659 infection were compared in Neuro-2a cells in the presence of increasing concentrations of 5-FC. The addition of 5-FC did not appreciably increase cytotoxicity in Neuro-2a cells infected with R3659 at 5-FC concentrations less than 1 mM. However, 5-FC concentrations of 1 mM or greater were toxic to Neuro-2a cells as evident by mock-infected controls. Under the conditions of this experiment, direct viral effects on Neuro-2a cells were limited. Even a high MOI (5.0) of R3659 had minimal cytotoxic effect on Neuro-2a cells, and M012/5-FC effects were well demonstrated. R3659 and M012 in the absence of 5-FC showed no difference in cytotoxicity. However, 5-FC conferred increased toxicity to Neuro-2a cells infected by M012 in a prodrug dose-dependent manner (FIG. 10). With 5-FC concentrations as low as 100 μM, a significantly greater cytotoxic effect was seen in M012-infected cells infected at lower MOI when compared to R3659 infected at a higher MOI (P<0.001). At 500 μM 5-FC, at all MOIs tested, M012 was more effective at killing Neuro-2a cells than R3659 (P<0.001). At 1,000 μM 5-FC, tumor cells were less viable than with lower concentrations of 5-FC, but the additional M012 effect over R3659 remained demonstrable (for R3659 MOI=5.0, P<0.001, and for R3659 MOI=1.0, P<0.032). There appeared to be a greater M012/5-FC effect at higher MOI, perhaps owing to increased bCDwt expression. Under conditions where direct viral lytic effects are limited (as demonstrated by minimal cytolytic effect at 0 μM 5-FC independent of MOI), prodrug-mediated cytotoxicity is more apparent at higher MOI of M012. M012 also showed an effect at an MOI as low as 0.1, but this effect was not seen until the concentration of 5-FC was 500 μM or greater.

Whether M012-infected cells would produce a diffusible cytotoxin in the presence of 5-FC capable of killing tumor cells was determined next. Vero cells were infected with M012 in the presence or absence of 1 mM 5-FC. At 24 or 48 h post-infection, ‘conditioned media’ were collected from M012-infected cells, which, when grown in the presence of 5-FC, should contain both 5-FU and progeny virions. To demonstrate 5-FU cytotoxicity independent of cytotoxicity owing to direct cell lysis by virus, M012-conditioned media was tested on the HSV-1-resistant GL261 murine glioma cells. This cell line is resistant to infection by both wild-type and Dg₁34.5 HSV-1, even at high MOI (1000) [37]. GL261 tumor cells were inoculated with the M012-conditioned media at dilutions ranging from 5×10⁻¹ to 5×10⁻⁶ of the original concentration, and grown for 7 days. Cell viability was assayed by alamarBlue. Supernates were collected from M012 or mock-infected cells cultured with or without 5-FC. Media from M012-infected Vero cells grown in the presence of 5-FC killed GL261 tumor cells, whereas the media collected from infected cells grown in the absence of 5-FC did not. At both 24 and 48 h post-infection (FIG. 11), a CD/5-FC effect was observed. For GL261 cells exposed to the 5-FC/M012 ‘conditioned’ medium, there was a greater cytotoxic effect observed at the later time point (48 h). Additionally, M012/5-FC ‘conditioned’ medium produced a more dramatic effect on tumor cell proliferation at 48 h when obtained from cells cultured at lower viral MOI: a 17-fold decrease in viability at MOI=0.1 vs. only a 1,5-fold decrease at MOI=1.0. Without 5-FC, supernates from cells cultured with M012 at 0.1 and 1.0 MOI exhibited only minimal effect on GL261 viability when compared to those from mock-infected cells. As a control, addition of 5-FC alone to GL261 cells did not produce significant cytotoxicity. These studies demonstrated that a non-viral cytotoxic agent was present in the media of cells infected with M012 and treated with 5-FC. This is consistent with the known bystander properties of diffusible 5-FU produced by CD in M012-infected tumor cells treated with 5-FC on adjacent, uninfected cells.

Cytotoxicity Produced by MC104 Infection and Effects of 5-FC Administration.

A similar assay, described in Example 19, was employed to determine the ability of three different clones of the bCD-D314A expressing HSV, MC104, to infect human glioma cells and convert 5-FC to kill co-cultured mouse glioma cells (GL261). GL261 is relatively insensitive to Dg₁34.5 HSV while D54MG, U87MG and U251MG human glioma cell lines are relatively sensitive. By coculturing these two cell types together in different ratios, one can assess the ability of the mutant CD, generated when MC104 HSV infects human glioma cells, to convert 5-FC to kill the HSV-insensitive GL261 cells. Human glioma cells D54MG (FIG. 12A), U87MG (FIGS. 12B, 12D) or U251MG (FIG. 12C) were mixed 100:0, 90:10, 75:25, 50:50, 25:75 or 0:100 with mouse GL261 glioma cells and plated at 4,000 total cells per well in 96 well plates. After overnight incubation, the cells were infected with 1 MOI (4,000 PFU) of 1 of 3 clones of MC104 HSV for 4 h or not and either medium alone or containing 25 mM 5-FC was added to the wells. After 72 h, the numbers of surviving cells were estimated by addition of alamar Blue and reading the color change at 590 and 562 nm in a microplate reader. These clones exhibited varying levels of direct oncolysis (Virus Alone) and bystander killing by 5-FU converted by expressed bCD-D314A (Virus+25 mM 5-FC).

CD Activity After Infection of Glioma Xenografts with M012

To assay CD activity in vivo, Neuro-2a flank tumors in A/J mice were treated with 5×10⁷ PFU of M012 or R3659, homogenized at different times and 1% of each tumor's volume analyzed for CD activity by TLC. Results demonstrated recovery of functional CD protein at each time point with significant activity detectable above control to day 8 (Table 20). CD produced by M012 was able to convert high concentrations (250 μM) of 5-FC with a mean conversion of 33% of tritiated 5-FC to 5-FU at 48 h post-infection (range 27-50%). Analysis of CD activity between the two treatment groups and compared over time showed statistically significant differences between the two groups (P<0.0001).

TABLE 20
CD activity demonstrated in M012-infected Neuro-2a flank tumors.

| Virus treatment | CD activity^{a,b} | | | |
|---|---|---|---|---|
| group | Day 2^{c} | Day 4 | Day 6 | Day 8 |
| M012 | 33% (27-50) | 5.00% (0.6-11.6) | 1.20% (0.6-1.9) | 0.67% (0.6-0.8) |
| R3659 | 0.35% (0.3-0.4) | 0.17% (0.33-0.41) | 0.34% (0.23-0.52) | 0.23% (0.18-0.25) |

^{a}As mean percent (range) recovered from 1% of HSV-infected flank tumor homogenates.
^{b}P < 0.0001 for the two treatment groups.
^{c}Day post-virus treatment of tumor.

To evaluate M012 viral replication in Neuro-2a tumors in A/J mice, flank tumors were injected with 5×10⁷ PFU of either M012 or R3659, followed by i.p. injections of either saline or 5-FC. Virus titers of tumor homogenates (PFU/g clarified tissue) revealed no statistical difference (P>0.1) in viral replication kinetics between M012 and R3659 independent of 5-FC administration (data not shown), unlike the findings in vitro.

Antitumor Activity of M012 Vector with 5-FC

Studies were undertaken to determine the efficacy of the HSV construct, M012, in in vivo murine glioma models. In a study using U87MG cells that were implanted in the right frontal lobe of SCID mice, M012 given with 5-FC produced the longest median survival (59 days), while mice treated with M012 and saline had a median survival of only 43 days (FIG. 13). The median survival of animals receiving saline plus 5-FC was 34 days. In a similar study with the D54MG intracranial model, median survival of mice injected intratumorally with M012 and administered 5-FC was 40 days, compared to 27 days with M012 plus saline and 19 days with saline and 5-FC (FIG. 14).

M012 neurovirulence of the CD-expressing virus (PFU/LD₅₀) following intracerebral inoculation was assessed as follows: HSV-susceptible A/J mice were injected intracerebrally with escalating doses (6.6×10⁶, 2×10⁷ and 6×10⁷ PFU) of M012 (three mice/group) in 10 μl total volume. Beginning 1 day before virus injection, 1 ml of 5-FC (10 mg/ml) was administered twice daily for a total of 7 days. No deaths occurred in any of the groups after monitoring the mice for 30 days. Furthermore, none of the animals appeared ill, even at the highest dose tested (limited by the titer of the virus stock). Thus, the maximum tolerated dose (PFU/LD₅₀) of M012 combined with 5-FC administration was >6×10⁷ PFU in A/J strain mice. Similar findings were seen in the absence of 5-FC administration.

Immunohistochemistry of M012 in Tumors

Studies were undertaken to evaluate the spread of the M012 virus within U87MG subcutaneous xenografts. As shown in FIG. 15, there was widespread tumor necrosis with an advancing edge of M012 at 4 days after intratumor injection. There was a direct correlation between HSV and bCDwt staining.

MC104-309 HSV (Supplemental Data U54)

The present invention characterized “second generation” genetically engineered HSV (MC104-309) containing the mutant version of bacterial cytosine deaminase (CD) designated D314A-CD. In two series of studies, the present invention compared this second generation CD-HSV with the first generation CD-HSV (M012;) [32] in ability to replicate, spread and persist in D54MG human gliomas established in athymic nude mice. The data suggests that the MC104-309 HSV has equal or better replication properties in subcutaneous glioma xenografts compared to the first generation virus, has equal or better capacity to spread throughout these gliomas and the mutant CD converts between 3-6 fold more 5-FC prodrug to 5-FU. In FIG. 16, the distribution of either the M012 (Panels FIG. 16A-16C) or the MC104-309 HSV (Panels 16D-16F) are roughly equivalent at 3 days in these representative immunohistochemistry sections stained for HSV antigens (FIGS. 16A, 16B, 16D, 16E). Expression of either the wild-type CD (FIG. 16C) or the mutant CD (FIG. 16F) closely mirrors the distribution of the viruses. At 7 days (FIG. 17), the tumors injected with M012 CD-HSV (FIG. 17A-17C) had a persistent but more localized expression pattern than those infected with MC104-309 CD-HSV (FIG. 17D-17F). Both viruses continued to demonstrate immunohistochemically detectable expression of Cytosine Deaminase, but the apparent level of expression appeared to be reduced from that seen at Day 3. All tumors were processed and stained at the same time to minimize batch variability in the staining procedures. The present invention also titered virus recovered from each tumor at Days 3 and 7. No attempt was made to titer virus at 24 hours since it would have represented a significant amount of input virus. A comparable amount of infectious virus was found at both time points for both viruses. However, when the gliomas were homogenized and independently assayed for the ability of the expressed CD enzyme to convert tritiated 5-FC to tritiated 5-FU, there was a striking difference between the two viruses (FIG. 18). The conversion activity in the D54MG gliomas injected with the M012 CD-HSV was 3-6 fold lower at all time points (Days 1, 3 and 7) than that seen in the D54MG gliomas that were injected with MC104-309. Moreover, at Day 7 when infectious virus was still detectable, the activity of the wild-type CD was undetectable in these tumors. In contrast, the conversion activity was still present in and at a comparable level to Day 3 in the gliomas injected with the mutant CD-HSV, MC104-309. These data suggest that the second generation Cytosine Deaminase HSV expressing the mutant version of CD is likely to generate a much greater anti-tumor effect than we have observed with the M012 HSV.

Ad Vectors can Encode the D314A Mutant of CD and Work Synergistically with Ionizing Radiation

The potential clinical efficacy of GDEPT was examined using the D314A mutant of bacterial cytosine deaminase (bCD) encoded by Ad vectors in intracranial athymic nude mouse models of glioma. To test whether the combination of molecular chemotherapy with radiation is more effective than either treatment alone, mice bearing intracranial D54MG tumors were irradiated in multiple fractions (3×5 Gy) after single i.t. injection of AdbCD-D314A followed by a twice daily, 2 week course of i.p. 5-FC at 500 mg/kg. As shown in FIG. 19, this treatment protocol significantly prolonged survival of mice (SF50=52 days) in comparison with mock-infected animals treated with 5-FC, AdbCD-D314A alone, AdbCD-D314A plus radiation or AdbCD-D314A/5-FC (23, 23, 27 or 37 days, respectively; p<0.05).

Flt-1 Promoter Activity in Glioma Cell Lines

For initial screening of flt-1 promoter activity, several human glioma cell lines were infected with Adflt-Luc or AdCMV-Luc recombinant Ad. Forty-eight h after infection cells were harvested and luciferase expression was analyzed by luciferase assay system (FIG. 20). The results demonstrate highest flt-1 promoter activity in blood vessel endothelial cells and U251 MG and U373MG glioma cells.

Transcriptional and Transductional Targeted CRAds for Glioma Oncolysis

A CRAd (Conditionaly Replicative Adenovirus) was developed using the flt-1 promoter element for specific E1a gene expression (CRAdflt-1). A major issue of Ad gene therapy is the fact that most tumor cells as well as endothelial cells demonstrate low levels of CAR expression. To address such limitations that may occur in primary gliomas, a retargeted CRAd was developed employing the flt-1 promoter to control E1a gene expression with a RGD-4C peptide inserted into the HI-loop of the Ad fiber knob domain (CRAdRGDflt-1). To evaluate the oncolytic activity of CRAdflt-1 and CRAdRGDflt-1, several glioma cell lines were infected at 1 MOI (FIG. 21). The human glioma cells demonstrated different levels of cytolysis after CRAdflt-1 infection. There was a viral dose-dependent correlation in cell killing, measured by crystal violet staining assay. Enhanced cell death following CRAdRGDflt-1 infection in comparison with CRAdflt-1 (wild-type Ad fiber knob) occurred in all the glioma cell lines, and to the greatest extent in the U251MG and U373MG cell lines.

Development and Testing of CRAd Vector Expressing Mutant bCD

We have developed a replication-competent CRAdRGDflt-bCD-D314A vector with E1b and E3 gene deletion using the flt-1 promoter element for E1a gene expression and the CMV promoter for D341A mutant bCD gene expression. Four human glioma cell lines (D54MG, U87MG, U251MG, and U373MG) were infected with 0.5 or 0.05 MOI and the next day the cells were harvested and tested for ³H-5-FC to ³H-5-FU conversion. The conversion activity utilizing 1 μg lysates (0.5 MOI) or 10 μg lysates (0.05 MOI) is summarized in FIG. 22. The CRAdRGDflt-bCD-D314A resulted in conversion of the pro-drug 5-FC to the active drug 5-FU in infected cells at a rate equal to 200-550 μmol/mg/min (0.5 MOI) and 20-45 μmol/mg/min (0.05 MOI). There are approximately 3-fold greater levels of 5-FU production than occurred with the non-replicative AdbCD-D341A vector at 2 MOI. Also, CD conversion was elevated an additional 1,4-fold and 1,5-fold in U251MG and U373MG cells, respectively, on Day 2 after infection with CRAdRGDflt-bCD-D314A (FIG. 23).

To evaluate the oncolytic activity of CRAdRGDflt-bCD-D314A vector, we infected D54MG, U251MG, U373MG and U87MG glioma cell lines at 0.1 MOI (FIG. 24). The human glioma cells demonstrated different levels of cytolysis at 6 days after CRAdRGDflt-bCD-D314A infection. There was a viral dose-dependent correlation in cell killing, measured by crystal violet staining assay (data not shown). The highest level of cell death following CRAdRGDflt-bCD-D314A infection occurred in the U251MG glioma cell line. The comparisons of number of viable glioma cells for groups that were treated with the CRAdRGDflt-bCD-D314A/5-FC alone or in combination with radiation treatment showed no significant differences between the groups.

To determine the 5-FC sensitivity of tumor cells to CRAdRGDflt-bCD-D314A-mediated suicide gene expression, human glioma cells were infected with 0.1 MOI of CRAdRGDflt-bCD-D314A, treated with increasing concentrations of 5-FC, and the relative cell viability was determined using the crystal violet staining assay. To investigate whether combined treatment with CRAdRGDflt-bCD-D314A/5-FC and radiation treatment produced increased cytotoxicity of cancer cells in vitro, human glioma cells were irradiated with 2 Gy the day after infection. As shown in Table 21, the combination of CRAdRGDflt-bCD-D314A infection and 2 Gy radiation treatment one day later produced significantly enhanced D54MG and U373MG cell death in comparison with CRAdRGDflt-bCD-D314A and 5-FC treatment (p<0.05). The IC₅₀ values for D54MG shown in Table 21 are lower by 4.7 and 4,0-fold than for these cells infected with 10 MOI of non-replicative AdbCD-D314A/5-FC alone and in combination with radiation treatment one day after infection, respectively.

TABLE 21
Cytotoxicity of combination treatment with radiation
and molecular chemotherapy against human glioma cells

| | 5-FC IC₅₀ (μg/ml)^{a} | | | |
|---|---|---|---|---|
| | D54MG | U251MG | U373MG | U87MG |
| CRAdRGDflt-bCD-D314A | 11.3 ± 2.3 | 39.7 ± 4.2 | 8.4 ± 1.2 | 5.1 ± 0.8 |
| CRAdRGDflt-bCD-D314A + 2 Gy | 6.9 ± 0.8 | ND | 4.6 ± 0.6 | ND |
| ^{a}Cells were infected with 0.1 MOI of CRAdRGDflt-bCD-D314A, and mock-irradiated or irradiated with 2 Gy using a ⁶⁰Co gamma irradiator one day after Ad infection. Relative cell density was determined using a crystal violet staining assay. Data shown in comparison with control CRAdRGDflt-bCD-D314A infected cells without 5-FC added. The combination of CRAdRGDflt-bCD-D314A/5-FC treatment and irradiation one day later produced significantly enhanced cell death in comparison with | | | | |
| # mock-irradiated CRAdRGDflt-bCD-D314A/5-FC cells (p < 0.05). | | | | |

These results demonstrate that the recently developed conditionally replicating Ad encoding mutant bCD produces a much higher level of 5-FU and cytotoxicity, and has the potential to significantly enhance human glioma therapy by this molecular chemotherapy approach in combination with radiotherapy.

CD Conversion in Tumor Xenografts

For CD conversion assay, 7×10⁶ Panc2.03 pancreatic cancer cells were injected s.c. into female athymic nude mice. When tumors reached 6-8 mm in diameter, they were injected with 1×10⁷ TCID₅₀ of AdbCD-D314A. Also, 1.6×10⁷ MIA PaCa-2 cells were resuspended 1:1 (v/v) in Matrigel (Becton Dickinson, San Jose, Calif.) and s.c. injected into female athymic nude mice. MIA PaCa-2 tumors were injected with 5×10⁷ TCID₅₀ of AdbCDwt or 5×10⁷ TCID₅₀ of AdbCD-D314A. Tumors were dissected at different times after Ad injection, and one third of the tumor was crushed with a mortar and pestle in 100-200 μl Complete Mini protease inhibitor buffer (Roche Diagnostics, Indianapolis, Ind.). Tumor tissues were frozen and thawed three times, and after centrifugation the supernatant assayed for protein concentration and subjected to CD conversion assay as described above.

Subcutaneous Human Pancreatic Tumor Xenograft Studies

To compare anti-tumor effects of AdbCD-D314A and AdbCDwt mediated molecular chemotherapy on established solid tumors, 7×10⁶ Panc2.03 pancreatic cancer cells were injected s.c. into female athymic nude mice. Treatment was started 11 days post-tumor cell injection at the time of established tumor growth (tumors were 6-8 mm in diameter), noted as Day 0. Animals were randomly divided into groups receiving different treatments: 1) AdbCDwt plus 5-FC; 2) AdbCD-D314A plus 5-FC; 3) AdbCD-D314A plus PBS; 4) PBS plus 5-FC. Mice were injected i.t. with 1×10⁸ TCID₅₀ per tumor AdbCDwt or with 1×10⁷ TCID₅₀ AdbCD-D314A on Days 0, 7, and 14. Three groups of mice (# 1, 2 and 4) received 5-FC i.p. at 400 mg/kg on Days 1 to 5, 8 to 12, and 15 to 19.

To evaluate in vivo therapeutic efficacy of the combination of AdbCD-D314A/5-FC and radiation treatment, 16×10⁶ MIA PaCa-2 tumor cells were resuspended 1:1 (v/v) in Matrigel (Becton Dickinson) and injected s.c. into female athymic nude mice. Treatment was started 17 days post-tumor cell injection at the time of established tumor growth, noted as Day 0. Animals were randomly divided into groups receiving different treatments: 1) AdbCD-D314A plus 5-FC; 2) AdbCD-D314A plus 5-FC in combination with radiation; 3) PBS plus 5-FC in combination with radiation; 4) PBS plus 5-FC. Two groups of mice (# 1 and 2) were injected i.t. with 5×10⁷ TCID₅₀ AdbCD-D314A on Days 0, 7, and 14. Radiation treatment for two groups of animals (#2 and 3) was 2 Gy from a ⁶⁰Co gamma irradiator (Picker Unit) on days 2, 9 and 16. All mice were treated i.p. with 400 mg/kg of 5-FC on Days 1 to 5, 8 to 12, and 15 to 19. Tumor size was monitored twice a week using digital Vernier calipers. Tumor surface areas were calculated as width×length (mm²).

Enzyme Activity of bCDwt and bCD-D314A In Vitro

The present invention constructed the replication-defective recombinant Ad vectors AdbCDwt and AdbCD-D314A, encoding wild type codA and mutant codA (harboring D314A mutation) genes, respectively, under control of the CMV promoter. In order to determine the CD enzyme activity, human pancreatic cancer cells were infected with 25 MOI AdbCD-D314A or AdbCDwt and the CD enzyme activity was determined by measuring the conversion of ³H-5-FC to ³H-5-FU. The results of CD conversion assays using thin layer chromatography (Table 22) demonstrated that the CD conversion activity was significantly (p-value<0.001) elevated 336, 406, 105, 160, 917 and 507-fold for MIA PaCa-2, BxPC-3, PANC-1, Panc2.03, S2-VP10 and S2-013 cells, respectively, following infection with AdbCD-D314A, in comparison with the level of conversion activity in AdbCDwt infected cells.

TABLE 22
The in vitro CD conversion activity of AdbCD-D314A
and AdbCDwt in pancreatic cancer cells^{a}

| | MIA PaCa- | BxPC-3 | PANC-1 | Panc2.03 | S2-VP1 [symbol] | S2-013 |
|---|---|---|---|---|---|---|
| AdbCDwt | 0.4 ± 0.3 | 0.6 ± 0.1 | 2.5 ± 0.4 | 0.7 ± 0.0 [symbol] | 0.2 ± 0.0 [symbol] | 0.2 ± 0.04 |
| AdbCD-D314A | 134.6 ± 6. [symbol] | 243.9 = 1 [symbol] | 262.9 ± 63 [symbol] | 112.0 ± 1. [symbol] | 183.4 ± 6 [symbol] | 101.4 ± 6.4 |

^{a}Cells were infected with 25 MOI of AdbCD-D314A or AdbCDwt and CD conversion activity was determined as pmol/min/mg by measuring the conversion of ³H-5-FC to ³H-5-FU.

5-FU Sensitivity of Pancreatic Cancer Cells In Vitro

To determine the sensitivity of cancer cells to 5-FU, pancreatic cancer cells were treated with increasing concentrations of 5-FU, and the cytotoxicity of this drug was determined by measuring surviving cells using the crystal violet staining method. The susceptibility to cytotoxic effects of 5-FU was variable in different pancreatic cancer cell lines (Table 23). Cell killing was proportional to the concentration of 5-FU used, and the range of concentration of 5-FU to produce 50% viable cells (IC₅₀, 50% inhibitory concentration) was from 0.1 μg/ml for BxPC-3 cells to 12.4 μg/ml for the Panc2.03 cell line (Table 23). The relative sensitivity to 5-FU treatment was BxPC-3>S2-013>MIAPaCa-2≧PANC-1>S2-VP10>Panc2.03.

TABLE 23
The cytotoxicity of 5-FU in human pancreatic
cancer cells in vitroa

| MIA Pa [symbol] | BxPC-3 | PANC-1 | Panc2.03 | S2-VP10 | S2-013 |
|---|---|---|---|---|---|
| 1.4 ± [symbol] | 0.1 ± 0.2 | 1.5 ± 0.5 | 12.4 ± 2.6 | 2.7 ± 0.1 | 0.5 ± 0.1 |

^{a}Cells were incubated with serial dilutions of 5-FU, and IC₅₀ (μg/ml) was determined at 5 days using crystal violet staining assay.

AdbCDwt/5-FC Sensitivity Alone and in Combination with Ionizing Radiation

To determine the sensitivity of tumor cells to molecular chemotherapy, human pancreatic cancer cells were infected with 50 MOI AdbCDwt or AdbCD-D314A, treated with increasing concentrations of 5-FC, and the relative cell viability was determined using the crystal violet staining assay. Expression of the mutant bCD-D341A protein in pancreatic cancer cells significantly increased their sensitivity to 5-FC treatment (Table 24). The IC₅₀ with 5-FC administration decreased by 4,9-fold for MIA PaCa-2, 12.3-fold for BxPC-3, 38.5-fold for PANC-1, 35.0-fold for Panc2.03, 7.2-fold for S2-VP10 and 6,3-fold for S2-013 in AdbCD-D341A infected cells in comparison with AdbCDwt infected cells (p<0.02). The viability of AdbCD-D314A infected cells incubated with 5-FC decreased in a MOI-dependent manner (data not shown). Uninfected cells treated with 5-FC at 764 μg/ml had no detectable cytotoxicity. As shown in Table 24, the combination of AdbCD-D314A/5-FC treatment and ionizing radiation one day later produced enhanced cell death in comparison with AdbCD-D314A/5-FC alone.

Additionally, these results were confirmed using a long-term clonogenic survival assay. The day after infection with 50 MOI AdbCD-D341A or AdbCDwt, Panc2.03 and MIA PaCa-2 pancreatic cancer cells were treated with 5-FC at 4 μg/ml, and the next day were exposed to radiation at 2 Gy and were subjected to clonogenic survival assay (FIG. 25). Radiation treatment alone caused a dose-dependent reduction in cell survival of uninfected cells (results not shown). AdbCD-D341A plus 5-FC treatment of Panc2.03 and MIA PaCa-2 cells produced a reduction in the number of colonies by 83 and 49%, respectively, compared to AdbCDwt/5-FC treated cells. Also, radiation treatment of AdbCD-D341A infected Panc2.03 and MIA PaCa-2 cells at 2 Gy reduced the number of colonies by 68 and 63%, respectively, in comparison with untreated cells (FIG. 25). There was a significantly greater reduction in the number of AdbCD-D341A/5-FC plus radiation treated Panc2.03 and MIA PaCa-2 cell colonies (94%, p=0.002 and 91%, p=0.0003, respectively) in comparison with AdbCD-D341A/5-FC alone treated cells. A similar reduction in the number of colonies following treatment with AdbCD-D314A/5-FC plus radiation was obtained for BxPC-3, PANC-1, S2-VP10 and S2-013 cells. Combined treatment using radiation and AdbCD-D341A/5-FC enhanced cell killing, and the cytotoxic effect improved as the MOI of AdbCD-D341A or concentration of 5-FC were increased.

Conversion Activity of bCDwt and bCD-D314A in Human Pancreatic Tumor Xenografts

Taking into consideration the results of in vitro experiments, Panc2.03 and MIA PaCa-2 pancreatic cancer cell lines with different sensitivities to 5-FU were selected for an animal model study. The CD enzyme activity was determined by measuring the conversion of ³H-5-FC to ³H-5-FU in MIA PaCa-2 tumor xenografts after single intratumoral (i.t.) injection of AdbCD-D314A or AdbCDwt recombinant vectors. The conversion activities on Day 2 and 7 post-injection of AdbCD-D314A in MIA PaCa-2 xenografts (3 tumors per group) were 5.7±2.3 and 4.5±2.1 μmol/min/mg, respectively, in comparison with undetectable (less than 0.01 pmol/min/mg) levels after AdbCDwt injection. A similar increase in CD conversion activity was obtained with AdbCD-D314A in Panc2.03 xenografts (n=3): 0.4±0.3, 2.4±0.4 and 2.7±0.7 pmol/min/mg, on Days 1, 4 and 11 after AdbCD-D314A injection, respectively, compared to undetectable levels in AdbCDwt injected tumors.

In Vivo Therapy of Human Pancreatic Cancer Xenografts Using Molecular Chemotherapy Alone or in Combination with Radiation Treatment

A preliminary dose-escalation study showed no differences in the mean time to tumor doubling for PBS plus 5-FC and AdbCDwt injected at 1×10⁷ TCID₅₀/mouse plus 5-FC. Thus, for subsequent experiments, injection with 1×10⁸ TCID₅₀/mouse of AdbCDwt and 10×10⁷ TCID₅₀/mouse of AdbCD-D314A were used. To further evaluate the therapeutic potential of AdbCD-D314A mediated GDEPT in vivo, 7×10⁶ Panc2.03 pancreatic cancer cells were s.c. injected into the flank of athymic nude mice. Before treatment, the mean tumor sizes in groups of 9-10 mice at baseline were not significantly different between treatment groups (p>0.05), and the within treatment variances were also not significantly different (p>0.05). In vivo tumor therapy was initiated on Day 0, which corresponded to 11 days post-tumor cell injection. The baseline mean and standard deviation for tumor sizes on Day 0 was 34.2±3.6 mm². The mean time to tumor doubling for PBS plus 5-FC, AdbCD-D314A alone, AdbCDwt plus 5-FC, and AdbCD-D314A plus 5-FC groups were 22, 23, 34, and 59 days, respectively (FIG. 26). There were no significant differences in tumor sizes between groups that received 1×10⁸ TCID₅₀/mouse of AdbCDwt plus 5-FC in comparison with PBS plus 5-FC treatment alone or AdbCD-D314A plus PBS (p>0.05). Comparisons of mean tumor volumes of the AdbCD-D314A at 1×10⁷ TCID₅₀/mouse plus 5-FC treatment group versus 1×10⁸ TCID₅₀/mouse of AdbCDwt plus 5-FC group showed significant differences between the groups (p=0.02).

Additionally we determined whether combination of molecular chemotherapy with radiation therapy increases tumor growth inhibition. For this study, MIA PaCa-2 cells were s.c. injected into the flank of athymic nude mice. Although the combination of molecular chemotherapy and radiation treatment produced only moderately increased cytotoxicity in vitro, the results of animal experiments demonstrated a significant delay in tumor growth following AdbCD-D314A plus 5-FC treatment in combination with radiation therapy in comparison with AdbCD-D314A plus 5-FC (FIG. 27). Before treatment, the mean tumor sizes in groups of 10 mice at baseline were not significantly different between treatment groups (p>0.05), and the within treatment variances were not significantly different (p>0.05). In vivo tumor therapy was initiated on Day 0, which corresponded to 17 days post tumor cell injection. The baseline mean and standard deviation for tumor sizes on Day 0 was 52.2±8.5 mm². The mean time to tumor doubling for PBS plus 5-FC, PBS plus 5-FC in combination with radiation, AdbCD-D314A plus 5-FC and AdbCD-D314A plus 5-FC in combination with radiation treated groups were 14, 30, 45, and 62 days, respectively. Comparisons of mean tumor volumes showed significant differences between the AdbCD-D314A plus 5-FC in combination with radiation treatment group compared with PBS plus 5-FC in combination with radiation as well as AdbCD-D314A plus 5-FC treated mice (p=0.003 and p=0.01, respectively). Moreover, in the group treated with AdbCD-D314A plus 5-FC in combination with radiation, 4/10 of the tumors underwent complete regression, in contrast to no regressions in PBS plus 5-FC, PBS plus 5-FC in combination with radiation and AdbCD-D314A plus 5-FC treated groups.

TABLE 24
The effects of combination radiation treatment and
molecular chemotherapy of human pancreatic cancer cells

| | 5-FC IC₅₀ (μg/ml)^{a} | | | |
|---|---|---|---|---|
| | | | | AdbCD- |
| | AdbCDwt | AdbCDwt + IR | AdbCD- | D314A |
| MIA | 104.2 ± 19.2 | 53.7 ± 10.1 | 21.5 ± 6.8 | 8.7 ± 5.0 |
| PaCa-2 | | | | |
| BxPC-3 | 3.7 ± 0.5 | 2.2 ± 0.3 | 0.3 ± 0.1 | 0.1 ± 0.1 |
| PANC-1 | 42.3 ± 3.5 | 24.1 ± 4.9 | 1.1 ± 1.2 | 0.8 ± 0.5 |
| Panc2.03 | 397.4 ± 92.4 | 172.1 ± 77.2 | 11.3 ± 5.3 | 4.7 ± 2.4 |
| S2-VP10 | 185.4 ± 74.5 | 82.5 ± 21.5 | 25.7 ± 12.1 | 14.3 ± 3.5 |
| S2-013 | 58.7 ± 24.4 | 29.3 ± 10.5 | 9.3 ± 0.9 | 5.7 ± 0.7 |

^{a}Cells were infected with 50 MOI of AdbCD-D314A or AdbCDwt, on the next day fresh media supplemented with serial dilutions of 5-FC were added, and 24 h after prodrug treatment cells were mock-irradiated or irradiated with 2 Gy using a C [symbol] mma irradiator (+IR). 5-FC cytotoxicity was determined on day 5 after Ad infection b [symbol] crystal [symbol] let staining assay and cell survival was corrected for background absorbance [symbol] radi [symbol] n treatment alone.

The instant invention constructed the plasmid encoding 1525 mutant bCD under control of the CMV promoter (pShuttleCMV-1525) that will be used to produce novel Ad vectors. We evaluated the 5-FC to 5-FU conversion activity of human glioma cell lines after transfection with plasmids encoding two clones of the 1525 mutant or bCDwt gene. Results of conversion with 1525 mutant plasmid are summarized in FIG. 28 and illustrate [symbol] much greater conversion activity than bCDwt. Likewise, we expect greater conversion activity when the non-replicative and replicative Ad vectors expressing bCD-1524 are produced. These results demonstrate that the mutant bCD Ads have the potential to significantly enhance glioblastoma therapy by this molecular chemotherapy approach in combination with radiotherapy.

The following references were cited herein.
1 Dachs G U et al., Anticancer Drugs 2005; 16: 349-359.
2 Yazawa K et al., World J Surg 2002; 26: 783-789.
3 Mullen C A et al., Proc Natl Acad Sci USA 1992; 89: 33-37.
4 Lawrence T S et al., Int J Radiat Oncol Biol Phys 1994; 29: 519-523.
5 Ichikawa T et al., Cancer Gene Ther 2000; 7: 74-82.
6 Kambara H et al., Cancer Gene Ther 2002; 9: 840-845.
7 Miller C R et al., Cancer Res 2002; 62: 773-780.
8 Kurozumi K et al., J Neurooncol 2004; 66: 117-127.
9 Conrad C et al., Cancer Gene Ther 2005; 12: 284-294.
10 Diasio R B et al., Antimicrob Agents Chemother 1978; 14: 903-908.
11 Mahan S D et al., Protein Eng Des Sel 2004; 17: 625-633.
12 Mahan S D et al., Biochemistry 2004; 43: 8957-8964.
13 Brandes A A et al., Semin Oncol 2003; 30: 4-9.
14 Stackhouse M A et al., Gene Ther 2000; 7: 1019-1026.
15 Patterson A V et al., Curr Pharm Des 2003; 9: 2131-2154.
16 Pederson L C et al., Cancer Res 1997; 57: 4325-4332.
17 Kim J H et al., Cancer J Sci Am 1998; 4: 364-369.
18 Kievit E et al., Cancer Res (Suppl) 2000; 60: 6649-6655.
19 Adachi Y et al., Hum Gene Ther 2000; 11: 77-89.
20 Kaliberov S A, et al. Cancer Gene Ther 2006; 13: 203-214.
21 Moriuchi S et al. Gene Ther 2002; 9: 584-591.
22 Rogulski K R et al., Hum Gene Ther 1997; 8: 73-85.
23 Wang Z H et al., J Neuropathol Exp Neurol 1999; 58: 847-858.
24 Kanai F et al., Cancer Res 1998; 58: 1946-1951.
Kievit E et al., Cancer Res 1999; 59: 1417-1421.
26 Zhang M et al., Mol Ther 2003; 8: 21-28.
27 Qian J et al., Cancer Res 2005; 65: 5493-5497.
28 Kramm C M et al., Methods Mol Biol 2003; 215: 137-152.
29 Rainov N G et al., Cancer J 2003; 9: 180-188.
30 Sonabend A M et al., Rev Med Virol 2006; 16: 99-115.
31 Bradley J D et al., Clin Cancer Res 1999; 5: 1517-152253.
32 Guffey M B et al Cancer Gene Ther, September 22:Epub ahead of print, 2006.
33. Andreansky S et al. Proc Natl Acad Sci USA, 93:11313-11318, 1996.
34. Andreansky S et al Cancer Res, 57:1502-1509, 1997.
35. Andreansky S et al. Gene Ther, 5:121-130, 1998.
36. Markert J M et al. Rev Med Virol, 10:17-30, 2000.
37. Parker J N et al. Proc Natl Acad Sci USA, 97:2208-2213, 2000.
38. Markert J M et al. Herpes, 8:17-22, 2001.
39. Markert J M et al. Neurosurgery, 32:597-603, 1993.
40. Mineta T et al. Gene Ther, 1 Suppl 1:S78, 1994.
41. Yazaki T et al. Cancer Res, 55:4752-4756, 1995.
42. Markert J M et al. Gene Ther, 7:867-874, 2000.
43. Rampling R et al. Gene Ther, 7.859-866, 200053.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A recombinant adenovirus vector comprising:
a gene encoding a mutant cytosine deaminase operatively linked to a functional promoter; wherein said vector when transfected in a host, expresses cytosine deaminase in a biologically active form.

2. The recombinant adenovirus vector of claim 1, wherein the vector further comprises an arginine-glycine-aspartic acid (RGD) peptide in the fiber knob of said adenovirus.

3. The recombinant adenovirus vector of claim 1, wherein said promoter is the CMV or hTERT promoter.

4. The recombinant adenovirus vector of claim 1, wherein said mutant cytosine deaminase gene is a E. coli gene.

5. The adenovirus vector of claim 1, wherein said mutant gene harbors a substitution of an alanine for the aspartic acid at position 314 of the wild type cytosine deaminase gene.

6. The adenovirus vector of claim 1, wherein said mutant gene harbors substitution of an Alanine for Valine at position 152, a cysteine for the phenylalanine at position 316, and glycine for the aspartic acid at position 317 of the wild type cytosine deaminase gene.

7. The recombinant adenovirus vector of claim 1, wherein said adenoviral vector is a replication-deficient, adenovirus.

8. The recombinant adenoviral vector of claim 1, wherein said adenoviral vector is a conditionally replicative adenovirus.

9. The recombinant adenoviral vector of claim 1, wherein said adenovirus is under control of a tumor specific promoter.

10. The recombinant adenoviral vector of claim 9, wherein said tumor specific promoter is the flt-1 promoter.

11. A mutant Herpes Simplex Virus 1 vector comprising:
a gene encoding cytosine deaminase; and
a gene encoding uracil phosphoribosyl transferase; operatively linked to a functional promoter; wherein said vector when transfected to a host, expresses both the cytosine deaminase and uracil phosphoribosyl transferase in a biologically active form.

12. The mutant Herpes Simplex Virus 1 vector of claim 11, wherein said genes are cistronically linked to produce a fusion protein.

13. The mutant Herpes Simplex Virus 1 vector of claim 11, wherein said mutant Herpes Simplex virus vector contains deletion in both copies of the viral _{□1}34.5 gene.

14. The mutant Herpes Simplex Virus 1 vector of claim 11, wherein the promoter is selected from the group consisting of the CMV, Egr-1, TERT, sFLT promoter or a promoter of a gene specifically expressed in malignant cells.

15. The mutant Herpes Simplex Virus 1 vector of claim 11, wherein said cytosine deaminase gene is a E. coli gene.

16. The mutant Herpes Simplex Virus 1 vector of claim 11, wherein said cytosine deaminase gene is mutated.

17. The mutant Herpes Simplex Virus 1 vector of claim 16, wherein said mutant cytosine deaminase gene harbors a substitution of an alanine for the aspartic acid at position 314 of the wild type cytosine deaminase gene.

18. The mutant Herpes Simplex Virus 1 vector of claim 16, wherein said mutant cytosine deaminase gene harbors a substitution of an alanine for valine at position 152, a cysteine for the phenylalanine at position 316, and glycine for the aspartic acid at position 317 of the wild type cytosine deaminase gene.

19. The mutant Herpes Simplex Virus 1 vector of claim 11, wherein said uracil phosphoribosyl transferase gene is an E. Coli gene.

20. A method of causing selective growth inhibition of a malignant tumor in a mammal comprising:
introducing the genetically engineered vector of the composition of either claim 1 or claim 11 in the mammal; wherein the product of said vector is expressed in the malignant tumor and
administering 5-fluorocytosine, in said mammal.

21. The method of claim 20, further comprising of treating said mammal with radiation therapy.

22. The method of claim 21, wherein said mammal is a human, non-human primate, cow, sheep, horse, goat, mouse, gerbil, hamster, rabbit, dog, or cat.

23. The method of claim 20, wherein said tumor is selected from a group of central nervous system tumors consisting of, glioma, gliosarcoma, oligodendroglioma, astrocytoma, ependymoma, primitive neuroectodermal tumor, malignant meningioma, schwannoma, malignant peripheral nerve sheath tumor or neurobalstoma.

24. The method of claim 20, wherein said tumor is selected from a group consisting of malignant cells of the kidney, liver, bile duct, pancreas, lung, peritoneum, prostate, breast, uterus, skin, lips, mouth, throat, esophagus, stomach, bowel, colon and rectum.

25. The method of claim 20, wherein said 5-fluorocytosine is administered in a dosage of about 12.5 to 37.5 mg/kg of body weight every six hours.

26. The method of claim 20, wherein said radiation is applied at a daily dose of from about 1.8 Gy to about 2.2 Gy over a 6 week period.

27. A method of enhancing radiosensitization in a mammal in need thereof comprising:
administering to the mammal a genetically engineered viral vector of the composition of claim 1 or claim 11;
administering 5-fluorocytosine to the mammal; and
treating the individual with radiation therapy.

28. The method of claim 27, wherein said mammal is a human, non-human primate, cow, sheep, horse, goat, mouse, gerbil, hamster, rabbit, dog, or cat.

29. The method of claim 27, wherein said mammal is suffering from a tumor from a group of central nervous system tumors consisting of, glioma, gliosarcoma, oligodendroglioma, astrocytoma, ependymoma, primitive neuroectodermal tumor, malignant meningioma, schwannoma, malignant peripheral nerve sheath tumor or neurobalstoma.

30. The method of claim 27, wherein said mammal has a malignancy of the kidney, liver, bile duct, pancreas, lung, peritoneum, prostate, breast, uterus, skin, lips, mouth, throat, esophagus, stomach, bowel, colon and rectum.

31. The method of claim 27, wherein said 5-fluorocytosine is administered in a dosage of about 12.5 to 37.5 mg/kg of body weight every six hours.

32. The method of claim 27, wherein said radiation is applied at a daily dose of from about 1.8 Gy to about 2.2 Gy over a 6 week period.
